(19) United States
(12) Patent Application Publication
(10) Pub. No.: US 2024/0050555 A1
(43) Pub. Date: Feb. 15, 2024

(54) IMMUNOGENIC COMPOSITION USEFUL FOR VACCINATION AGAINST ROTAVIRUS

(71) Applicant: BOEHRINGER INGELHEIM VETMEDICA GMBH, Ingelheim am Rhein (DE)

(72) Inventors: Abby Rae PATTERSON, Story City, IA (US); David Michael ANSTROM, Ames, IA (US); Gregory Brian HAIWICK, Ankeny, IA (US); Wesley Scott JOHNSON, Ames, IA (US); Dianna M. Murphy JORDAN, Ames, IA (US); Bryon NICHOLSON, Ames, IA (US); Eric Martin VAUGHN, Ames, IA (US); Justin WIDENER, Athens, GA (US)

(21) Appl. No.: 18/295,675

(22) Filed: Apr. 4, 2023

Related U.S. Application Data
(60) Provisional application No. 63/362,488, filed on Apr. 5, 2022.

(51) Int. Cl. A61K 39/15; A61P 31/14; C12N 7/00

(52) U.S. Cl. CPC A61K 39/15; A61P 31/14; C12N 7/00; A61K 2039/6056

(57) ABSTRACT

The present invention relates to immunogenic compositions comprising recombinantly constructed polypeptides useful for preparing vaccines, in particular for reducing one or more clinical signs caused by a rotavirus infection. More particular, the present invention is directed to an immunogenic composition containing (i) a fusion protein comprising in N- to C-terminal direction (A) an immunogenic fragment of a rotavirus VP8 protein and (B) an immunoglobulin Fc fragment such as, for example, an IgG Fc fragment, and (ii) an immunogenic substance, different from said fusion protein, wherein said immunogenic composition is usable in a method of reducing one or more clinical signs, mortality or fecal shedding caused by a rotavirus infection in swine.

### CROSS-REFERENCE TO RELATED APPLICATIONS

This application claims priority to U.S. patent application 63/362,488 filed on Apr. 5, 2022, which is incorporated by reference herein, in its entirety.

### SEQUENCE LISTING

The Sequence Listing associated with this application is provided in XML format in lieu of a paper copy, and is hereby incorporated by reference into the specification. The name of the XML file containing the Sequence Listing is “20230404-APPLICATION-Sequence Listing-23-0015-US-2.xml”. The XML file is 34.50 KB; it was created on 28 Feb. 2023; and it is being submitted electronically via EFS-Web, concurrent with the filing of the specification.

### INCORPORATION BY REFERENCE

All references cited herein, are incorporated by reference herein, in their entirety.

### BACKGROUND OF THE INVENTION

### Technical Field

The present invention relates to immunogenic compositions comprising recombinantly constructed polypeptides useful for reducing one or more clinical signs caused by a rotavirus infection. More particular, the present invention is directed to an immunogenic composition containing (i) a fusion protein comprising in N- to C-terminal direction (A) an immunogenic fragment of a rotavirus VP8 protein and (B) an immunoglobulin Fc fragment such as, for example, an IgG Fc fragment, and (ii) an immunogenic substance, different from said fusion protein, wherein said immunogenic composition is usable in a method of reducing one or more clinical signs, mortality or fecal shedding caused by a rotavirus infection in swine.

### Background Information

Rotaviruses are double-stranded RNA viruses which comprise a genus within the family Reoviridae. Rotavirus infection is known to cause gastrointestinal disease and is considered the most common cause of gastroenteritis in infants. Rotavirus is transmitted by the faecal-oral route and infects cells that line the small intestine. Infected cells produce an enterotoxin, which induces gastroenteritis, leading to severe diarrhea and sometimes death through dehydration.

Rotaviruses possess a genome composed of 11 segments of double-stranded RNA (dsRNA) and are currently classified into eight groups (A-H) based on antigenic properties and sequence-based classification of the inner viral capsid protein 6 (VP6), as defined by the International Commitee on Taxonomy of Viruses (ICTV) and summarized by Matthijnssens et al. (Arch Virol 157:1177-1182 (2012)), wherein this and the following publications referred to herein are incorporated by reference in their entirety.

The genome of rotavirus encodes six structural proteins (VP1-VP4, VP6 and VP7) and six non-structural proteins (NSP1-NSP6), wherein genome segments 1-10 each encode one rotavirus protein, and genome segment 11 encodes two proteins (NSP5 and NSP6).

In the context of rotavirus A, different strains may be classified as genotypes (defined by comparative sequence analysis and/or nucleic acid hybridization data), or serotypes (defined by serological assays), based on the structural proteins VP7 and VP4. VP7 and VP4 are components of the outermost protein layer (outer capsid), and both carry neutralizing epitopes. VP7 is a glycoprotein (thus designated “G”) that forms the outer layer or surface of the virion. VP7 determines the G-type of the strain and the designations for G serotypes and G genotypes are identical. VP4 is protease sensitive (thus designated “P”) and determines the P-type of the virus. In contrast to the G-types the numbers assigned for P serotypes and genotypes are different (Santos N. et Hoshino Y., 2005, Reviews in Medical Virology, 15, 29-56). Therefore, the P serotype is designated as P followed by assigned number, and the P genotype is designated by a P followed by assigned number in brackets (e.g., “P[7]” or “P[13]”). Strains that belong to the same genotype have higher than 89% amino acid sequence identity (Estes and Kapikian. Rotaviruses. In: Knipe, D. M.; Howley, P. M. Fields Virology, 5th ed.; Wolters Kluwer/Lippincott Williams & Wilkins Health: Philadelphia, PA, USA (2007); Gorziglia et al. Proc Natl Acad Sci USA. 87(18):7155-9 (1990)).

Rotaviruses are in particular also a major cause of gastroenteritis in swine with antibodies against group A and C rotaviruses present in nearly 100% of pigs (Vlasova et al. Viruses. 9(3): 48 (2017)). Currently, only modified live or killed vaccines are available against rotavirus A. The inability to culture rotavirus C in the laboratory has hampered development of a vaccine against this group, which then adds to the attractiveness of a recombinant vaccine.

Generation of a recombinant anti-rotavirus vaccine is hindered by the complexity of the rotavirus capsid, which is composed of four proteins arranged in three layers. The innermost layer is composed of 60 dimers of VP2 with T=1 symmetry. The VP2 layer is required for proper ordering of the intermediate layer which is formed by 260 trimers of VP6 with T=13 symmetry. The resulting symmetry mismatch between VP2 and VP6 produces five distinct VP6 trimer positions and three distinct pore types. In the absence of VP2, VP6 readily forms ordered high molecular weight microtubules and spheroids in a salt and pH-dependent manner which may represent byproducts of viral assembly. In the capsid the VP6 layer is covered by 260 Ca2+-dependent trimers of VP7 which act as a clamp holding the VP4 spike in place. VP7 is the glycosylated or G-type antigen, and contains neutralizing epitopes. The majority of neutralizing antibodies recognize only trimeric VP7 and are thought to act by preventing dissociation of the VP7 trimer which in turn blocks release of the spike. Rotavirus spikes are present as 60 trimers of VP4 which are inserted into the VP6 layer only at pore type II. VP4 contains neutralizing epitopes and is the P-type antigen, cleaved by trypsin into spike base VP5* and cellular interaction head VP8*, which remains associated with VP5* following cleavage. Trypsinization primes the spike for cellular entry, during which the spike undergoes profound structural rearrangement to expose active sites for receptor binding on host cells. Ignoring the complexities of the above assembly process, stoichiometric expression of rotavirus capsid proteins with environmental conditions to promote proper assembly are difficult to achieve.

In light of the difficulty in rotavirus capsid assembly there was interest in a subunit vaccine approach. VP7 and VP4 are the two proteins that contain neutralizing epitopes, however use of VP7 would have been complicated by its glycosylation and calcium-dependent trimerization. Use of VP4 is complicated by its trimerization, trypsinization, and range of potential conformational states. The VP8 protein, also named VP8 domain or VP8*, which is produced by trypsinization of VP4 contains neutralizing epitopes, is monomeric, has had its structure determined to high resolution (Dormitzer et al. EMBO J. 21(5): 885-897 (2002)), and is described as highly stable.

Furthermore, within the VP8 protein, it is the lectin-like domain (aa65-224) which is considered to interact with the host receptor and to be involved in the attachment of the virus to the host cell (Rodriguez et al., PloS Pathog. 10(5):e1004157 (2014)).

Approaches to develop rotavirus subunit vaccines for children have been described, wherein a truncated VP8 protein (amino acid residues 64 (or 65)-223 of VP8*) N-terminally linked to the tetanus toxoid universal CD4⁺ T cell epitope (aa830-844) P2 was produced in Escherichia coli (Wen et al. Vaccine. 32(35): 4420-7 (2014)), and was tested in infants and toddlers (Groome et al. Lancet Infect Dis. 17(8):843-853 (2017)). However, as this use of a monovalent subunit vaccine (based on truncated VP8 protein of rotavirus genotype P[8]) elicited poor response against heterotypic rotavirus strains, also a trivalent vaccine formulation (comprising three proteins for combining genotypes P[4], P[6], P[8] antigens) was recently tested (Groome et al. Lancet Infect Dis. S1473-3099(20)30001 (2020)).

In another approach, an N-terminal truncated VP8 protein, “VP8-1” (aa26-241), was N-terminally or C-terminally fused with the pentamerizing nontoxic B subunit of cholera toxin (CTB). Of the resulting pentameric fusion proteins (CTB-VP8-1, VP8-1-CTB) only CTB-VP8-1 (i.e. VP8-1 N-terminally fused to CTB) was considered as a viable candidate for further development, as compared to VP8-1-CTB, it showed higher binding activity to GM1 or to conformation sensitive neutralizing monoclonal antibodies specific to VP8*, and elicited higher titers of neutralizing antibodies and conferred higher protective efficacy, in a mouse model (Xue et al. Hum Vaccin Immunother. 12(11) 2959-2968 (2016)).

However, in light of the difficulty in rotavirus capsid assembly there is an interest in alternative subunit vaccine approaches, in particular since subunit vaccines are generally considered to be very safe. Also, a recombinant expression of effective rotavirus subunit antigens is strongly desired which allows for the simple production of vaccine antigens of such rotaviruses which are difficult to culture. Furthermore, as rotaviruses are a major cause of gastroenteritis in swine, there is in particular a great need to have subunit vaccines for swine including antigens enabling an efficacy comparable to, or being even more efficient than, the MLV rotavirus vaccines currently commercially available for swine.

### DESCRIPTION OF THE INVENTION

The solution to the above technical problems is achieved by the description and the embodiments characterized in the claims.

Thus, the invention in its different aspects is implemented according to the claims.

The invention is based on the surprising finding that the administration of a polypeptide comprising a fragment of a rotavirus VP8 protein, namely an N-terminally extended lectin-like domain, being linked at the C-terminus with an IgG Fc fragment, to sows significantly reduced, via passive transmission of neutralizing antibodies, the diarrhea and fecal shedding in their offspring after challenge with rotavirus, even when said polypeptide was mixed, before administration, with additional immunogenic substances.

In a first aspect, the invention thus relates to an immunogenic composition which comprises
(i) a polypeptide comprising
   an immunogenic fragment of a rotavirus VP8 protein, and
   an immunoglobulin Fc fragment,
and
(ii) at least one immunogenic substance, different from said polypeptide,
   wherein said immunogenic composition is also termed “the immunogenic composition of the present invention” hereinafter.

The immunogenic composition of the present invention is thus in particular a composition comprising two components (i; ii), namely
(i) a polypeptide comprising
   an immunogenic fragment of a rotavirus VP8 protein, and
   an immunoglobulin Fc fragment,
wherein said polypeptide is hereinafter also termed “component (i) of the immunogenic composition of the present invention” or “component (i)”, respectively, and
(ii) at least one immunogenic substance, different from said polypeptide,
   wherein said at least one immunogenic substance is hereinafter also termed “component (ii) of the immunogenic composition of the present invention” or “component (ii)”, respectively.

The polypeptide comprising
an immunogenic fragment of a rotavirus VP8 protein, and
an immunoglobulin Fc fragment an immunogenic fragment of a rotavirus VP8 protein,
   which is described herein in the context of the present invention is also termed “the polypeptide of the present disclosure” hereinafter.

Thus, component (i) of the immunogenic composition of the present invention is a polypeptide of the present disclosure.

Preferably, component (ii) consists of two, three or more immunogenic substances, wherein all of said immunogenic substances are
different from component (i) and
different from each other.

In the context of the present invention it has also been unexpectedly discovered that such an immunogenic composition can be simply produced, as the polypeptide of the present disclosure, when produced in cells, is released from the cells, and can then be recovered from the supernatant surrounding the cells rather than from the cells themselves. Subsequently, the recovered polypeptide can be easily mixed with the further component(s), including the at least one immunogenic substance, different from said polypeptide, to produce the immunogenic composition of the present invention.

A further advantage of the polypeptide of the present disclosure is that, if desired, it may be prepared as one polypeptide comprising/presenting two immunogenic fragments of different rotaviruses, thereby making it unnecessary to separately prepare two different monovalent polypeptides which then need to be combined for the same purpose.

Preferably, the immunoglobulin Fc fragment, as described herein, is linked to
the C-terminus of said immunogenic fragment of a rotavirus VP8 protein, or
the N-terminus of said immunogenic fragment of a rotavirus VP8 protein.

In particular, said immunoglobulin Fc fragment is preferably linked to
the C-terminus of said immunogenic fragment of a rotavirus VP8 protein via a linker moiety, or
the N-terminus of said immunogenic fragment of a rotavirus VP8 protein via a linker moiety.

In another preferred aspect, the immunoglobulin Fc fragment, as described herein, is linked to
the C-terminus of said immunogenic fragment of a rotavirus VP8 protein via a peptide bond between the N-terminal amino acid residue of said immunoglobulin Fc fragment and the C-terminal amino acid residue of said immunogenic fragment of a rotavirus VP8 protein, or
the N-terminus of said immunogenic fragment of a rotavirus VP8 protein via a peptide bond between the C-terminal amino acid residue of said immunoglobulin Fc fragment and the N-terminal amino acid residue of said immunogenic fragment of a rotavirus VP8 protein.

Most preferably, the immunoglobulin Fc fragment, as described herein, is linked to the C-terminus of said immunogenic fragment of a rotavirus VP8 protein.

Thus, the polypeptide of the present disclosure is in particular a polypeptide comprising
an immunogenic fragment of a rotavirus VP8 protein, and
an immunoglobulin Fc fragment,
   wherein said immunoglobulin Fc fragment is linked to the C-terminus of said immunogenic fragment of a rotavirus VP8 protein.

The term “polypeptide” used herein in particular refers to any chain of amino acid residues linked together by peptide bonds, and does not refer to a specific length of the product. For instance, “polypeptide” may refer to a long chain of amino acid residues, e.g. one that is 150 to 600 amino acid residues long or longer. The term “polypeptide” includes polypeptides having one or more post-translational modifications, where post-translational modifications include, e.g., glycosylation, phosphorylation, lipidation (e.g., myristoylation, etc.), acetylation, ubiquitylation, sulfation, ADP ribosylation, hydroxylation, Cys/Met oxidation, carboxylation, methylation, etc. The terms “polypeptide” and “protein” are used interchangeably in the context of the present invention.

The term “immunogenic fragment” is in particular understood to refer to a fragment of a protein, which at least partially retains the immunogenicity of the protein from which it is derived. Thus, an “immunogenic fragment of a rotavirus VP8 protein” is particularly understood to refer to a fragment of a rotavirus VP8 protein, which at least partially retains the immunogenicity of the full length VP8 protein.

An “immunogenic substance” refers to any substance capable of eliciting a humoral and/or cellular immune response, in particular to a molecule such as a peptide or polypeptide capable of eliciting, producing, or generating an immune response in an animal.

The term “VP8 protein”, as described herein, is understood to be in particular equivalent to “VP8 domain”, “VP8*” or “VP8 fragment of VP4”, as frequently used in the context of rotavirus.

The term “immunoglobulin Fc fragment”, as used herein, refers to a protein that contains the heavy-chain constant region 2 (CH2) and the heavy-chain constant region 3 (CH3) of an immunoglobulin and, more particular, that does not contain the variable regions of the heavy and light chains, and the light-chain constant region 1 (CL1) of the immunoglobulin. It may further include the hinge region, or a portion of the hinge region, of the immunoglobulin (i.e., the hinge region at the heavy-chain constant region). Also, the immunoglobulin Fc fragment may contain a part or all of the heavy-chain constant region 1 (CH1).

It is understood that the term “immunoglobulin Fc fragment”, as used herein, is equivalent to “immunoglobulin Fc domain”.

The herein used term “linked to” in particular refers to any means for connecting, within a polypeptide, an immunoglobulin Fc fragment to the C-terminus or N-terminus of an immunogenic fragment of a rotavirus VP protein. Examples of linking means include (1.) indirect linkage of the immunoglobulin Fc fragment to the C-terminus of an immunogenic fragment of a rotavirus VP 8 protein by an intervening moiety which is directly linked to the C-terminus of said immunogenic fragment of a rotavirus VP8 protein, and which also binds said immunoglobulin Fc fragment, and (2.) direct linkage of the immunoglobulin Fc fragment to the C-terminus of an immunogenic fragment of a rotavirus VP8 protein by covalent bonding. The terms “linked to” and “linked with” are used interchangeably in the context of the present invention.

It is in particular understood that the wording “polypeptide comprising
an immunogenic fragment of a rotavirus VP8 protein, and
an immunoglobulin Fc fragment,
   wherein said immunoglobulin Fc fragment is linked to the C-terminus of said immunogenic fragment of a rotavirus VP8 protein”,
   as used herein, is in particular equivalent to the wording
   “polypeptide comprising, in N- to C-terminal direction,
the amino acid sequence of an immunogenic fragment of a rotavirus VP8 protein, and
the amino acid sequence of an immunoglobulin Fc fragment”,
   or to the wording
   “polypeptide comprising
an immunogenic fragment of a rotavirus VP8 protein, and
an immunoglobulin Fc fragment linked to the C-terminus of said immunogenic fragment”.

According to a most preferred aspect, the immunoglobulin Fc fragment is linked to the C-terminus of said immunogenic fragment of a rotavirus VP8 protein via a linker moiety.

The linker moiety, as described herein in the context of the present invention, is preferably a peptide linker.

The term “peptide linker” as used herein refers to a peptide comprising one or more amino acid residues. More particular, the term “peptide linker” as used herein refers to a peptide capable of connecting two variable proteins and/or domains, e.g. an immunogenic fragment of a rotavirus VP8 protein and an immunoglobulin Fc fragment.

In a particular preferred aspect, the immunoglobulin Fc fragment is linked to the C-terminus of said immunogenic fragment of a rotavirus VP8 protein via a linker moiety, wherein
the immunogenic fragment of a rotavirus VP8 protein is linked to the linker moiety via a peptide bond between the N-terminal amino acid residue of the linker moiety and the C-terminal amino acid residue of the immunogenic fragment of a rotavirus VP8 protein, and
the linker moiety is linked to the immunoglobulin Fc fragment via a peptide bond between the N-terminal amino acid residue of the immunoglobulin Fc fragment and the C-terminal amino acid residue of the linker moiety.

Also, it may be preferred that the immunoglobulin Fc fragment is linked to the immunogenic fragment of a rotavirus VP8 protein via a peptide bond between the N-terminal amino acid residue of the immunoglobulin Fc fragment and the C-terminal amino acid residue of the immunogenic fragment of a rotavirus VP8 protein.

It will be understood that the polypeptide of the present disclosure is in particular a fusion protein.

As used herein the term “fusion protein” means a protein formed by fusing (i.e., joining) all or part of two or more polypeptides which are not the same. Typically, fusion proteins are made using recombinant DNA techniques, by end to end joining of polynucleotides encoding the two or more polypeptides. More particular, the term “fusion protein” thus refers to a protein translated from a nucleic acid transcript generated by combining a first nucleic acid sequence that encodes a first polypeptide and at least a second nucleic acid that encodes a second polypeptide, where the fusion protein is not a naturally occurring protein. The nucleic acid construct may encode two or more polypeptides that are joined in the fusion protein.

In another preferred aspect, the polypeptide of the present disclosure is a fusion protein of the formula x-y-z, wherein
x consists of or comprises an immunogenic fragment of a rotavirus VP8 protein;
y is a linker moiety; and
z is an immunoglobulin Fc fragment.

The formula x-y-z is in particular to be understood that the C-terminal amino acid residue of said immunogenic fragment of a rotavirus VP8 protein is linked with said linker moiety, preferably via a peptide bond with the N-terminal amino acid residue of said linker moiety, and that the N-terminal amino acid residue of said immunoglobulin Fc fragment is linked with said linker moiety, preferably via a peptide bond with the C-terminal amino acid residue of said linker moiety.

The wording “x consists of an immunogenic fragment of a rotavirus VP8 protein”, as described herein, is in particular understood to be equivalent to “x is an immunogenic fragment of a rotavirus VP8 protein”.

In a preferred aspect, the immunogenic fragment of a rotavirus VP8 protein, as mentioned herein, is preferably capable of inducing an immune response against rotavirus in a subject to whom said immunogenic fragment of a rotavirus VP8 protein is administered.

In another preferred aspect, the immunogenic fragment of a rotavirus VP8 protein is a polypeptide being 50 to 200, preferably 140 to 190 amino acid residues, in length.

The rotavirus mentioned herein is preferably selected from the group consisting of rotavirus A and rotavirus C. Hence, the immunogenic fragment of a rotavirus VP8 protein, as mentioned herein, is preferably selected from the group consisting of immunogenic fragment of a rotavirus A VP8 protein and immunogenic fragment of a rotavirus C VP8 protein.

The term(s) “rotavirus A” and “rotavirus C”, respectively, as mentioned herein, relate(s) to rotavirus A and rotavirus C, respectively, as defined by the ICTV (summarized by Matthijnssens et al. Arch Virol 157:1177-1182 (2012)).

According to another preferred aspect, the rotavirus mentioned herein is a porcine rotavirus.

In one particularly preferred aspect, the rotavirus mentioned herein is rotavirus A. Thus, the immunogenic fragment of a rotavirus VP8 protein, as described herein, is preferably an immunogenic fragment of a rotavirus A VP8 protein.

In a further preferred aspect, the immunogenic fragment of a rotavirus VP8 protein comprises the lectin-like domain of a rotavirus VP8 protein. The “lectin-like domain of a rotavirus VP8 protein”, as mentioned herein, is understood to be preferably a lectin-like domain of a rotavirus A VP8 protein.

The term “lectin-like domain of a rotavirus VP8 protein” in particular refers to residues 65-224 of a rotavirus VP8 protein or, respectively, corresponds to the amino acid sequence consisting of the amino acid residues 65-224 of a rotavirus VP8 protein, and wherein said amino acid residues 65-224 of a rotavirus VP8 protein are preferably the amino acid residues 65-224 of a rotavirus A VP8 protein.

Thus, the “lectin-like domain of a rotavirus VP8 protein” preferably consists of the amino acid sequence of the amino acid residues 65-224 of a rotavirus VP8 protein, in particular of a rotavirus A VP8 protein.

Preferably, the immunogenic fragment of a rotavirus VP8 protein is an N-terminally extended lectin-like domain of a rotavirus VP8 protein, wherein the N-terminal extension is 1 to 20 amino acid residues, in particular 5 to 15 amino acid residues, in length. Most preferably, the immunogenic fragment of a rotavirus VP8 protein is an N-terminally extended lectin-like domain of a rotavirus VP8 protein, wherein the N-terminal extension is eight amino acid residues in length.

The amino acid sequence of said N-terminal extension is preferably the amino acid sequence of the respective length flanking the N-terminal amino acid residue of the lectin-like domain in the amino acid sequence of the rotavirus VP8 protein.

Thus, in a particular aspect, the immunogenic fragment of a rotavirus VP8 protein, as mentioned herein, preferably consists of the amino acid sequence of the amino acid residues 60-224, the amino acid residues 59-224, the amino acid residues 58-224, the amino acid residues 57-224, the amino acid residues 56-224, the amino acid residues 55-224, the amino acid residues 54-224, the amino acid residues 53-224, the amino acid residues 52-224, the amino acid residues 51-224, the amino acid residues 50-224, or the amino residues 49-224, of a rotavirus VP8 protein, in particular of a rotavirus A protein.

Most preferably, the immunogenic fragment of a rotavirus VP8 protein, as mentioned herein, consists of the amino acid sequence of the amino acid residues 57-224 of a rotavirus VP8 protein, in particular of a rotavirus A protein.

The above numbering of amino acid residues (e.g. “65-224” or “57-224”) is preferably with reference to the amino acid sequence of a wild-type rotavirus VP8 protein, in particular of a wild-type rotavirus A VP8 protein. Said wild-type rotavirus VP8 protein is preferably the protein set forth in SEQ ID NO:1.

According to a further preferred aspect, the rotavirus mentioned herein is a rotavirus, in particular a rotavirus A, selected from the group consisting of genotype P[6] rotavirus, genotype P[7] rotavirus and genotype P[13] rotavirus. Thus, the immunogenic fragment of a rotavirus VP8 protein, as mentioned herein, is preferably selected from the group consisting of immunogenic fragment of a genotype P[6] rotavirus VP8 protein, immunogenic fragment of a genotype P[7] rotavirus VP8 protein and immunogenic fragment of a genotype P[13] rotavirus VP8 protein, and is in particular selected from the group consisting of immunogenic fragment of a genotype P[6] rotavirus A VP8 protein, immunogenic fragment of a genotype P[7] rotavirus A VP8 protein and immunogenic fragment of a genotype P[13] rotavirus A VP8 protein.

The terms “genotype P[6] rotavirus”, “genotype P[7] rotavirus”, “genotype P[13] rotavirus” and “genotype P[23] rotavirus”, as used herein, in particular relate to the established VP4 (P) genotype classification of rotaviruses (e.g., P[6], P[7], P[13] or P[23]) which is described in: Estes and Kapikian. Rotaviruses. In: Knipe, D. M.; Howley, P. M. Fields Virology, 5th ed.; Wolters Kluwer/Lippincott Williams & Wilkins Health: Philadelphia, PA, USA (2007); Gorziglia et al. Proc Natl Acad Sci USA. 87(18):7155-9 (1990).

Most preferably, the rotavirus mentioned herein is a genotype P[7] rotavirus. Thus, the immunogenic fragment of a rotavirus VP8 protein, as mentioned herein, is most preferably an immunogenic fragment of a genotype P[7] rotavirus VP8 protein, in particular an immunogenic fragment of a genotype P[7] rotavirus A VP8 protein.

The rotavirus VP8 protein mentioned herein comprises or consists of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with the amino acid sequence of SEQ ID NO:1.

The lectin-like domain of a rotavirus VP8 protein, as mentioned herein, comprises or consists of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with the amino acid sequence of SEQ ID NO:2.

In one example, the immunogenic fragment of a rotavirus VP8 protein consists of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with the amino acid sequence of SEQ ID NO:3.

In another preferred aspect, the immunogenic fragment of a rotavirus VP8 protein consists of or is a consensus sequence of a portion of a rotavirus VP8 protein, in particular of a portion of a rotavirus A VP8 protein.

As used herein, the term “consensus sequence” in particular refers to the sequence formed from the most frequently occurring amino acids (or nucleotides) in a family of related sequences (See e.g., Winnaker, From Genes to Clones (Verlagsgesellschaft, Weinheim, Germany 1987)). In a family of proteins, each position in the consensus sequence is occupied by the amino acid occurring most frequently at that position in the family. The term “consensus sequence” thus stands for a deduced amino acid sequence (or nucleotide sequence). The consensus sequence represents a plurality of similar sequences. Each position in the consensus sequence corresponds to the most frequently occurring amino acid residue (or nucleotide base) at that position which is determined by aligning three or more sequences.

Preferably, a consensus sequence of a portion of a rotavirus VP8 protein, as mentioned herein, is obtainable by a method comprising the steps of:
translating a plurality of nucleotide sequences encoding a portion of a rotavirus VP8 protein into amino acid sequences,
aligning said amino acid sequences to known rotavirus VP8 proteins, preferably by using MUSCLE sequence alignment software UPGMB clustering and default gap penalty parameters,
subjecting said aligned sequences to a phylogenetic analysis and generating a neighbor joining phylogeny reconstruction based on rotavirus VP8 protein sequence, in particular importing said aligned amino acid sequences into MEGA7 software for phylogenetic analysis and generating a neighbor joining phylogeny reconstruction based on rotavirus VP8 protein sequence,
computing the optimal tree using the Poisson correction method with bootstrap test of phylogeny (n=100),
drawing the optimal tree to scale with branch lengths equal to evolutionary distances in units of amino acid substitutions per site over 170 total positions,
considering nodes where bootstrap cluster association is greater than 70% as significant,
designating nodes with approximately 10% distance and bootstrap cluster associations greater than 70% as clusters, and
selecting a cluster and generating the consensus sequences by identifying the greatest frequency per aligned position within the cluster,
and optionally, in cases where equivalent proportions of amino acids are observed in an aligned position, selecting the amino acid residue based on reported epidemiological data in conjunction with a predefined product protection profile.

For example, in this context, the immunogenic fragment of a rotavirus VP8 protein comprises or consists of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with a sequence selected from the group consisting of SEQ ID NO:4 and SEQ ID NO:5.

In a further preferred aspect, the rotavirus mentioned herein is rotavirus C. According to this aspect, the immunogenic fragment of a rotavirus VP8 protein is preferably an immunogenic fragment of a rotavirus C VP8 protein.

In the context of this aspect, the immunogenic fragment of a rotavirus VP8 protein comprises or consists of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with the amino acid sequence of SEQ ID NO:6.

According to the present invention, the immunogenic fragment of a rotavirus VP8 protein thus preferably consists of or is
an immunogenic fragment of a rotavirus A VP8 protein, in particular any of the herein described immunogenic fragments of a rotavirus A VP8 protein, or
a consensus sequence of a portion of a rotavirus VP8 protein, such as of a portion of a rotavirus A VP8 protein, preferably any of the immunogenic fragments of a rotavirus VP8 protein described herein in the context of a consensus sequence, or
an immunogenic fragment of a rotavirus C VP8 protein, in particular any of the herein described immunogenic fragments of a rotavirus C VP8 protein.

In a particular preferred aspect, the immunogenic fragment of a rotavirus VP8 protein is a polypeptide comprising or consisting of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with a sequence selected from the group consisting of SEQ ID NO:3, SEQ ID NO:4, SEQ ID NO:5 and SEQ ID NO:6.

The immunoglobulin Fc fragment described herein is preferably at least 220 amino acid residues in length, and most preferably 220 to 250 amino acid residues in length.

According to another particular preferred aspect, the herein described immunoglobulin Fc fragment is non-glycosylated. The term “non-glycosylated”, as used herein, in particular means that the immunoglobulin Fc fragment does not have oligosaccharide molecules attached thereto.

Preferably, the immunoglobulin Fc fragment, as mentioned herein, comprises or consists of
the heavy-chain constant region 2 (CH2), and
the heavy-chain constant region 3 (CH3),
and optionally the hinge region or a portion of the hinge region,
   of an immunoglobulin.

According to another preferred aspect, the immunoglobulin mentioned herein is selected from the group consisting of IgG, IgA, IgD, IgE and IgM. Thus, the immunoglobulin Fc fragment is preferably selected from the group consisting of IgG Fc fragment, IgA Fc fragment, IgD Fc fragment, IgE Fc fragment and IgM Fc fragment.

According to a most preferred aspect, the immunoglobulin Fc fragment described herein is an IgG Fc fragment.

The IgG, as mentioned herein, is preferably selected from the group consisting of IgG1, IgG2, IgG3, IgG4, IgG5 and IgG6. Thus, according to another preferred aspect, the herein mentioned immunoglobulin Fc fragment is selected from the group consisting of IgG1 Fc fragment, IgG2 Fc fragment, IgG3 Fc fragment, IgG4 Fc fragment, IgG5 Fc fragment and IgG6 Fc fragment.

Most preferably, the immunoglobulin Fc fragment is a protein encoded by the genome of a species whose intestinal cells are susceptible to an infection by the rotavirus from which the immunogenic fragment of a rotavirus VP8 protein, as mentioned herein, is derived. If, for example, the fragment of a rotavirus VP8 protein is the fragment of a porcine rotavirus VP8 protein, then the immunoglobulin Fc fragment is preferably an immunoglobulin Fc fragment encoded by a porcine genome. According to another example, if the fragment of a rotavirus VP8 protein is the fragment of a chicken rotavirus VP8 protein, then the immunoglobulin Fc fragment is preferably an immunoglobulin Fc fragment encoded by a chicken genome.

More particular, the immunoglobulin Fc fragment preferably is a swine IgG Fc fragment.

In a further preferred aspect, the immunoglobulin Fc fragment comprises or consists of an amino acid sequence having at least 70%, at least 80%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with a sequence selected from the group consisting of SEQ ID NO:7 and SEQ ID NO:8.

The linker moiety, or peptide linker, respectively, mentioned herein is preferably an amino acid sequence being 1 to 50 amino acid residues in length, in particular being 3 to 20 amino acid residues in length. For example, the linker moiety may be a peptide linker being 3, 8 or 10 amino acid residues in length.

Depending on the purpose, a short linker may be desired to decrease the risk of proteolysis between the fusion protein partners. Thus, the peptide linker described in the context of the present invention preferably has a length, or consists, respectively, of 1-5 amino acid residues, more preferably 2-4 amino acid residues and most preferably three amino acid residues.

According to a preferred aspect, the linker moiety comprises or consists of an amino acid sequence having at least 66%, at least 80%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with a sequence selected from the group consisting of SEQ ID NO:9, SEQ ID NO:10 and SEQ ID NO:11.

“SEQ ID NO:9”, as mentioned herein, refers to the amino acid sequence (in single letter code for amino acid residues): GGS, which in conventional three-letter code is Gly-Gly-Ser.

Thus, “SEQ ID NO:9” in the context of the present invention represents the amino acid sequence Gly-Gly-Ser or, respectively, “Gly Gly Ser” as set out, for the sequence of SEQ ID NO:9, in the sequence listing below.

Preferably, the polypeptide of the present disclosure has an N-terminal methionine residue flanking the N-terminal amino acid residue of the immunogenic fragment of a rotavirus VP8 protein.

According to another preferred aspect, the polypeptide of the present disclosure comprises a further immunogenic fragment of a rotavirus VP8 protein linked to the C-terminus of said immunoglobulin Fc fragment.

Said further immunogenic fragment of a rotavirus VP8 protein preferably consists of or is
an immunogenic fragment of a rotavirus A VP8 protein, in particular any of the herein described immunogenic fragments of a rotavirus A VP8 protein, or
a consensus sequence of a portion of a rotavirus VP8 protein, such as of a portion of a rotavirus A VP8 protein, preferably any of the immunogenic fragments of a rotavirus VP8 protein described herein in the context of a consensus sequence, or
an immunogenic fragment of a rotavirus C VP8 protein, in particular any of the herein described immunogenic fragments of a rotavirus C VP8 protein.

In particular, said further immunogenic fragment of a rotavirus VP8 protein preferably comprises or consists of an amino acid sequence having at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with a sequence selected from the group consisting of SEQ ID NOs: 2 to 6.

In a particular preferred aspect, said further immunogenic fragment of a rotavirus VP8 protein is different from the immunogenic fragment of a rotavirus VP8 protein of which the C-terminus is linked to said immunoglobulin Fc fragment.

Said further immunogenic fragment of a rotavirus VP8 protein is preferably linked to the C-terminus of said immunoglobulin Fc fragment via a linker moiety, in particular via any of the linker moieties described herein. Preferably, said further immunogenic fragment of a rotavirus VP8 protein is linked to the linker moiety via a peptide bond between the N-terminal amino acid residue of said further immunogenic fragment of a rotavirus VP8 protein and the C-terminal amino acid residue of the linker moiety.

Alternatively, it may be preferred that said further immunogenic fragment of a rotavirus VP8 protein is linked to the C-terminus of said immunoglobulin Fc fragment via a peptide bond between the N-terminal amino acid residue of said further immunogenic fragment of a rotavirus VP8 protein and the C-terminal amino acid residue of said immunoglobulin Fc fragment.

In a particular preferred aspect, the polypeptide of the present disclosure is a protein comprising or consisting of an amino acid sequence having at least 70%, at least 80%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity with a sequence selected from the group consisting of SEQ ID NO:12, SEQ ID NO:13, SEQ ID NO:14, SEQ ID NO:15 and SEQ ID NO:16.

Preferably, the polypeptide of the present disclosure is a protein comprising or consisting of an amino acid sequence selected from the group consisting of SEQ ID NO:12, SEQ ID NO:13, SEQ ID NO:14, SEQ ID NO:15 and SEQ ID NO:16.

It is understood that the wording “consisting of an amino acid sequence” or “consists of an amino acid sequence”, respectively, as used herein, in particular also concerns any cotranslational and/or posttranslational modification or modifications of the amino sequence affected by the cell in which the protein or protein domain is expressed. Thus, the wording “consisting of an amino acid sequence” or “consists of an amino acid sequence”, respectively, as described herein, is also directed, unless expressly mentioned otherwise, to the amino acid sequence having one or more modifications effected by the cell in which the protein or protein domain is expressed, in particular modifications of amino acid residues effected in the protein biosynthesis and/or protein processing, preferably selected from the group consisting of glycosylations, phosphorylations, and acetylations.

Regarding the term “at least 90%”, as mentioned in the context of the present invention, it is understood that said term preferably relates to “at least 91%”, more preferably to “at least 92%”, still more preferably to “at least 93%” or in particular to “at least 94%”.

Regarding the term “at least 95%” as mentioned in the context of the present invention, it is understood that said term preferably relates to “at least 96%”, more preferably to “at least 97%”, still more preferably to “at least 98%” or in particular to “at least 99%”.

Regarding the term “at least 99%” as mentioned in the context of the present invention, it is understood that said term preferably relates to “at least 99.2%”, more preferably to “at least 99.4%”, still more preferably to “at least 99.6%” or in particular to “at least 99.8%”.

More particular, the term “at least 99% sequence identity” refers to 99.1%, 99.2%, 99.3%, 99.4%, 99.5%, 99.6%, 99.7%, 99.8%, or 99.9% sequence identity.

The term “having 100% sequence identity”, as used herein, is understood to be equivalent to the term “being identical”.

Percent sequence identity has an art recognized meaning and there are a number of methods to measure identity between two polypeptide or polynucleotide sequences. See, e.g., Lesk, Ed., Computational Molecular Biology, Oxford University Press, New York, (1988); Smith, Ed., Biocomputing: Informatics And Genome Projects, Academic Press, New York, (1993); Griffin & Griffin, Eds., Computer Analysis Of Sequence Data, Part I, Humana Press, New Jersey, (1994); von Heinje, Sequence Analysis In Molecular Biology, Academic Press, (1987); and Gribskov & Devereux, Eds., Sequence Analysis Primer, M Stockton Press, New York, (1991). Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Molec. Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. App. Math., 2:482-489 (1981)). For example, the computer program ALIGN which employs the FASTA algorithm can be used, with an affine gap search with a gap open penalty of −12 and a gap extension penalty of −2. For purposes of the present invention, nucleotide sequences are aligned using Clustal W method in MegAlign software version 11.1.0 (59), 419 by DNASTAR Inc. using the default multiple alignment parameters set in the program (Gap penalty=15.0, gap length penalty=6.66, delay divergent sequence (%)=30%, DNA transition weight=0.50 and DNA weight matrix=IUB) and, respectively, protein/amino acid sequences are aligned using Clustal W method in MegAlign software version 11.1.0 (59), 419 by DNASTAR Inc. using the default multiple alignment parameters set in the program (Gonnet series protein weight matrix with Gap penalty=10.0, gap length penalty=0.2, and delay divergent sequence (%)=30%).

As used herein, it is in particular understood that the term “sequence identity with the sequence of SEQ ID NO:X” is equivalent to the term “sequence identity with the sequence of SEQ ID NO:X over the length of SEQ ID NO:X” or to the term “sequence identity with the sequence of SEQ ID NO:X over the whole length of SEQ ID NO:X”, respectively. In this context, “X” is any integer selected from 1 to 25 so that “SEQ ID NO:X” represents any of the SEQ ID NOs mentioned herein.

The wording “group consisting of SEQ ID NO:[ . . . ], . . . and SEQ ID NO:[ . . . ]”, as used herein, is interchangeable to “group consisting of: the sequence of SEQ ID NO:[ . . . ], . . . and the sequence of SEQ ID NO:[ . . . ]”. “[ . . . ]” in this context is a placeholder for the number of the sequence. For instance, the wording “group consisting of SEQ ID NO:3, SEQ ID NO:4, SEQ ID NO:5 and SEQ ID NO:6” is interchangeable to “group consisting of: the sequence of SEQ ID NO:3, the sequence of SEQ ID NO:4, the sequence of SEQ ID NO:5 and the sequence of SEQ ID NO:6”.

According to another particular preferred aspect, the polypeptide of the present disclosure consists of:
an immunogenic fragment of a rotavirus VP8 protein, in particular any of the herein described immunogenic fragments of a rotavirus VP8 protein,
an N-terminal methionine residue flanking the N-terminal amino acid residue of said immunogenic fragment of a rotavirus VP8 protein, and
an immunoglobulin Fc fragment, in particular any of the herein described immunoglobulin Fc fragments,
wherein said immunoglobulin Fc fragment is linked to the C-terminus of said immunogenic fragment of a rotavirus VP8 protein, in particular via a linker moiety, wherein said linker moiety is preferably any of the herein described linker moieties,
and optionally a further immunogenic fragment of a rotavirus VP8 protein linked to the C-terminus of said immunoglobulin Fc fragment, in particular via a linker moiety, wherein said further immunogenic fragment of a rotavirus VP8 protein is preferably any of the herein described further immunogenic fragments of a rotavirus VP8 protein, and wherein said linker moiety is preferably any of the herein described linker moieties.

In a yet further preferred aspect, the polypeptide of the present disclosure forms a dimer with a further polypeptide of the present disclosure. Most preferably, the polypeptide of the present disclosure forms a homodimer with a further identical polypeptide.

It is thus particularly understood that the term “polypeptide of the present disclosure” further encompasses any dimer composed of two polypeptides of the present disclosure, and in particular encompasses any homodimer composed of two identical polypeptides of the present disclosure.

According to another particular preferred aspect, the present disclosure provides a multimer comprising or composed of a plurality of the polypeptide of the present disclosure, and wherein said multimer is also termed “the multimer of the present disclosure” hereinafter.

Preferably, the multimer of the present disclosure is a homodimer formed by one polypeptide of the present disclosure with a further identical polypeptide of the present disclosure.

It is in particular understood, that the term “multimer of the present disclosure” further encompasses any mixture of different multimers of the present disclosure, e.g. a mixture of
a homodimer formed by one polypeptide of the present disclosure with a further identical polypeptide of the present disclosure, and
one or more multimers formed by more than two of the same polypeptides of the present disclosure.

Thus, in one particularly preferred example, component (i) of the immunogenic composition of the present invention is present
in a monomer consisting of one polypeptide of the present disclosure, and/or
a homodimer consisting of two identical polypeptides of the present disclosure,
and/or optionally a homotrimer consisting of three identical polypeptides of the present disclosure,
   wherein preferably
each of said two identical polypeptides of the present disclosure,
and optionally each of said three identical polypeptides of the present disclosure,
   comprises or consists of the same amino acid sequence as said one polypeptide of the present disclosure.

Or, more generally, the present invention also encompasses an immunogenic composition which comprises
(i) a polypeptide comprising
   an immunogenic fragment of a rotavirus VP8 protein, and
   an immunoglobulin Fc fragment,
wherein optionally said polypeptide is included in a multimer comprising or composed of a plurality of said polypeptide,
   and
(ii) at least one immunogenic substance, different from said polypeptide.

The herein described at least one immunogenic substance, different from said polypeptide, preferably consists of two or more immunogenic substances, wherein all of said immunogenic substances are
different from said polypeptide and
different from each other.

Thus, component (ii) of the immunogenic composition of the present invention preferably consists of two or more immunogenic substances, wherein each of said immunogenic substances is different from component (i) of the immunogenic composition of the present invention, and wherein all of said immunogenic substances are different from each other.

Preferably, the at least one immunogenic substance, different from said polypeptide, is at least one immunogenic substance which comprises or consists of a reovirus antigen, different from said immunogenic fragment. Thus, component (ii) is preferably at least one immunogenic substance comprising or consisting of a reovirus antigen, different from said immunogenic fragment of component (i).

As used herein, the term “reovirus antigen” in particular refers to a peptide or protein, or a part thereof, comprising an epitope that is recognized by an antibody specific for a virus belonging to the family Reoviridae.

In one preferred aspect, the at least one immunogenic substance, different from said polypeptide, consists of two or more immunogenic substances, wherein each of said substances comprises or consists of a reovirus antigen, and wherein all of said antigens are different from said immunogenic fragment and different from each other. Thus, component (ii) preferably consists of two or more immunogenic substances, wherein each of said substances comprises or consists of a reovirus antigen, and wherein all of said antigens are different from said immunogenic fragment of component (i) and are different from each other.

In another preferred aspect, the at least one immunogenic substance, different from said polypeptide, is at least one protein which comprises or consists of a reovirus antigen, different from said immunogenic fragment. Thus, component (ii) is preferably at least one protein comprising or consisting of a reovirus antigen, different from said immunogenic fragment of component (i).

In still a further preferred aspect, the at least one immunogenic substance, different from said polypeptide, consists of two or more proteins, wherein each of said proteins comprises or consists of a reovirus antigen, and wherein all of said reovirus antigens are different from said immunogenic fragment and different from each other. Thus, component (ii) preferably consists of two or more proteins, wherein each of said proteins comprises or consists of a reovirus antigen, and wherein all of said antigens are different from said immunogenic fragment of component (i) and are different from each other.

Preferably, the at least one immunogenic substance, different from said polypeptide, is at least one immunogenic substance which comprises a rotavirus antigen, different from said immunogenic fragment. Thus, component (ii) is preferably at least one immunogenic substance comprising a rotavirus antigen, different from said immunogenic fragment of component (i).

The term “rotavirus antigen”, as used herein, in particular refers to a peptide or protein, or a part thereof, comprising an epitope that is recognized by an antibody specific for a virus belonging to the genus Rotavirus.

In one preferred aspect, the at least one immunogenic substance, different from said polypeptide, consists of two or more immunogenic substances, wherein each of said substances comprises or consists of a rotavirus antigen, and wherein all of said antigens are different from said immunogenic fragment and different from each other. Thus, component (ii) preferably consists of two or more immunogenic substances, wherein each of said substances comprises or consists of a rotavirus antigen, and wherein all of said antigens are different from said immunogenic fragment of component (i) and are different from each other.

In another preferred aspect, the at least one immunogenic substance, different from said polypeptide, is at least one protein which comprises or consists of a rotavirus antigen, different from said immunogenic fragment. Thus, component (ii) is preferably at least one protein comprising or consisting of a rotavirus antigen, different from said immunogenic fragment of component (i).

In still a further preferred aspect, the at least one immunogenic substance, different from said polypeptide, consists of two or more proteins, wherein each of said proteins comprises or consists of a rotavirus antigen, and wherein all of said rotavirus antigens are different from said immunogenic fragment and different from each other. Thus, component (ii) preferably consists of two or more proteins, wherein each of said proteins comprises or consists of a rotavirus antigen, and wherein all of said antigens are different from said immunogenic fragment of component (i) and are different from each other.

In a particularly preferred aspect, the immunogenic composition of the present invention is preferably an immunogenic composition, where in
(i) said polypeptide is a first polypeptide;
and
(ii) the at least one immunogenic substance, different from said polypeptide, is at least one additional polypeptide, different from said first polypeptide.

Preferably, said at least one immunogenic substance comprises or is
a second polypeptide which comprises or consists of a reovirus antigen, different from said immunogenic fragment,
   and optionally comprises a third polypeptide, different from both said first and second polypeptide,
   and optionally comprises a fourth polypeptide, different from all of said first to third polypeptides.

Preferably, said third polypeptide comprises a reovirus antigen, different from both said immunogenic fragment and reovirus antigen of the second polypeptide.

Said fourth polypeptide preferably comprises a reovirus antigen, different from all of
said immunogenic fragment,
reovirus antigen of the second polypeptide and
reovirus antigen of the third polypeptide.

In another particularly preferred aspect,
component (i) of the immunogenic composition of the present invention is a first polypeptide
and component (ii) of the immunogenic composition of the present invention is at least one additional polypeptide, different from said first polypeptide,
and wherein preferably component (ii)
   comprises or is a second polypeptide which comprises a rotavirus antigen, different from said immunogenic fragment of component (i), and
   preferably comprises a third polypeptide, different from both said first and second polypeptide, and
   optionally comprises a fourth polypeptide, different from all of said first to third polypeptides.

Preferably, said third polypeptide comprises a rotavirus antigen, different from both said immunogenic fragment of component (i) and rotavirus antigen of the second polypeptide.

According to a preferred aspect, said second polypeptide is any of the polypeptides of the present disclosure, as described herein, provided that said second polypeptide is different from said first polypeptide,
and preferably said third polypeptide is any of the polypeptides of the present disclosure, as described herein, provided that said third polypeptide is different from both said first and second polypeptide,
and optionally said fourth polypeptide is any of the polypeptides of the present disclosure, as described herein, provided that said fourth polypeptide is different from all of said first to third polypeptides.

In another preferred aspect, the immunogenic composition of the present invention is preferably an immunogenic composition, where in
(i) said polypeptide is a first polypeptide comprising
   a first immunogenic fragment of a rotavirus VP8 protein, and
   an immunoglobulin Fc fragment;
and
(ii) the at least one immunogenic substance, different from said polypeptide, comprises or consists of
   a second polypeptide comprising a second immunogenic fragment of a rotavirus VP8 protein, wherein said second immunogenic fragment is different from said first immunogenic fragment,
   and preferably a third polypeptide comprising a third immunogenic fragment of a rotavirus VP8 protein, wherein said third immunogenic fragment is different from both said first and second immunogenic fragment,
   and optionally a fourth polypeptide comprising a fourth immunogenic fragment of a rotavirus VP8 protein, wherein said fourth immunogenic fragment is different from all of said first to third immunogenic fragments.

Preferably, each of said second to fourth immunogenic fragments is individually selected from the group consisting of
an immunogenic fragment of a rotavirus A VP8 protein, in particular any of the herein described immunogenic fragments of a rotavirus A VP8 protein,
a consensus sequence of a portion of a rotavirus VP8 protein, such as of a portion of a rotavirus A VP8 protein, preferably any of the immunogenic fragments of a rotavirus VP8 protein described herein in the context of a consensus sequence, and
an immunogenic fragment of a rotavirus C VP8 protein, in particular any of the herein described immunogenic fragments of a rotavirus C VP8 protein,
   in particular provided that all of said second to fourth immunogenic fragments are different from said first immunogenic fragment and different from each other.

More specifically,
said first immunogenic fragment is selected from the group consisting of X7, X13, X6 and XC, and/or
said second immunogenic fragment is selected from the group consisting X7, X13, X6 and XC, and/or
said third immunogenic fragment is selected from the group consisting of X7, X13, X6 and XC, and/or
said fourth immunogenic fragments is selected from the group consisting of X7, X13, X6 and XC;
wherein
X7 represents an immunogenic fragment of a genotype P[7] rotavirus VP8 protein,
X13 represents an immunogenic fragment of a genotype P[13] rotavirus VP8 protein,
X6 represents an immunogenic fragment of a genotype P[6] rotavirus VP8 protein, and
XC represents an immunogenic fragment of a rotavirus C VP8 protein,
   in particular provided that all of said second to fourth immunogenic fragments are different from said first immunogenic fragment and different from each other.

Thus, as referred to herein,
“X7” means an immunogenic fragment of a genotype P[7] rotavirus VP8 protein,
“X13” means an immunogenic fragment of a genotype P[13] rotavirus VP8 protein,
“X6” means an immunogenic fragment of a genotype P[6] rotavirus VP8 protein, and
“XC” means an immunogenic fragment of a rotavirus C VP8 protein.

X7, as referred to herein, preferably consists of an amino acid sequence having at least 90%, preferably at least 95%, more preferably at least 98% or still more preferably at least 99% sequence identity with the sequence of SEQ ID NO:3.

X13, as referred to herein, preferably consists of an amino acid sequence having at least 90%, preferably at least 95%, more preferably at least 98% or still more preferably at least 99% sequence identity with the sequence of SEQ ID NO:5.

X6, as referred to herein, preferably consists of an amino acid sequence having at least 90%, preferably at least 95%, more preferably at least 98% or still more preferably at least 99% sequence identity with the sequence of SEQ ID NO:4.

XC, as referred to herein, preferably consists of an amino acid sequence having at least 90%, preferably at least 95%, more preferably at least 98% or still more preferably at least 99% sequence identity with the sequence of SEQ ID NO:6.

In particular, the at least one immunogenic substance, different from said polypeptide, comprises or consists of a second polypeptide comprising a second immunogenic fragment of a rotavirus VP8 protein, and
each of said first and second immunogenic fragment is individually selected from the group consisting of X7, X13, X6 and XC,
provided that when said first immunogenic fragment is X7, then said second immunogenic fragment is selected from the group consisting of X13, X6 and XC,
provided that when said first immunogenic fragment is X6, then said second immunogenic fragment is selected from the group consisting of X7, X13 and XC,
provided that when said first immunogenic fragment is X13, then said second immunogenic fragment is selected from the group consisting of X7, X6 and XC,
and provided that when said first immunogenic fragment is XC, then said second immunogenic fragment is selected from the group consisting of X7, X13 and X6.

More particular, the at least one immunogenic substance, different from said polypeptide, comprises or consists of
a second polypeptide comprising a second immunogenic fragment of a rotavirus VP8 protein, and
a third polypeptide comprising a third immunogenic fragment of a rotavirus VP8 protein,
and each of said first to third immunogenic fragments is individually selected from the group consisting of X7, X13, X6 and XC,
provided that when said first immunogenic fragment is X7, then said second immunogenic fragment is X13 and said third immunogenic fragment is selected from the group consisting of X6 and XC,
provided that when said first immunogenic fragment is X6, then said second immunogenic fragment is X7 and said third immunogenic fragment is selected from the group consisting of X13 and XC,
provided that when said first immunogenic fragment is X13, then said second immunogenic fragment is X7 and said third immunogenic fragment is selected from the group consisting of X6 and XC,
and provided that when said first immunogenic fragment is XC, then said second immunogenic fragment is X7 and said third immunogenic fragment is selected from the group consisting of X13 and X6.

Still more particular, the at least one immunogenic substance, different from said polypeptide, comprises or consists of
a second polypeptide comprising a second immunogenic fragment of a rotavirus VP8 protein, and
a third polypeptide comprising a third immunogenic fragment of a rotavirus VP8 protein, and
a fourth polypeptide comprising a fourth immunogenic fragment of a rotavirus VP8 protein,
wherein each of said first to fourth immunogenic fragments is individually selected from the group consisting of X7, X6, X13 and XC,
provided that when said first immunogenic fragment is X7, then said second immunogenic fragment is X13, said third immunogenic fragment is X6 and said fourth immunogenic fragment is XC,
provided that when said first immunogenic fragment is X6, then said second immunogenic fragment is X7, said third immunogenic fragment is X13 and said fourth immunogenic fragment is XC,
provided that when said first immunogenic fragment is X13, then said second immunogenic fragment is X7, said third immunogenic fragment is X6 and said fourth immunogenic fragment is XC,
and provided that when said first immunogenic fragment is XC, then said second immunogenic fragment is X7, said third immunogenic fragment is X13 and said fourth immunogenic fragment is X6.

Preferably,
said herein mentioned first immunogenic fragment is an immunogenic fragment of a genotype P[7] rotavirus VP8 protein (X7), and/or
said herein mentioned second immunogenic fragment is an immunogenic fragment of a genotype P[13] rotavirus VP8 protein (X13), and/or
said herein mentioned third immunogenic fragment is an immunogenic fragment of a genotype P[6] rotavirus VP8 protein (X6), and/or
said herein mentioned fourth immunogenic fragment is an immunogenic fragment of a rotavirus C VP8 protein (XC).

Most preferably, said first immunogenic fragment is an immunogenic fragment of a genotype P[7] rotavirus VP8 protein (X7), and
said second immunogenic fragment is an immunogenic fragment of a genotype P[13] rotavirus VP8 protein (X13), and
said third immunogenic fragment is an immunogenic fragment of a genotype P[6] rotavirus VP8 protein (X6),
and optionally said fourth immunogenic fragment is an immunogenic fragment of a rotavirus C VP8 protein (XC).

Preferably,
said first immunogenic fragment consists of an amino acid sequence having at least 90%, preferably at least 95%, more preferably at least 98% or still more preferably at least 99% sequence identity with the sequence of SEQ ID NO:3, and
said second immunogenic fragment consists of an amino acid sequence having at least 90%, preferably at least 95%, more preferably at least 98% or still more preferably at least 99% sequence identity with the sequence of SEQ ID NO:5, and
said third immunogenic fragment consists of an amino acid sequence having at least 90%, preferably at least 95%, more preferably at least 98% or still more preferably at least 99% sequence identity with the sequence of SEQ ID NO:4, and
optionally said fourth immunogenic fragment consists of an amino acid sequence having at least 90%, preferably at least 95%, more preferably at least 98% or still more preferably at least 99% sequence identity with the sequence of SEQ ID NO:6.

In another preferred aspect,
said first polypeptide, as mentioned herein, is selected from the group consisting of R7, R13, R6 and RC, and/or
said second polypeptide, as mentioned herein, is selected from the group consisting R7, R13, R6 and RC, and/or
said third polypeptide is selected from the group consisting of R7, R13, R6 and RC, and/or
said fourth polypeptides is selected from the group consisting of R7, R13, R6 and RC;
wherein
R7 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12,
R13 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14,
R6 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:13, and
RC is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:15,
   in particular provided that all of said second to fourth polypeptides are different from said first polypeptide and different from each other.

Preferably, the at least one immunogenic substance, different from said first polypeptide, comprises or consists of a second polypeptide of a rotavirus VP8 protein, and
each of said first and second polypeptide is individually selected from the group consisting of R7, R13, R6 and RC,
provided that when said first polypeptide is R7, then said second polypeptide is selected from the group consisting of R13, R6 and RC,
provided that when said first polypeptide is R6, then said second polypeptide is selected from the group consisting of R7, R13 and RC,
provided that when said first polypeptide is R13, then said second polypeptide is selected from the group consisting of R7, R6 and RC,
and provided that when said first polypeptide is RC, then said second polypeptide is selected from the group consisting of R7, R13 and R6.

Particularly, the at least one immunogenic substance, different from said first polypeptide, comprises or consists of
a second polypeptide, and
a third polypeptide,
and each of said first to third polypeptides is individually selected from the group consisting of R7, R13, R6 and RC,
provided that when said first polypeptide is R7, then said second polypeptide is R13 and said third polypeptide is selected from the group consisting of R6 and RC,
provided that when said first polypeptide is R6, then said second polypeptide is R7 and said third polypeptide is selected from the group consisting of R13 and RC,
provided that when said first polypeptide is R13, then said second polypeptide is R7 and said third polypeptide is selected from the group consisting of R6 and RC,
and provided that when said first polypeptide is RC, then said second polypeptide is R7 and said third polypeptide is selected from the group consisting of R13 and R6.

More particularly, the at least one immunogenic substance, different from said polypeptide, comprises or consists of
a second polypeptide, and
a third polypeptide, and
a fourth polypeptide,
wherein each of said first to fourth polypeptides is individually selected from the group consisting of R7, R6, R13 and RC,
provided that when said first polypeptide is R7, then said second polypeptide is R13, said third polypeptide is R6 and said fourth polypeptide is RC,
provided that when said first polypeptide is R6, then said second polypeptide is R7, said third polypeptide is R13 and said fourth polypeptide is RC,
provided that when said first polypeptide is R13, then said second polypeptide is R7, said third polypeptide is R6 and said fourth polypeptide is RC,
and provided that when said first polypeptide is RC, then said second polypeptide is R7, said third polypeptide is R13 and said fourth polypeptide is R6.

According to a preferred aspect,
said herein mentioned first polypeptide is a protein comprising or consisting of an amino acid sequence having at least 70%, preferably at least 80%, more preferably at least 90%, still more preferably at least 95% or in particular 100% sequence identity with the sequence of SEQ ID NO:12, and
said herein mentioned second polypeptide is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, SEQ ID NO:13 and SEQ ID NO:15, and
preferably said herein mentioned third polypeptide is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, SEQ ID NO:13 and SEQ ID NO:15,
and optionally said herein mentioned fourth polypeptide is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, SEQ ID NO:13 and SEQ ID NO:15.

Particularly, an immunogenic composition of the present invention is preferred which comprises
(i) a protein comprising or consisting of an amino acid sequence having at least 70%, preferably at least 80%, more preferably at least 90%, still more preferably at least 95% or in particular 100% sequence identity with the sequence of SEQ ID NO:12, and
(ii) a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, SEQ ID NO:13 and SEQ ID NO:15.

Most particularly, an immunogenic composition of the present invention is preferred which comprises
(i) a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12, and
(ii) a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, and
   a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:13,
   and optionally a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:15.

The immunogenic composition of the present invention preferably comprises each of component (i) and of the substance(s) of component (ii) in a concentration of at least 100 nM, preferably of at least 250 nM, more preferably of at least 500 nM, and most preferably of at least 1 μM.

According to another preferred aspect, the immunogenic composition of the present invention contains each of component (i) and of the substance(s) of component (ii) in a concentration of 100 nM to 50 μM, preferably of 250 nM to 25 μM, and most preferably of 1-10 PM.

In particular 1 mL or, as the case may be, 2 mL of the immunogenic composition of the present invention are administered to a subject. Thus, a dose of the immunogenic composition of the present invention to be administered to a subject preferably has the volume of 1 mL or 2 mL.

Preferably one dose or two doses of the immunogenic composition are administered to a subject.

The immunogenic composition of the present invention is, preferably, administered systemically or topically. Suitable routes of administration conventionally used are parenteral or oral administration, such as intramuscular, intradermal, intravenous, intraperitoneal, subcutaneous, intranasal, as well as inhalation. However, depending on the nature and mode of action of a compound, the immunogenic composition may be administered by other routes as well. Most preferred is that the immunogenic composition is administered intramuscularly. The immunogenic composition of the present invention preferably further comprises a pharmaceutical- or veterinary-acceptable carrier or excipient.

As used herein, “pharmaceutical- or veterinary-acceptable carrier” includes any and all solvents, dispersion media, coatings, stabilizing agents, diluents, preservatives, antibacterial and antifungal agents, isotonic agents, adsorption delaying agents, and the like. In some preferred embodiments, and especially those that include lyophilized immunogenic compositions, stabilizing agents for use in the present invention include stabilizers for lyophilization or freeze-drying.

In some embodiments, the immunogenic composition of the present invention contains an adjuvant.

“Adjuvant” as used herein, can include aluminum hydroxide and aluminum phosphate, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopeia type); isoprenoid oil such as squalane or squalene; oil resulting from the oligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.), JohnWiley and Sons, NY, pp 51-94 (1995) and Todd et al., Vaccine 15:564-570 (1997). An exemplary adjuvant is the SPT emulsion described on page 147 of “Vaccine Design, The Subunit and Adjuvant Approach” edited by M. Powell and M. Newman, Plenum Press, 1995, or the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name CARBOPOL®; (BF Goodrich, Ohio, USA) are particularly appropriate. They are cross-linked with an allyl sucrose or with allyl pentaerythritol. Among then, there may be mentioned Carbopol 974P, 934P and 971P. Most preferred is the use of CARBOPOL® 971P. Among the copolymers of maleic anhydride and alkenyl derivative, are the copolymers EMA (Monsanto), which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants, from which the adjuvant may be chosen, include, but are not limited to, the RIBI adjuvant system (Ribi Inc.), Block co-polymer (CytRx, Atlanta GA), SAF-M (Chiron, Emeryville CA), monophosphoryl lipid A, Avridine lipid-amine adjuvant, heat-labile enterotoxin from E. coli (recombinant or otherwise), cholera toxin, IMS 1314 or muramyl dipeptide, or naturally occurring or recombinant cytokines or analogs thereof or stimulants of endogenous cytokine release, among many others.

It is expected that an adjuvant can be added in an amount of about 100 μg to about 10 mg per dose, preferably in an amount of about 100 μg to about 10 mg per dose, more preferably in an amount of about 500 μg to about 5 mg per dose, even more preferably in an amount of about 750 μg to about 2.5 mg per dose, and most preferably in an amount of about 1 mg per dose. Alternatively, the adjuvant may be at a concentration of about 0.01 to 50%, preferably at a concentration of about 2% to 30%, more preferably at a concentration of about 5% to 25%, still more preferably at a concentration of about 7% to 22%, and most preferably at a concentration of 10% to 20% by volume of the final product.

“Diluents” can include water, saline, dextrose, ethanol, glycerol, and the like. Isotonic agents can include sodium chloride, dextrose, mannitol, sorbitol, and lactose, among others. Stabilizers include albumin and alkali salts of ethylenediaminetetraacetic acid, among others.

According to a particular preferred aspect, the invention also provides an immunogenic composition, in particular the immunogenic composition of the present invention, wherein the immunogenic composition comprises or consists of
component (i),
component (ii),
a pharmaceutical- or veterinary-acceptable carrier or excipient,
and optionally an adjuvant.

The adjuvant in the context of the present invention is preferably selected from the group consisting of an emulsified oil-in-water adjuvant and a carbomer.

The term “immunogenic composition” refers to a composition that comprises at least one antigen, which elicits an immunological response in the host to which the immunogenic composition is administered. Such immunological response can be a cellular and/or antibody-mediated immune response to the immunogenic composition according to the invention. The host is also described as “subject”. Preferably, any of the hosts or subjects described or mentioned herein is an animal.

The term “animal”, as used herein, in particular relates to a mammal, preferably to swine, more preferably to a pig, most preferably to a piglet.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the immunogenic composition of the present invention. Preferably, the host will display either a protective immunological response or a therapeutic response.

A “protective immunological response” will be demonstrated by either a reduction or lack of one or more clinical signs normally displayed by an infected host, a quicker recovery time and/or a lowered duration of infectivity or lowered pathogen titer in the tissues or body fluids or excretions of the infected host.

The “pathogen” or “particular pathogen”, as mentioned herein, in particular relates to the rotavirus from which the immunogenic fragment of a rotavirus VP8 protein is derived. For example, the pathogen, as mentioned herein, is a rotavirus A or a rotavirus C.

In case where the host displays a protective immunological response such that resistance to new infection will be enhanced and/or the clinical severity of the disease reduced, the immunogenic composition is described as a “vaccine”.

An “antigen” as described herein refers to, but is not limited to, components which elicit an immunological response in a host to an immunogenic composition or vaccine of interest comprising such antigen or an immunologically active component thereof. In particular, the term “antigen” as used herein refers to a protein or protein domain, which, if administered to a host, can elicit an immunological response in the host.

The term “treatment and/or prophylaxis” refers to the lessening of the incidence of the particular pathogen infection in a herd or the reduction in the severity of one or more clinical signs caused by or associated with the particular pathogen infection. Thus, the term “treatment and/or prophylaxis” also refers to the reduction of the number of animals in a herd that become infected with the particular pathogen (=lessening of the incidence of the particular pathogen infection) or to the reduction of the severity of one or more clinical signs normally associated with or caused by an infection with the pathogen in a group of animals which animals have received an effective amount of the immunogenic composition as provided herein in comparison to a group of animals which animals have not received such immunogenic composition.

The “treatment and/or prophylaxis” generally involves the administration of an effective amount of the immunogenic composition of the present invention to a subject or herd of subjects in need of or that could benefit from such a treatment/prophylaxis. The term “treatment” refers to the administration of the effective amount of the immunogenic composition once the subject or at least some animals of the herd is/are already infected with such pathogen and wherein such animals already show some clinical signs caused by or associated with such pathogen infection. The term “prophylaxis” refers to the administration to a subject prior to any infection of such subject with a pathogen or at least where such animal or all of the animals in a group of animals do not show one or more clinical signs caused by or associated with the infection by such pathogen.

The term “an effective amount” as used herein means, but is not limited to an amount of antigen, in particular of the polypeptide(s) of the present disclosure, that elicits or is able to elicit an immune response in a subject. Such effective amount is able to lessen the incidence of the particular pathogen infection in a herd or to reduce the severity of one or more clinical signs of the particular pathogen infection. Preferably, one or more clinical signs are lessened in incidence or severity by at least 10%, more preferably by at least 20%, still more preferably by at least 30%, even more preferably by at least 40%, still more preferably by at least 50%, even more preferably by at least 60%, still more preferably by at least 70%, even more preferably by at least 80%, still more preferably by at least 90%, and most preferably by at least 95% in comparison to subjects that are either not treated or treated with an immunogenic composition that was available prior to the present invention but subsequently infected by the particular pathogen.

The term “clinical signs” as used herein refers to signs of infection of a subject from the particular pathogen. The clinical signs of infection depend on the pathogen selected. Examples for such clinical signs include but are not limited to diarrhea, vomiting, fever, abdominal pain, and dehydration.

Reducing the incidence of or reducing the severity of one or more clinical signs caused by or being associated with the particular pathogen infection in a subject can be reached by the administration of one or more doses of the immunogenic composition of the present invention to a subject.

The term “reducing fecal shedding” means, but is not limited to, the reduction of the number of RNA copies of a pathogenic virus, such as of a rotavirus, per mL of stool or the number of plaque forming colonies per deciliter of stool, is reduced in the stool of subjects receiving the composition of the present invention by at least 50% in comparison to subjects not receiving the composition and may become infected. More preferably, the fecal shedding level is reduced in subjects receiving the composition of the present invention by at least 90%, preferably by at least 99.9%, more preferably by at least 99.99%, and even more preferably by at least 99.999%.

The term “fecal shedding”, as used herein, is used according to its plain ordinary meaning in medicine and virology and refers to the production and release of virus from a cell of a subject into the environment from an infected subject via the stool of the subject.

The polypeptide of the present disclosure is preferably a recombinant protein, in particular a recombinant baculovirus expressed protein.

The term “recombinant protein”, as used herein, in particular refers to a protein which is produced by recombinant DNA techniques, wherein generally DNA encoding the expressed protein is inserted into a suitable expression vector which is in turn used to transform or, in the case of a virus vector, to infect a host cell to produce the heterologous protein. Thus, the term “recombinant protein”, as used herein, particularly refers to a protein molecule that is expressed from a recombinant DNA molecule. “Recombinant DNA molecule” as used herein refers to a DNA molecule that is comprised of segments of DNA joined together by means of molecular biological techniques. Suitable systems for production of recombinant proteins include but are not limited to insect cells (e.g., baculovirus), prokaryotic systems (e.g., Escherichia coli), fungi (e.g., Myceliophthora thermophile, Aspergillus oryzae, Ustilago maydis), yeast (e.g., Saccharomyces cerevisiae, Pichia pastoris), mammalian cells (e.g., Chinese hamster ovary, HEK293), plants (e.g., safflower), algae, avian cells, amphibian cells, fish cells, and cell-free systems (e.g., rabbit reticulocyte lysate).

According to another aspect, the present disclosure provides a polynucleotide comprising a sequence which encodes the polypeptide of the present disclosure, wherein said polynucleotide, which is also termed “the polynucleotide according to the present disclosure” hereinafter, is preferably an isolated polynucleotide.

Preferably, the polynucleotide according to the present disclosure comprises a nucleotide sequence having at least 70%, preferably at least 80%, more preferably at least 90%, still more preferably at least 95% or in particular 100% sequence identity with a sequence selected from the group consisting of SEQ ID NO:17, SEQ ID NO:18, SEQ ID NO:19, SEQ ID NO:20, and SEQ ID NO:21.

Production of the polynucleotides described herein is within the skill in the art and can be carried out according to recombinant techniques described, among other places, in Sam brook et al., 2001, Molecular Cloning, A Laboratory Manual, Cold Spring Harbor Laboratory Press, Cold Spring Harbor, NY; Amusable, et al., 2003, Current Protocols In Molecular Biology, Greene Publishing Associates & Wiley Interscience, NY; Innis et al. (eds), 1995, PCR Strategies, Academic Press, Inc., San Diego; and Erlich (ed), 1994, PCR Technology, Oxford University Press, New York, all of which are incorporated herein by reference.

In still a further aspect, the present disclosure provides a vector containing a polynucleotide which encodes the polypeptide of the present disclosure.

“Vector” as well as “vector containing a polynucleotide which encodes the polypeptide of the present disclosure”, for purposes of the present invention, refers to a suitable expression vector, preferably a baculovirus expression vector, which is in turn used to transfect, or in case of a baculovirus expression vector to infect, a host cell to produce the protein or polypeptide encoded by the DNA. Vectors and methods for making and/or using vectors (or recombinants) for expression can be made or done by or analogous to the methods disclosed in: U.S. Pat. Nos. 4,603,112, 4,769,330, 5,174,993, 5,505,941, 5,338,683, 5,494,807, 4,722,848, 5,942,235, 5,364,773, 5,762,938, 5,770,212, 5,942,235, 382,425, PCT publications WO 94/16716, WO 96/39491, WO 95/30018; Paoletti, “Applications of pox virus vectors to vaccination: An update,” PNAS USA 93: 11349-11353, October 1996; Moss, “Genetically engineered poxviruses for recombinant gene expression, vaccination, and safety,” PNAS USA 93: 11341-11348, October 1996; Smith et al., U.S. Pat. No. 4,745,051 (recombinant baculovirus); Richardson, C. D. (Editor), Methods in Molecular Biology 39, “Baculovirus Expression Protocols” (1995 Humana Press Inc.); Smith et al., “Production of Human Beta Interferon in Insect Cells Infected with a Baculovirus Expression Vector”, Molecular and Cellular Biology, December, 1983, Vol. 3, No. 12, p. 2156-2165; Pennock et al., “Strong and Regulated Expression of Escherichia coli B-Galactosidase in Infect Cells with a Baculovirus vector,” Molecular and Cellular Biology March 1984, Vol. 4, No. 3, p. 406; EPA0 370 573; U.S. application No. 920,197, filed Oct. 16, 1986; EP Patent publication No. 265785; U.S. Pat. No. 4,769,331 (recombinant herpesvirus); Roizman, “The function of herpes simplex virus genes: A primer for genetic engineering of novel vectors,” PNAS USA 93:11307-11312, October 1996; Andreansky et al., “The application of genetically engineered herpes simplex viruses to the treatment of experimental brain tumors,” PNAS USA 93: 11313-11318, October 1996; Robertson et al., “Epstein-Barr virus vectors for gene delivery to B lymphocytes”, PNAS USA 93: 11334-11340, October 1996; Frolov et al., “Alphavirus-based expression vectors: Strategies and applications,” PNAS USA 93:11371-11377, October 1996; Kitson et al., J. Virol. 65, 3068-3075, 1991; U.S. Pat. Nos. 5,591,439, 5,552,143; WO 98/00166; allowed U.S. application Ser. Nos. 08/675,556, and 08/675,566 both filed Jul. 3, 1996 (recombinant adenovirus); Grunhaus et al., 1992, “Adenovirus as cloning vectors,” Seminars in Virology (Vol. 3) p. 237-52, 1993; Ballay et al. EMBO Journal, vol. 4, p. 3861-65, Graham, Tibtech 8, 85-87, April, 1990; Prevec et al., J. Gen Virol. 70, 42434; PCT WO 91/11525; Felgner et al. (1994), J. Biol. Chem. 269, 2550-2561, Science, 259: 1745-49, 1993; and McClements et al., “Immunization with DNA vaccines encoding glycoprotein D or glycoprotein B, alone or in combination, induces protective immunity in animal models of herpes simplex virus-2 disease”, PNAS USA 93: 11414-11420, October 1996; and U.S. Pat. Nos. 5,591,639, 5,589,466, and 5,580,859, as well as WO 90/11092, WO93/19183, WO94/21797, WO95/11307, WO95/20660; Tang et al., Nature, and Furth et al., Analytical Biochemistry, relating to DNA expression vectors, inter alia. See also WO 98/33510; Ju et al., Diabetologia, 41: 736-739, 1998 (lentiviral expression system); Sanford et al., U.S. Pat. No. 4,945,050; Fischbach et al. (Intracel); WO 90/01543; Robinson et al., Seminars in Immunology vol. 9, pp. 271-283 (1997), (DNA vector systems); Szoka et al., U.S. Pat. No. 4,394,448 (method of inserting DNA into living cells); McCormick et al., U.S. Pat. No. 5,677,178 (use of cytopathic viruses); and U.S. Pat. No. 5,928,913 (vectors for gene delivery); as well as other documents cited herein.

Preferred viral vectors include baculovirus such as BaculoGold (BD Biosciences Pharmingen, San Diego, CA), in particular provided that the production cells are insect cells. Although the baculovirus expression system is preferred, it is understood by those of skill in the art that other expression systems, including those described above, will work for purposes of the present invention, namely the expression of recombinant protein.

Thus, the disclosure also provides a baculovirus containing a polynucleotide comprising a sequence which encodes the polypeptide of the present disclosure. Said baculovirus, which is also termed “the baculovirus according to the present disclosure” hereinafter, is preferably an isolated baculovirus.

Furthermore, the disclosure thus also provides a plasmid, preferably an expression vector, which comprises a polynucleotide comprising a sequence which encodes the polypeptide of the present disclosure. Said plasmid, which is also termed “the plasmid according to the present disclosure” hereinafter, is in particular an isolated plasmid.

The disclosure also provides a cell infected by and/or containing a baculovirus which comprises a polynucleotide comprising a sequence which encodes the polypeptide of the present disclosure, or a plasmid, preferably an expression vector, which comprises a polynucleotide comprising a sequence which encodes the polypeptide of the present disclosure. Said cell, which is also termed “the cell according to the present disclosure” hereinafter, is preferably an isolated cell.

The term “isolated”, when used in the context of an isolated cell, is a cell that, by the hand of man, exists apart from its native environment and is therefore not a product of nature.

In still another aspect, the invention also relates to the use of the immunogenic composition of the present invention for the preparation of a medicament, preferably of a vaccine.

In this context, the invention also provides a method of producing the immunogenic composition of the present invention, wherein said method comprises the step of infecting a cell, preferably an insect cell, with the baculovirus according to the present invention.

Furthermore, the disclosure also provides a method of producing the polypeptide of the present disclosure, wherein said method comprises the step of transfecting a cell with the plasmid according to the present disclosure.

The polypeptide of the present disclosure is preferably expressed in high amounts sufficient for the stable self-assembly of virus-like particles, which may then be used for vaccination.

The term “vaccination” or “vaccinating” as used herein means, but is not limited to, a process which includes the administration of an antigen, such as an antigen included in an immunogenic composition, to a subject, wherein said antigen, for instance the polypeptide of the present disclosure, when administered to said subject, elicits or is able to elicit, a protective immunological response in said subject.

The present invention also provides the immunogenic composition of the present invention for use as a medicament, preferably as a vaccine.

In particular, the immunogenic composition of the present invention is provided for use in a method of reducing or preventing one or more clinical signs or disease caused by a rotavirus infection, wherein the rotavirus is preferably a rotavirus of the group having a genome encoding the immunogenic fragment of a rotavirus VP8 protein. The immunogenic composition of the present invention is in particular provided for use in a method of reducing or preventing the fecal shedding caused by a rotavirus infection, wherein the virus is preferably a rotavirus of the group having a genome encoding the immunogenic fragment of a rotavirus VP8 protein. Thus, in one particular example, if the immunogenic fragment of a rotavirus VP8 protein, as mentioned herein, is encoded by the genome of a rotavirus A, then the immunogenic composition of the present invention is for use in a method of reducing or preventing one or more clinical signs, mortality, fecal shedding or disease caused by an infection with rotavirus A.

More particular, the immunogenic composition of the present invention is provided for use in a method of reducing or preventing one or more clinical signs, mortality or fecal shedding caused by a rotavirus infection in a subject or for use in a method of treating or preventing an infection with rotavirus in a subject.

A rotavirus infection, as mentioned herein, in particular refers to an infection with a rotavirus A or rotavirus C.

Furthermore, the immunogenic composition of the present invention is provided for use in a method for inducing an immune response against rotavirus in a subject.

The subject, as mentioned herein, is preferably a mammal, such as a swine or a bovine, or a bird, such as a chicken. In particular, the subject is a pig, and wherein the pig is preferably a piglet or a sow, such as a pregnant sow. Most preferably, in the context of inducing an immune response against rotavirus in a subject, said subject is a pregnant sow. In the context of reducing or preventing one or more clinical signs, mortality or fecal shedding caused by a rotavirus infection in a subject, or treating or preventing an infection with rotavirus in a subject, said subject is most preferably a piglet.

According to one preferred aspect, the immunogenic composition of the present invention is for use in a method of reducing or preventing one or more clinical signs, mortality or fecal shedding caused by a rotavirus infection in a piglet, wherein the piglet is to be suckled by a sow to which the immunogenic composition has been administered. Said sow to which the immunogenic composition has been administered is preferably a sow to which the immunogenic composition has been administered while said sow has been pregnant, in particular with said piglet.

Furthermore, the present invention relates to a method for the treatment or prevention of a rotavirus infection, the reduction, prevention or treatment of one or more clinical signs, mortality or fecal shedding caused by a rotavirus infection, or the prevention or treatment of a disease caused by a rotavirus infection, comprising administering the immunogenic composition of the present invention to a subject.

Also, a method for inducing the production of antibodies specific for rotavirus in a preferably pregnant sow is provided, wherein said method comprises administering the immunogenic composition of the present invention to said sow.

Furthermore, the present invention provides a method of reducing or preventing one or more clinical signs, mortality or fecal shedding caused by a rotavirus infection in a piglet, wherein said method comprises
administering the immunogenic composition of the present invention to a sow, and
allowing said piglet to be suckled by said sow,
   and wherein said sow is preferably a sow being pregnant, in particular with said pig.

Preferably, said two foregoing methods comprise the steps of
administering the immunogenic composition of the present invention to a sow being pregnant with said piglet,
allowing said sow to give birth to said piglet, and
allowing said piglet to be suckled by said sow.

Moreover, a method of reducing one or more clinical signs, mortality or fecal shedding caused by a rotavirus infection in a piglet is provided, wherein the piglet is to be suckled by a sow to which the immunogenic composition of the present invention has been administered.

The one or more clinical signs, as mentioned herein, are preferably selected from the group consisting of
diarrhea,
rotavirus colonization, in particular rotavirus colonization of the intestine,
lesions, in particular macroscopic lesions, and
decreased average daily weight gain.

According to one example, the one or more clinical signs mentioned herein are a rotavirus colonization of the intestine, in particular of the small intestine. According to another example, the one or more clinical signs mentioned herein are enteric lesions, in particular macroscopic enteric lesions.

According to another particular preferred aspect, the immunogenic composition of the present invention is for use in any of the above described methods, wherein
said rotavirus infection is an infection with genotype P[23] rotavirus and/or genotype P[7] rotavirus,
said infection with rotavirus is an infection with genotype P[23] rotavirus and/or genotype P[7] rotavirus,
said immune response against rotavirus is an immune response against genotype P[23] rotavirus and/or genotype P[7] rotavirus, or
said antibodies specific for rotavirus are antibodies specific for genotype P[23] rotavirus and/or genotype P[7] rotavirus,
   and wherein preferably said immunogenic composition of the present invention comprises any of the polypeptides of the present disclosure described herein comprising an immunogenic fragment of a genotype P[7] rotavirus VP8 protein, in particular consisting of an amino acid sequence having at least 90%, preferably at least 95%, more preferably at least 98% or still more preferably at least 99% sequence identity with the sequence of SEQ ID NO:3.

In one particular aspect, an “infection with genotype P[23] rotavirus and/or genotype P[7] rotavirus”, as mentioned herein, is an infection with genotype P[23] rotavirus.

In another preferred aspect, an “infection with genotype P[23] rotavirus and/or genotype P[7] rotavirus”, as mentioned herein, is an infection with genotype P[23] rotavirus and genotype P[7] rotavirus.

In one particular aspect, an “immune response against genotype P[23] rotavirus and/or genotype P[7] rotavirus”, as mentioned herein, is an immune response against genotype P[23] rotavirus.

In another preferred aspect, an “immune response against genotype P[23] rotavirus and/or genotype P[7] rotavirus”, as mentioned herein, is an immune response against genotype P[23] rotavirus and genotype P[7] rotavirus.

In one particular aspect, the “antibodies specific for genotype P[23] rotavirus and/or genotype P[7] rotavirus”, as mentioned herein, are antibodies specific for genotype P[23] rotavirus.

In another preferred aspect, the “antibodies specific for genotype P[23] rotavirus and/or genotype P[7] rotavirus”, as mentioned herein, comprise or are antibodies specific for genotype P[23] and antibodies specific for genotype P[7] rotavirus.

In a further aspect, the immunogenic composition of the present invention is administered for inducing the production of antibodies specific for rotavirus C, in an animal, preferably in a pregnant sow. Preferably in this further aspect, said immunogenic composition of the present invention comprises, respectively, any of the polypeptides of the present disclosure described herein comprising an immunogenic fragment of a rotavirus C VP8 protein, in particular consisting of an amino acid sequence having at least 90%, preferably at least 95%, more preferably at least 98% or still more preferably at least 99% sequence identity with the sequence of SEQ ID NO:15.

The invention further provides a method of producing the immunogenic composition of the present invention, wherein said method comprises transfecting a cell with the plasmid of the present invention.

Furthermore, a method of producing the immunogenic composition of the present invention is provided, wherein said method comprises infecting a cell, preferably an insect cell, with the baculovirus of the present disclosure.

Also, the present invention relates to a method of producing the immunogenic composition of the present invention, wherein the method comprises the steps of:
(a) permitting infection of susceptible cells in culture with a vector comprising a nucleic acid sequence encoding the polypeptide of the present disclosure, wherein said polypeptide is expressed by said vector;
(b) thereafter recovering said polypeptide, in particular in the supernatant of said cultured cell, wherein preferably cell debris is separated from said polypeptide via a separation step, preferably including a micro filtration through at least one filter, preferably two filters, wherein the at least one filter preferably has a pore size of about 1 to about 20 μm and/or about 0.1 μm to about 4 μm;
(c) inactivating the vector by adding binary ethylenimine (BEI) to the mixture of step (b);
(d) neutralizing the BEI by adding sodium thiosulfate to the mixture resulting from step (c); and
(e) concentrating the polypeptide in the mixture resulting from step (d) by removing a portion of the liquid from the mixture by a filtration step utilizing a filter with a filter membrane having a molecular weight cut off of between about 5 kDa and about 100 kDa, preferably between about 10 kDa and about 50 kDa;
   and wherein said method comprises the steps of:
(f) admixing the mixture remaining after step (e) with at least one immunogenic substance, different from said polypeptide;
(g) and optionally admixing the mixture remaining after step (f) with a further component selected from the group consisting of pharmaceutically acceptable carriers, adjuvants, diluents, excipients, and combinations thereof,
   or wherein said method comprises the steps of:
(f) admixing the mixture remaining after step (e) with a further component selected from the group consisting of pharmaceutically acceptable carriers, adjuvants, diluents, excipients, and combinations thereof, and
(g) admixing the mixture remaining after step (f) with at least one immunogenic substance, different from said polypeptide.

For said admixing with at least one immunogenic substance, different from said polypeptide, in particular any of the herein described
at least one immunogenic substance, different from said polypeptide,
or component (ii), respectively,
is used.

In step (a) of said method, said cells are preferably insect cells and said vector is preferably the baculovirus of the present disclosure.

In step (b) of said method, said polypeptide is most preferably recovered in the supernatant of said cultured cells, rather than from inside the cells.

Furthermore, the present invention provides the immunogenic composition of the present invention and the use of said immunogenic composition in any of the herein described methods, wherein said immunogenic composition is obtainable by the aforementioned method of producing the immunogenic composition of the present invention.

### EXAMPLES

The following examples are only intended to illustrate the present disclosure. They shall not limit the scope of the claims in any way.

### Example 1

### Design, Production and Testing of Fusion Proteins:

### Construct Design:

The rotavirus A VP4 sequence was originally obtained from a swine fecal sample which most closely matches GenBank sequence JX971567.1 and is classified as a P[7] genotype. VP4 amino acids 57-224 (SEQ ID NO:3), also named “AVP8” hereinafter, were used and correspond to the lectin-like domain of the VP8 protein but with an N-terminus extended by eight amino acid residues. The linker moiety is Gly-Gly-Ser (SEQ ID NO:9). The Swine IgG Fc sequence (SEQ ID NO:7) matches amino acids 242-470 of IgG heavy chain constant precursor (GenBank sequence BAM75568.1). An IDT Gblock encoding AVP8, the Gly-Gly-Ser linker, and Swine IgG Fc sequence, all codon optimized for insect cells, was received (SEQ ID NO:17), and named AVP8-IgG Fc herein. The protein (SEQ ID NO:12) encoded by AVP8-IgG Fc is also termed “AVP8-IgG Fc protein” herein.

### Cloning, Expression, and Purification:

AVP8-IgG Fc was TOPO cloned and subsequently inserted into baculovirus transfer plasmid pVL1393 using the BamHI and NotI restriction sites, then co-transfected into Sf9 cells with BaculoGold to generate recombinant baculoviruses. Production of AVP8-IgG Fc protein was done as follows: 1 L of Sf+ cells in a 3 L spinner flask was infected at 0.2MOI with spent media harvested 4 DPI, centrifuged 20 minutes at 15,000 g, and 0.2 μm filtered. 1 mL of MabSelect SuRE LX resin slurry (GE Healthcare, cat #17-5474-01) was added and incubated overnight at 4° C. with moderate stirring. Resin was recaptured by filtrations, washed 4×10 mL Gentle Binding Buffer (Pierce, cat #21012), and eluted in 7×5 mL volumes of Gentle Elution Buffer (Pierce, cat #21027). Fractions were combined and dialyzed at 4° C. against 3.5 L TBS with one buffer change. A BCA assay (Thermo Scientific, cat #23227) was done to determine concentration (80 μg/mL).

### Serology Study:

Protein-A purified AVP8-IgG Fc protein was formulated with Emulsigen D with 87.5% antigen and 12.5% adjuvant. Pigs of approximately seven weeks of age received a 2 mL dose by IM on the side of the neck, with a boost 21 days later. Sera samples were collected weekly for seven weeks. Serum from pigs vaccinated with AVP8-IgG Fc protein were assessed by ELISA (FIG. 1), as described below (“Protocol for ELISA”), and virus neutralization assay (FIG. 2), as described below (“Protocol for virus neutralization assay”). In comparison to a non-relevant vaccine control, the IgG ELISA results from pigs vaccinated with AVP8-IgG Fc protein showed an increase in SP ratio peaking at day 14 and rising again after the boost on day 21. Virus neutralization titers similarly showed an increase on days 7 and 14, followed by a second peak on day 28 following the boost on day 21.

### Protocol for ELISA

For IgA ELISA, medium protein binding 96-well ELISA plates were coated with whole rotavirus antigen diluted in 1×PBS 1:16. Plates were incubated at a temperature of 4° C. overnight. Following incubation, plates were washed using 1×PBST and then blocked with Casein blocking solution for 1 hour @ 37° C. Following washing, 100 μL of primary antibodies diluted to a final dilution of 1:40 in blocking buffer were added to plates and incubated for 1 hour @37° C. Following washing, wells were coated with 100 μl of a 1:3200 dilution of horse-radish peroxidase (HRP)-conjugated-goat-anti-swine-IgA and incubated for one hour at 37° C. Following washing, the plate was developed with 3,5,3′,5′-tetramethylbenzidine for 15 minutes at room temperature and the reaction was stopped with 1 N HCl before optical density (OD) measurement at 450 nm. Samples, including a positive and negative control, were run in duplicate wells and results are reported as the average of (sample−negative control)-to-(positive−negative control) ratio (S−N)/(P−N).

For IgG ELISA, medium protein binding 96-well ELISA plates were coated with whole rotavirus antigen diluted in 1×PBS 1:8. Plates were incubated at a temperature of 4° C. overnight. Following incubation, plates were washed using 1×PBST and then blocked with Blotting grade blocking solution for 1 hour @ 37° C. Following washing, 100 μL of primary antibodies diluted to a final dilution of 1:625 in blocking buffer were added to plates and incubated for 1 hour @37° C. Following washing, wells were coated with 100 μl of a 1:8000 dilution of horse-radish peroxidase (HRP)-conjugated-goat-anti-swine-IgG and incubated for one hour at 37° C. Following washing, the plate was developed with 3,5,3′,5′-tetramethylbenzidine for 10 minutes at room temperature and the reaction was stopped with 1 N HCl before optical density (OD) measurement at 450 nm. Samples, including a positive and negative control, were run in duplicate wells and results are reported as the average of (sample−negative control)-to-(positive−negative control) ratio (S−N)/(P−N).

### Protocol for Virus Neutralization Assay

All serum and milk samples were heat inactivated at 56° C. for 30 minutes. Samples were serially diluted from 1:40 through 1:2,560 in rotavirus growth media (MEM+2.5% HEPES+0.3% Tryptose phosphate broth+0.02% yeast+10 μg/mL trypsin). Rotavirus A isolate (titer 7.0 log TCID₅₀/mL) was diluted 1:25,000 into rotavirus growth media. A total of 200 μl of the diluted serum was added to 200 μl of the diluted virus; the mixture was incubated at 37° C.±5% CO₂ for one hour. Growth media was aseptically removed from three-four day old 96-well plates planted with MA104 cells. Following incubation, 200 μl of the virus-serum mixture was transferred to the cell culture plates. Cells were incubated at 37° C.±5% CO₂ for 72 hours. The stock and diluted virus were titrated on the day of use to confirm the dilution used in the assay. Following incubation, the supernatant was discarded and plates were washed once with 200 μL/well 1×PBS. For fixation, 100 μL/well of 50%/50% acetone/methanol was added. Plates were incubated at room temperature for 15 minutes, air-dried, then rehydrated with 100 μL/well 1×PBS. The primary antibody (Rabbit anti-Rotavirus A polyclonal serum, internally generated) was diluted 1:1000 in 1×PBS. 100 μL/well of the diluted primary antibody was added and plates were incubated at 37° C.±5% CO₂ for one hour. Following incubation, plates were washed twice with 100 μL/well of 1×PBS. The secondary antibody (Jackson ImmunoResearch FITC labeled goat-anti-rabbit IgG cat #111-095-003) was diluted 1:100 in 1×PBS. 100 μL/well of the diluted secondary antibody was added and plates were incubated at 37° C.±5% CO₂ for one hour. Following incubation, plates were washed twice with 100 μL/well of 1×PBS. Plates were read for the presence of fluorescence using an ultraviolet microscope. The assay was considered valid if the titer (generated using the Reed-Muench method) of the diluted virus was found to be 2.8±0.5 log TCID₅₀/mL. In addition, known positive and negative samples were included in each assay as a control. Serum titers were reported as the highest dilution in which no staining was observed.

### Example 2

### Challenge Studies:

The primary purpose of this study was to evaluate whether administration of a prototype vaccine, also termed “IgG:AVP8” herein, including AVP8-IgG Fc protein (SEQ ID NO:12) and a non-relevant control vaccine, termed “Placebo” herein, to conventional sows conferred passive protection to pigs against a virulent rotavirus A challenge. Furthermore, for comparison, a commercially available MLV rotavirus vaccine (ProSystem® Rota, Merck Animal Health), also termed “commercial product” or “Commercial vaccine” herein, was used in the study. The prototype vaccine, was produced similarly to the production described above in Example 1, but with different volumes used for the infection and a longer incubation period, as described below in the section “Production of IgG:AVP8”. The commercial product was used according to the label instructions (dosage and directions, as well as the recommended Method for oral vaccination of swine) provided by the manufacturer for the vaccine ProSystem® TGE/Rota.

A total of 16 sows were included in the study. Sows were randomized into three treatment groups and one strict control group as described in Table 1 below. Sows in T02 and T04 were comingled between three rooms. Sows in T06 and T07 were housed in two separate rooms. All sows were vaccinated with the appropriate material by the appropriate route as listed in Table 1. Sows in T07 remained non-vaccinated (strict control). Serum was collected from the sows periodically throughout the vaccination period and assayed for evidence of seroconversion. Fecal samples were collected prior to farrowing and screened by RT-qPCR to confirm dams were not actively shedding rotavirus prior to farrowing. General health observations were recorded on each sow daily. Farrowing was allowed to occur naturally until the sow reached gestation day 114. After this time, farrowing was induced. Piglets were enrolled into the trial at the time of farrowing. Only piglets which were healthy at birth were tagged, processed according to facility standard operating procedures, and included in the trial. When pigs were zero to five days of age, they were bled, a fecal swab was collected, and pigs were challenged (excluding T07). At the time of challenge, pigs were administered an intragastric, 5 mL dose of sodium bicarbonate, then an intragastric, 5 mL dose of the challenge material. Throughout the challenge period, all animals were monitored daily for the presence of enteric disease (diarrhea, and behavior changes). Fecal samples were collected periodically throughout the challenge period. At two days post challenge (DPC 2), approximately one-third of the pigs from each litter were euthanized. Following euthanasia, a necropsy was performed and pigs were evaluated for macroscopic lesions. Intestinal sections were collected for microscopic and immunohistological evaluation. An intestinal swab was collected for RT-qPCR evaluation. At DPC 21, all remaining pigs were weighed, bled, and a fecal swab was collected. Following sample collection, pigs were euthanized. Pigs were evaluated for macroscopic lesions and an intestinal swab was collected.

TABLE 1
Study Design

| | | | | | | Piglet | |
|---|---|---|---|---|---|---|---|
| | | | | Sow vaccination | | challenge | |
| | N | N | | (6 & 2 wks pre-farrow) | | (DPC0; 0-5 | |
| Group | (sows) | (piglets) | Room | Description | Route/dose* | days of age) | Necropsy |
| T02 | 6 | 57 | Comingled | Placebo | 2 mL IM + | Tissue | 1/3 pigs |
| | | | between | | 2 mL IN | homogenate | necropsied |
| T04 | 5 | 46 | rooms | IgG:AVP8 | 2 mL IM | 1:2 dilution 1 | on DPC2; |
| | | | 115, 116, | | | mL dose | remaining |
| | | | & 117 | | | intragastrically | pigs on |
| T06 | 2 | 22 | 118 | Commercial | 2 mL oral at | | DPC21 |
| | | | | vaccine | 5 & 2 wks | | |
| | | | | | pre-farrow + | | |
| | | | | | 2 mL IM at 1 | | |
| | | | | | wk pre- | | |
| | | | | | farrow | | |
| T07 | 3 | 27 | 114 | Strict | Not | None | Not |
| | | | | control | applicable | | applicable |

*IM = intramuscular, IN = intranasal

Throughout the study, serum VN titers in sows from T07 (strict control) either remained constant or declined indicating lack of exposure and a valid study (virus neutralization was assessed as described above in Example 1 (“Protocol for virus neutralization assay”), results are shown in FIG. 3). During the vaccination phase, the highest median VN titers in serum were observed in sows vaccinated with the IgG:AVP8 (T04) prototype vaccine. In this group, one dose administered at six weeks pre-farrow resulted in four-fold or greater increased titer in 3/5 animals in T04 (IgG:AVP8) by D14. Prior to the time of pig challenge, 5/5 animals in T04 (IgG:AVP8) had a four-fold or greater increase in titer. Sows in the placebo group (T02) had no significant increase (<2-fold) in serum VN titer during the vaccination phase. Sows in T06 (Commercial vaccine), had no significant increase (<2-fold) in serum VN titer through D35. Prior to the time of pig challenge, both sows of T06 (Commercial vaccine) had a four-fold increase in titer. Following lateral exposure to challenge material, VN serum titers in sows in T02 (Placebo) and T06 (Commercial vaccine) increased. Conversely, VN serum titers in sows in T04 (IgG:AVP8) remained constant or decreased in 4/5 sows. In regards to colostrum and milk VN titers, in group T04 (IgG:AVP8), VN titers were highest at farrowing, decreased in the pre-challenge sample and further decreased in the post-challenge sample. In the placebo group (T02), VN titers were low at farrowing and pre-challenge but increased following lateral exposure to the challenge material.

The VN titers in pig serum pre-challenge were high (>1280) in the majority of pigs in T04 (IgG:AVP8) indicating passive transfer of immunity from sows to pigs. Conversely, the majority of titers in pigs in T02 (Placebo) and T06 (Commercial vaccine) were low (<1280).

Throughout the challenge phase, the highest numbers of mortalities were observed in T02 (Placebo) with 8/57 (14.0%) of pigs dying. Conversely, only 1/46 (2.2%) pigs died in T04 (IgG:AVP8), 1/22 (4.5%) pigs died in T06 (Commercial vaccine), and 1/27 (3.7%) pigs died in T07 (Strict control). No clinical signs of diarrhea were observed in pigs in T07 (strict control) throughout the study. Clinical signs of diarrhea in pigs in T02 (Placebo) began on days post challenge (DPC)1 or 2 and resolved in the majority of animals by DPC10. Overall, clinical signs of diarrhea were observed in 44/57 (77.2%) of animals in T02 (Placebo) at least once during the study. Of these 44 animals, diarrhea was considered severe in 29 (65.9%) of the animals. In contrast, clinical signs of diarrhea were reduced in pigs in T04 (IgG:AVP8). See Table 2 below for a summary of the clinical diarrhea results by group.

TABLE 2
Percentage of animals with abnormal diarrhea (ever) by group

| | | |
|---|---|---|
| Group | Ever abnormal* | Ever severe** |

| | | | | |
|---|---|---|---|---|
| T02- Placebo | 44/57 | (77.2%) | 29/44 | (65.9%) |
| T04- IgG:AVP8 | 15/46 | (32.6%) | 8/15 | (53.3%) |
| T06- Commercial vaccine | 13/22 | (59.1%) | 10/13 | (76.9%) |

| | | | |
|---|---|---|---|
| T07- Strict control | 0/27 | (0.0%) | Not applicable |

*Includes pigs with a score of 1 or 2 at least once during the study divided by the total number of pigs per group
**Includes pigs with a score of 2 at least once during the study divided by the total number of pigs that were ever abnormal

Prior to challenge, there was no detection of rotavirus A RNA by RT-qPCR indicating a valid study. In addition, there was no detection of rotavirus A RNA by RT-qPCR in sows or pigs from T07 (Strict controls) throughout the study. In pigs following challenge, shedding was most prevalent in T02 (Placebo). In the majority of pigs, shedding began on DPC1-3 and continued through DPC14. Of most interest was the reduction in shedding observed in T04 (IgG:AVP8) as compared to T02 (Placebo) and T06 (Commercial vaccine). Both the percentage of shedding and median amounts of RNA detected were reduced (see FIG. 4 for the group median log rotavirus A RNA genomic copies (gc)/mL in feces by study day); the testing was done as described below (“Protocol for Rota A qRT-PCR”).

A randomly selected subset of pigs from each group were euthanized and necropsied at DPC2. Pigs were evaluated for the presence of macroscopic enteric lesions (thin-walled, gas-distended small intestine, pure liquid content, etc), microscopic lesions (atrophic enteritis), and Rotavirus A specific staining by immunohistochemistry (IHC). Table 3 below presents the number of pigs with enteric lesions at the time of necropsy by group. The challenge was considered successful as 84.2% (16/19) of pigs in the placebo group (T02) had macroscopic lesions and of those 63.2% (12/19) had staining. Of most interest was the lack of Rotavirus A staining in animals in only 1/15 pigs in T04 (IgG:AVP8). In addition, in T04 (IgG:AVP8) there was a reduction in the percentage of pigs with macroscopic lesions in comparison to T02 (Placebo) and the commercial product (T06).

TABLE 3
Percentage of animals with enteric lesions and
IHC staining at the time of necropsy by group.

| | Enteric | IHC staining at DPC2** | | | |
|---|---|---|---|---|---|
| | lesions at | | No. | No. | No. |
| Group | DPC2* | % pos | Score 1 | Score 2 | Score 3 |
| T02- Placebo | 16/19 | 12/19 | 2/12 | 2/12 | 8/12 |
| | (84.2%) | (63.2%) | (16.7%) | (16.7%) | (66.6%) |
| T04- IgG:AVP8 | 4/15 | 1/15 | 0/1 | 0/1 | 1/1 |
| | (26.7%) | (6.7%) | (0.0%) | (0.0%) | (100.0%) |
| T06- Commercial | 4/8 | 4/8 | 3/4 | 1/4 | 0/4 |
| vaccine | (50.0%) | (50.0%) | (75.0%) | (25.0%) | (0.0%) |

| T07- Strict | Not | Not applicable^{§} |
|---|---|---|
| control | applicable^{§} | |

*Represents the number of pigs with enteric lesions at DPC2 divided by the total number of pigs necropsied at DPC2
**Where Score 1 = <10% of villi contain antigen, Score 2 = 10% to 50% of villi contain antigen, Score 3 = >50% of villi contain antigen
^{§}Not applicable as pigs from T07 were not necropsied

The average daily weight gain was calculated for surviving pigs (in kg) and is presented in Table 4 below. The highest numerical benefits in ADWG were observed in pigs from T04 (IgG:AVP8). The increase in ADWG following vaccination was significantly different in comparison to T02 (Placebo).

TABLE 4
Mean average daily weight gain in
kg (standard deviation) by group.

| | ADWG in kg |
|---|---|
| Group | Mean (Std. dev.) |
| T02- Placebo | 0.15 (0.13) |
| T04- IgG:AVP8 | 0.25 (0.08) |
| T06- Commercial vaccine | 0.22 (0.11) |
| T07- Strict control | 0.23 (0.07) |

In conclusion, vaccination of conventional sows at six- and two-weeks prefarrow with the IgG:AVP8 prototype vaccine (comprising the polypeptide of SEQ ID NO:12) lead to high neutralizing antibody titers in sow serum and colostrum. These neutralizing antibodies were passively transmitted to pigs following birth as evidenced by detection of high titers (>1280) in the serum of pigs from vaccinated sows. The presence of high neutralizing antibody titers in the pigs lead to clinical protection. Specifically, pigs born to vaccinated sows had reduced fecal shedding of rotavirus A RNA, reduced mortality, reduced clinical signs of diarrhea, reduced colonization of rotavirus A at DPC2, reduced macroscopic lesions at DPC2, and increased ADWG as compared to pigs born to placebo controls and the commercially available vaccine.

Protocol for Rota A qRT-PCR

In order to determine Rotavirus A RNA in the fecal samples the quantitative one-step RT-PCR kit (iTaq Universal One-Step RT-PCR kit; BioRad, cat no. 1725140) was used for the assay. See Table 5 below for primer and probe information.

TABLE 5
Primer (F/R) and probe (Pr1/Pr2) information

| Name | Sequence | Size | Position |
|---|---|---|---|
| RVA F | 5′-GCT AGG GAY AAA ATT GTT GAA GGT A-3′ (SEQ ID NO: 22) | 25 | 40..64 |
| RVA R | 5′-ATT GGC AAA TTT CCT ATT CCT CC-3′ (SEQ ID NO: 23) | 23 | 145..167 |
| RVA Pr1 | 5′-FAM-ATG AAT GGA AAT GAY TTT CAA AC-MGB-3′ (SEQ ID | 23 | 121..143 |
| | NO: 24) | | |
| RVA Pr2 | 5′-FAM-ATG AAT GGA AAT AAT TTT CAA AC-MGB-3′ (SEQ ID | 23 | 121..143 |
| | NO: 25) | | |

Real-time RT-PCR was carried out in a 20 μl reaction containing 5 μl of extracted total nucleic acid, 1 μl of each probe (5 μM), 1 μl of each primer (10 μM), 10 μl of 2× RT-PCR mix, 0.5 μl iScript reverse transcriptase and 0.5 μl of DEPC-treated water. The reaction took place using a CFX96 real-time PCR detection system (BioRad) under the following conditions: initial reverse transcription at 50° C. for 10 min, followed by initial denaturation at 95° C. for 3 min, 40 cycles of denaturation at 95° C. for 15 s and annealing and extension at 60° C. for 45 s. To generate relative quantitative data, serial dilutions of two Rotavirus A g-blocks were included in each run. Equal amounts of each of the g-blocks were included in the run using 5.0×10⁷ genomic copies/μL as the starting concentration. The optical data were analyzed using CFX Manager software. For each determination, the threshold lines were automatically calculated using the regression setting for cycle threshold (Ct) determination mode. Baseline subtraction was done automatically using the baseline subtracted mode. Curves with baseline end values of less than 10 were manually corrected.

### Production of IgG:AVP8

2 L of Sf+ (Spodoptera frugiperda) cells at an approximate concentration of 1×10⁶ cells/mL in a 5 L shaker flask were infected with 1.7 mL of a recombinant baculovirus stock containing the Rotavirus A VP8 core-swine IgG Fc fusion protein (BaculoGold (BG)/pVL1393-AVP8-IgG; 1.18×108 TCID50/mL). The shaker flask was incubated at 28° C.±2° C. with constant agitation at 90 rpm for five days. Cells and media were aseptically transferred to 3×1 L centrifuge bottles and cells were pelleted at 10,000 g for 20 minutes at 4° C. The resulting supernatant was passed through a 0.2 μm filter (Thermo Scientific, cat #567-0020) then incubated with 2.5 mL of MabSelect SuRe LX protein A resin (GE Healthcare cat #17-5474-01) overnight at 4° C. with moderate stirring. Resin was recovered by 0.2 μm filtration (Thermo Scientific, cat #567-0020) then washed with 12×10 mL volumes of Gentle Ag/Ab Binding Buffer (Thermo Scientific, cat #21012). AVP8-IgG was eluted from the resin using 7×10 mL volumes of Gentle Ag/Ab Elution Buffer (Thermo Scientific, cat #21027). AVP8-IgG was dialyzed against 3.5 L of 20 mM Tris pH 7.5, 150 mM NaCl with one buffer change. Residual baculovirus was inactivated with 5 mM BEI for 24 hours at 37° C. The resulting material was diluted to a target concentration of 70 μg/mL in 1×PBS (Gibco cat #10010-023). The diluted material was formulated with 12.5% Emulsigen D.

### Example 3

### Serology Study:

The primary purpose of this study was to evaluate whether administration of a prototype vaccine including AVP8-IgG Fc protein (SEQ ID NO:12)) and a control vaccine, termed “Placebo” herein, to conventional sows generated a serological response against rotavirus A. The prototype vaccine (either comprising Emulsigen D or Carbopol as an adjuvant, c.f. Tables 7 A and 7 B below), also termed “IgG-AVP8” herein, was produced similarly to the production described above in Examples 1 and 2, but with different volumes used for the infection and a longer incubation period, as described below in the section “Vaccine Production: IgG-AVP8”.

A total of 20 sows were included in the study. Sows were randomized into four treatment groups as described in Table 6 below. Sows were comingled throughout the study. All sows were vaccinated with the appropriate material intramuscularly on DO and D21 as listed in Table 4. Serum was collected from the sows periodically throughout the study and assayed for evidence of seroconversion by virus neutralization assay. General health observations were recorded on each sow daily. The study was terminated on D42.

TABLE 6
Study Design

| | | Material used for IM* | | |
|---|---|---|---|---|
| | | Vaccination | Blood collection | Study |
| Group | N | at D 0 and D 21 | dates | termination |
| T02 | 8 | IgG-AVP8/Emulsigen D | D 0, 7, 14, 21, | D 42 |
| T03 | 8 | IgG-AVP8/Carbopol | 28, 35, 42 | |
| T06 | 2 | Placebo/Emulsigen D | | |
| T07 | 2 | Placebo/Carbopol | | |

*IM = intramuscular

Throughout the study, serum VN titers in sows from T06 and T07 (placebo groups) either remained constant or declined indicating lack of exposure and a valid study (virus neutralization was assessed as described above in Example 1 (“Protocol for virus neutralization assay”) with the modification that increased dilutions were evaluated—1:40 through 1:40,960). During the vaccination phase, sows vaccinated with the IgG-AVP8/Emulsigen D (T02) and IgG-AVP8/Carbopol (T03) prototype vaccines had significant increases in titer (>4 fold). For both groups (T02 and T03), group mean titers were above 640 following one vaccination and remained above 640 throughout the study period. In contrast, sows in the placebo groups (T06 and T07) had no significant increase (<2-fold) in serum VN titer throughout the study.

In conclusion, vaccination of conventional sows at six- and two-weeks prefarrow with the IgG-AVP8 prototype vaccine (comprising the polypeptide of SEQ ID NO:12) lead to high neutralizing antibody titers in sow serum.

### Vaccine Production: IgG-AVP8

8 L of Sf+ cells at 1.00×10\hat{ }6 cells/mL in a 10 L Sartorius Biostat B glass-jacketed vessel were infected with 15 mL of BG/pVL1393-AVP8-IgG, 1.19×10\hat{ }8 TCID50/mL, for an MOI of 0.22. Bioreactor was run at 27° C. with 100 rpm agitation and oxygen sparged at 0.3 slpm. Vessel was harvested at 6 DPI, centrifuged at 10,000 g and 4° C. for 20 minutes, and supernatant 0.8/0.2 μm filtered (GE Healthcare, cat #6715-7582). 2750 mL of clarified supernatant was inactivated with 5 mM BEI for five days at 27° C. Following neutralization of residual BEI with sodium thiosulfate, 2750 mL was concentrated approximately 12× using a 10 kDa hollow fiber filter (GE, cat #UFP-10-C-4MA) to 225 mL. Concentration was determined to be 255 μg/mL.

TABLE 7 A
Vaccine formulation

| Component | Purpose | Volume | | Concentration |
|---|---|---|---|---|
| AVP8-IgG protein | Antigen | 16.5 | mL | 27.5% |
| PBS | Diluent | 31.5 | mL | 52.5% |
| Carbopol | Adjuvant | 12 | mL | 20% |

TABLE 7 B
Vaccine formulation

| Component | Purpose | Volume | | Concentration |
|---|---|---|---|---|
| AVP8-IgG protein | Antigen | 16.5 | mL | 27.5% |
| PBS | Diluent | 36 | mL | 60% |
| Emulsigen D | Adjuvant | 7.5 | mL | 12.5% |

### Example 4

The primary purpose of this study was to evaluate whether animals vaccinated with IgG-AVP8 (including AVP8-IgG Fc protein (SEQ ID NO:12)) would be able to cross neutralize various Rotavirus A serotypes/genotypes of various G and P types other than P[7], from which the AVP8-IgG Fc protein was designed. This would indicate the ability of the AVP8-IgG Fc protein (SEQ ID NO:12) to be protective against other isolates.

Briefly, heat inactivated serum from pigs vaccinated with IgG-AVP8 was diluted 2-fold starting at 1:200 in MEM in a dilution block from Row A to Row G. Row H contained no serum. In a separate dilution block, Rotavirus A of various G and P types was diluted 1.5 fold across dilution plate starting at 6.0 Log₁₀ TCID₅₀/mL from column 1 to column 11. Column 12 contained no virus. 250 μL of virus and 250 μL of serum from corresponding wells were combined and incubated for 1 hour at 37° C. After 1 hour incubation, 100 μL the virus-serum mixture was overlayed onto a monolayer of MA104 cells and incubated at 37° C. for 72 hours and stained by IFA and read for presence of virus. The presence of virus was recorded as ‘+’ on plate and the lack of virus was recorded as ‘0’. These results were then transferred to Table 8.

The following six Rotavirus A isolates were compared with this assay; G9P[7], G9P[23], G4P[23], G3P[7], G5P[7], and G4P[7]. Results in Table 1 indicate that P type P[23] cross neutralizes P[7]. All G types that included P[7] or P[23] were also neutralizing indicating that in this assay G type was not significant in the neutralization of virus.

TABLE 8
Study Design and Results

| G9P[7] | 1 | 2 | 3 | 4 | 5 | 6 | 7 | 8 | 9 | 10 | 11 | 12 |
|---|---|---|---|---|---|---|---|---|---|---|---|---|
| serum @ 1:200 A | 0 | 0 | 0 | 0 | 0 | 0 | 0 | 0 | 0 | 0 | 0 | 0 |
| serum @ 1:400 B | 0 | 0 | 0 | 0 | 10 | 0 | 0 | 0 | 0 | 0 | 0 | 0 |
| serum @1:800 C | + | + | + | 0 | 0 | 0 | 0 | 0 | 0 | 0 | 0 | 0 |
| serum @ 1:1600 D | + | + | 0 | 0 | 0 | 0 | 0 | 0 | 0 | 0 | 0 | 0 |
| serum @ 1:3200 E | + | + | + | + | 0 | 0 | 0 | 0 | 0 | 0 | 0 | 0 |
| serum @ 1:6400 F | + | + | + | + | + | + | 0 | 0 | 0 | 0 | 0 | 0 |
| serum @ 1:12800 G | + | + | + | + | + | + | 0 | + | + | + | 0 | 0 |
| no serum H | + | + | + | + | + | + | + | + | + | + | 0 | 0 |
| Virus Dilution | 100 | 150 | 225 | 338 | 506 | 759 | 1139 | 1709 | 2563 | 3844 | 5767 | No virus |
| G9P[23] | 1 | 2 | 3 | 4 | 5 | 6 | 7 | 8 | 9 | 10 | 11 | 12 |
| serum @ 1:200 A | 0 | 0 | 0 | 0 | 0 | 0 | 0 | 0 | 0 | 0 | 0 | 0 |
| serum @ 1:400 B | 0 | 0 | 0 | 0 | 0 | 0 | 0 | 0 | 0 | 0 | 0 | 0 |
| serum @1:800 C | 0 | 0 | 0 | 0 | 0 | 0 | 0 | 0 | 0 | 0 | 0 | 0 |
| serum @ 1:1600 D | 0 | 0 | 0 | 0 | 0 | 0 | 0 | 0 | 0 | 0 | 0 | 0 |
| serum @ 1:3200 E | + | + | 0 | 0 | 0 | 0 | + | 0 | 0 | 0 | 0 | 0 |
| serum @ 1:6400 F | + | + | + | + | + | + | + | + | 0 | 0 | 0 | 0 |
| serum @ 1:12800 G | + | + | + | + | ++ | + | + | + | + | + | + | 0 |
| no serum H | + | + | + | + | + | + | + | + | + | + | + | 0 |
| Virus Dilution | 100 | 150 | 225 | 338 | 506 | 759 | 1139 | 1709 | 2563 | 3844 | 5767 | No virus |
| G4P[23] | 1 | 2 | 3 | 4 | 5 | 6 | 7 | 8 | 9 | 10 | 11 | 12 |
| serum @ 1:200 A | 0 | 0 | 0 | 0 | 0 | 0 | 0 | 0 | 0 | 0 | 0 | 0 |
| serum @ 1:400 B | 0 | 0 | 0 | 0 | 0 | 0 | 0 | 0 | 0 | 0 | 0 | 0 |
| serum @1:800 C | 0 | 0 | 0 | 0 | 0 | 0 | 0 | 0 | 0 | 0 | 0 | 0 |
| serum @ 1:1600 D | 0 | 0 | 0 | 0 | 0 | 0 | 0 | 0 | 0 | 0 | 0 | 0 |
| serum @ 1:3200 E | + | + | + | 0 | 0 | 0 | 0 | 0 | 0 | 0 | 0 | 0 |
| serum @ 1:6400 F | + | + | + | + | + | 0 | 0 | 0 | 0 | 0 | 0 | 0 |
| serum @ 1:12800 G | + | + | + | + | + | 0 | 0 | 0 | 0 | 0 | 0 | 0 |
| no serum H | + | + | + | + | + | + | + | + | + | 0 | + | 0 |
| Virus Dilution | 100 | 150 | 225 | 338 | 506 | 759 | 1139 | 1709 | 2563 | 3844 | 5767 | No virus |
| G3P[7] | 1 | 2 | 3 | 4 | 5 | 6 | 7 | 8 | 9 | 10 | 11 | 12 |
| serum @ 1:200 A | 0 | 0 | 0 | 0 | 0 | 0 | 0 | 0 | 0 | 0 | 0 | 0 |
| serum @ 1:400 B | 0 | 0 | 0 | 0 | 0 | 0 | 0 | 0 | 0 | 0 | 0 | 0 |
| serum @1:800 C | 0 | 0 | 0 | 0 | 0 | 0 | 0 | 0 | 0 | 0 | 0 | 0 |
| serum @ 1:1600 D | 0 | 0 | 0 | 0 | 0 | 0 | 0 | 0 | 0 | 0 | 0 | 0 |
| serum @ 1:3200 E | + | 0 | 0 | 0 | 0 | 0 | 0 | 0 | 0 | 0 | 0 | 0 |
| serum @ 1:6400 F | 0 | 0 | 0 | 0 | 0 | 0 | 0 | 0 | 0 | 0 | 0 | 0 |
| serum @ 1:12800 G | + | + | + | + | + | 0 | 0 | 0 | 0 | 0 | 0 | 0 |
| no serum H | + | + | + | + | + | + | + | + | + | + | 0 | 0 |
| Virus Dilution | 100 | 150 | 225 | 338 | 506 | 759 | 1139 | 1709 | 2563 | 3844 | 5767 | No virus |
| G5P[7] | 1 | 2 | 3 | 4 | 5 | 6 | 7 | 8 | 9 | 10 | 11 | 12 |
| serum @ 1:200 A | 0 | 0 | 0 | 0 | 0 | 0 | 0 | 0 | 0 | 0 | 0 | 0 |
| serum @ 1:400 B | 0 | 0 | 0 | 0 | 0 | 0 | 0 | 0 | 0 | 0 | 0 | 0 |
| serum @1:800 C | + | + | + | 0 | 0 | 0 | 0 | 0 | 0 | 0 | 0 | 0 |
| serum @ 1:1600 D | + | + | + | + | + | 0 | 0 | 0 | 0 | 0 | 0 | 0 |
| serum @ 1:3200 E | + | + | + | + | + | + | + | 0 | 0 | 0 | 0 | 0 |
| serum @ 1:6400 F | + | + | + | + | + | + | + | + | + | 0 | 0 | 0 |
| serum @ 1:12800 G | + | + | + | + | + | + | + | + | + | + | 0 | 0 |
| no serum H | + | + | + | + | + | + | + | + | + | + | + | 0 |
| Virus Dilution | 100 | 150 | 225 | 338 | 506 | 759 | 1139 | 1709 | 2563 | 3844 | 5767 | No virus |
| G4P[7] | 1 | 2 | 3 | 4 | 5 | 6 | 7 | 8 | 9 | 10 | 11 | 12 |
| serum @ 1:200 A | 0 | 0 | 0 | 0 | 0 | 0 | 0 | 0 | 0 | 0 | 0 | 0 |
| serum @ 1:400 B | + | 0 | 0 | 0 | 0 | 0 | 0 | 0 | 0 | 0 | 0 | 0 |
| serum @1:800 C | 0 | 0 | 0 | 0 | 0 | 0 | 0 | 0 | 0 | 0 | 0 | 0 |
| serum @ 1:1600 D | + | 0 | 0 | 0 | 0 | 0 | 0 | 0 | 0 | 0 | 0 | 0 |
| serum @ 1:3200 E | + | 0 | + | 0 | 0 | 0 | 0 | 0 | 0 | 0 | 0 | 0 |
| serum @ 1:6400 F | + | + | + | 0 | 0 | 0 | 0 | 0 | 0 | 0 | 0 | 0 |
| serum @ 1:12800 G | + | + | + | + | + | + | 0 | 0 | 0 | 0 | 0 | 0 |
| no serum H | + | + | + | + | + | + | + | + | + | + | + | 0 |
| Virus Dilution | 100 | 150 | 225 | 338 | 506 | 759 | 1139 | 1709 | 2563 | 3844 | 5767 | No virus |

In conclusion, animals vaccinated with IgG-AVP8 (including AVP8-IgG Fc protein (SEQ ID NO:12)) will cross neutralize rotavirus genotypes P[7] and P[23]. G type played no significant role in the neutralization of virus.

### Example 5

### Proof of Concept Experiment in Swine:

A total of 40 animals are used in this study. Pigs are randomized into four treatment groups with 10 pigs per group. Pigs are comingled throughout the study. General health observations, prescreen serum samples, and prescreen fecal samples are taken prior to treatment to confirm the health of animals, determine baseline serological response to rotavirus A, and to confirm no active rotavirus A infection prior or at the time of vaccination. On study zero (DO), animals are vaccinated intramuscularly with the following materials: T01: IgG-P[7]AVP8 vaccine (comprising the polypeptide of SEQ ID NO:12), T02: IgG-P[13]AVP8 vaccine (comprising the polypeptide of SEQ ID NO:14), T03: P[7]AVP8-IgG-P[13]AVP8 vaccine (comprising the polypeptide of SEQ ID NO:16), T04: placebo. Serum samples are taken on study days 0, 7, 14, 21, 28, 36, 42, and 49. All animals are humanely euthanized on study D49 at necropsy. Serum samples are tested by a virus neutralization assay to determine the serological response to vaccine prototypes over time. Animals vaccinated with T01 have antibodies neutralizing rotavirus genotypes P[7] and P[23], animals vaccinated with T02 have antibodies neutralizing rotavirus genotype P[13] and animals vaccinated with T03 have antibodies neutralizing rotavirus genotypes P[7], P[13] and P[23].

### Example 6

### SDS PAGE:

SDS-PAGE of protein A purified AVP8-IgG Fc protein (SEQ ID NO:12) product with and without DTT (FIG. 5 A)): The method to generate the samples for the SDS-PAGE image was briefly as follows: baculovirus harvest supernatant was inactivated with 10 mM BEI at 37° C. for 36 hours and then neutralized. The sample was then purified using protein A resin. All samples were then denatured using NuPAGE 4×LDS sample buffer (Invitrogen cat #NP0007) with either 25 mM DTT (final) or equal volume of water, and heated at 95 C for 10 minutes. Samples were run out on a 4-12% SDS-PAGE gel (Invitrogen cat #NP0335BOX) at 180V for 45 minutes and stained (eStain L1, GenScript cat #M00548-1; destain cat #M00549-1). As a result, it was found that in the lane run with the reduced (+DTT (Dithiothreitol)) sample mainly one band (monomeric AVP8-IgG Fc protein, which was considered in conjunction with the results of the Western Blot described below) was seen. In the lane run with the non-reduced sample (−DTT) additional bands were seen. The additional bands were in molecular weight ranges each being a multiple of the monomer.

### Western Blot:

Anti-swine IgG Fc fragment Western Blot (FIG. 5 B)): AVP8-IgG Fc protein (SEQ ID NO:12) product that had been produced in a bioreactor was harvested with a 1 mL sample prior to BEI addition. The sample was centrifuged at 20,000 g and 4° C. for 5 minutes, supernatant decanted to a fresh tube, and both pellet and supernatant stored at −70 C. Pellet and supernatant were thawed, pellet resuspended in 1 mL of 8M urea, then equal amounts of pellet and supernatant were run out on SDS-PAGE under reducing conditions (+DTT), and transferred to a PVDF membrane. Western blot was probed with 1:1000 dilution of HRP conjugated goat anti-swine to detect swine IgG Fc fragment.

As a result, unexpectedly no AVP8-IgG Fc protein was seen in the cell pellet sample. Instead all AVP8-IgG Fc protein (SEQ ID NO:12) was advantageously found in the cell culture supernatant sample.

### Example 7

### Generation of Consensus Sequences:

The consensus sequences of SEQ ID NO:4 (based on genotype P[6] rotavirus VP8 protein) and SEQ ID NO:5 (based on genotype P[13] rotavirus VP8 protein) were generated, as described in the following:

Sequences were compiled from publically available swine rotavirus VP4 nucleotide sequences from the NCBI Virus Variation database and internally derived rotavirus isolate sequences. Additional metadata for sequences was also compiled including metadata for: isolate name, isolate P-Type, Geographic Origin, and date of isolation when available. Nucleotide sequences were translated into protein sequences, and aligned to known VP8 proteins using MUSCLE sequence alignment software UPGMB clustering and default gap penalty parameters. Unaligned VP5 amino acids were trimmed and discarded. VP8 aligned protein sequences were imported into MEGA7 software for phylogenetic analysis and a neighbor joining phylogeny reconstruction was generated based on VP8 protein sequence. The optimal tree was computed using the Poisson correction method with bootstrap test of phylogeny (n=100) and drawn to scale with branch lengths equal to evolutionary distances in units of amino acid substitutions per site over 170 total positions. Nodes where bootstrap cluster association was greater than 70% were considered significant. Nodes with approximately 10% distance and bootstrap cluster associations greater than 70% were designated as clusters. Outlier sequences not fitting into large clusters were individually assessed for sequence quality and P-type origin. Suspected low quality sequences were removed from the analysis, while sequences from rarely observed P-types in swine rotavirus were retained. Clusters used to generate consensus sequences were selected based on desired product protection profile as well as in-vitro serum cross neutralization studies. Consensus sequences were generated by greatest frequency per aligned position, in cases where equivalent proportions of amino acids were observed in an aligned position, the amino acid residue was selected based on reported epidemiological data in conjunction with product protection profile.

### Example 8

### Challenge Studies:

The primary purpose of this study was to evaluate whether administration of a prototype vaccine, also termed “IgG #AVP8” herein, including AVP8-IgG Fc protein (SEQ ID NO:12) and a non-relevant control vaccine, termed “Placebo” herein, to conventional dams conferred passive protection to pigs against a virulent rotavirus A challenge. The prototype vaccine, was produced similarly to the production described above in Example 1, but with different volumes used for the infection and a different purification method, as described below in the section “Production of IgG #AVP8”.

A total of 20 dams were included in the study. Dams were randomized into two treatment groups and one strict control group as described in Table 9 below. Dams in T01 and T03 were comingled between three rooms. Dams in T07 were housed in a separate room. All dams were vaccinated with the appropriate material by the appropriate route as listed in Table 9. Dams in T07 remained non-vaccinated (strict control). Serum was collected from the dams periodically throughout the vaccination period and assayed for evidence of seroconversion. Fecal samples were collected prior to farrowing and screened by RT-qPCR to confirm dams were not actively shedding rotavirus prior to farrowing. General health observations were recorded on each sow daily. Farrowing was allowed to occur naturally until the sow reached gestation day 114. After this time, farrowing was induced. Piglets were enrolled into the trial at the time of farrowing. Only piglets which were healthy at birth were tagged, processed according to facility standard operating procedures, and included in the trial. When pigs were one to five days of age, they were bled, a fecal swab was collected, and pigs were challenged (excluding T07). At the time of challenge, pigs were administered an intragastric, 5 mL dose of sodium bicarbonate, then an intragastric, 1 mL dose of the challenge material. Throughout the challenge period, all animals were monitored daily for the presence of enteric disease (diarrhea, and behavior changes). Fecal samples were collected on one day post challenge (DPC1). At DPC2, all pigs in T01 and T03 were euthanized. Intestinal sections were collected for microscopic and immunohistological evaluation.

TABLE 9
Study Design

| | | | | | | Piglet | |
|---|---|---|---|---|---|---|---|
| | | | | Sow vaccination | | challenge | |
| | N | N | | (6 & 2 wks pre-farrow) | | (DPC0; 1-5 | |
| Group | (dams) | (piglets) | Room | Description | Route/dose* | days of age) | Necropsy |
| T01 | 8 | 67 | Comingled | Placebo | 2 mL IM | Rotavirus A | DPC2 |
| T03 | 8 | 72 | between | IgG#AVP8 | 2 mL IM | P[7] tissue | |
| | | | rooms CC1, | | | homogenate 1:2 dilution | |
| | | | CC2, CC3 | | | 1 mL dose | |
| | | | | | | intragastrically | |
| T07 | 4 | 35 | CB8 | Strict | Not | None | Not |
| | | | | control | applicable | | applicable |

*IM = intramuscular

Throughout the study, serum VN titers in dams from T07 (strict control) increased by less than 4-fold indicating lack of exposure and a valid study (virus neutralization was assessed as described above in Example 1 (“Protocol for virus neutralization assay”), results are shown in Table 10 and FIG. 6). During the vaccination phase, the highest mean VN titers in serum were observed in dams vaccinated with the prototype vaccine IgG #AVP8 (group T03). In this group, one dose administered at six weeks pre-farrow resulted in four-fold or greater increased titer in 6/8 animals in T03 (IgG #AVP8) by D14. None of the dams in group T01 (Placebo) had a significant increase (<2-fold) in serum VN titer during the vaccination phase. Dams colostrum VN titers: dams in group T03 (IgG #AVP8) had a higher mean VN titer in comparison to dams in group T01 (Placebo).

TABLE 10
VN results

| | | Serum | | | | | Colostrum |
|---|---|---|---|---|---|---|---|
| Group | Dam | D0 | D14 | D21 | D28 | DPC0 | DOF* |
| T01 | 887 | 160 | 320 | 320 | 453 | 320 | 160 |
| | 7768 | 320 | 160 | 320 | 320 | 320 | 1280 |
| | 7777 | 320 | 320 | 640 | 640 | 640 | 226 |
| | 7785 | 320 | 640 | 905 | 905 | 1810 | 640 |
| | 7795 | 160 | 80 | 640 | 640 | 640 | 1280 |
| | 7802 | 320 | 113 | 453 | 320 | 640 | 1280 |
| | 7813 | 320 | 640 | 640 | 905 | 453 | 905 |
| | 7821 | 160 | 320 | 320 | 160 | 113 | 320 |
| T03 | 1051 | 320 | 1280 | 320 | 1280 | 320 | 640 |
| | 7767 | 160 | 640 | 1280 | 905 | 1280 | 2560 |
| | 7772 | 160 | 1280 | 1280 | 1280 | 2560 | 2560 |
| | 7774 | 160 | 1280 | 1280 | 1280 | 2560 | 1280 |
| | 7781 | 320 | 640 | 905 | 640 | 640 | 453 |
| | 7783 | 160 | 640 | 1280 | 905 | 2560 | 2560 |
| | 7798 | 113 | 226 | 640 | 640 | 1280 | 453 |
| | 7807 | 160 | 1280 | 1280 | 453 | 1280 | 2560 |
| T07 | 886 | 226 | 80 | 160 | 1280 | 320 | 320 |
| | 889 | 160 | 320 | 320 | 320 | 453 | 1280 |
| | 7770 | 226 | 113 | 320 | 226 | 320 | 1280 |
| | 7778 | 226 | 453 | 320 | 320 | 640 | 80 |

*DOF = day of farrow

The VN titers in pig serum pre-challenge were high (>1280) in the majority of pigs in group T03 (IgG #AVP8) indicating passive transfer of immunity from dams to pigs. Conversely, the majority of titers in pigs in T02 (Placebo) were low (<1280).

In Groups T01 (Placebo) and T03 (IgG #AVP8) a pig was defined as affected if rotavirus antigen was detected by immunohistochemistry (IHC) in at least one intestinal section and the animal had an abnormal fecal score for at least one day post-challenge. The frequency distributions are listed in Table 11 below. Based on the use of this case definition, vaccination of dams at 6 and 2 weeks pre-farrow with the prototype vaccine IgG #AVP8 (group T03) prevented rotavirus associated disease in pigs following challenge with heterologous rotavirus A P[7] challenge material; preventative fraction 0.926, 95% confidence interval 0.734, 0.979.

TABLE 11
Frequency distribution of case definition

| Case Definition* | |
|---|---|
| 0 | 1 |

| Group | Total | N | % | N | % |
|---|---|---|---|---|---|
| T01 | 67 | 42 | 62.7 | 25 | 37.3 |
| T03 | 72 | 70 | 97.2 | 2 | 2.8 |

*Case definition: A pig was considered affected if one or more of the ileum or jejunum tissue samples is IHC positive (score >0) for Rotavirus A and has at least one abnormal fecal score on any one day post-challenge. A score of 0 = non-affected; 1 = affected

In conclusion, vaccination of conventional dams at six- and two-weeks prefarrow with the prototype vaccine IgG #AVP8 (comprising the polypeptide of SEQ ID NO:12) lead to high neutralizing antibody titers in sow serum and colostrum. These neutralizing antibodies were passively transmitted to pigs following birth as evidenced by detection of high titers (>1280) in the serum of pigs from vaccinated dams. The presence of high neutralizing antibody titers in the pigs lead to clinical protection. Specifically, fewer pigs born to vaccinated dams were considered affected as compared to pigs born to placebo controls.

### Production of IgG #AVP8

Two 10 L Sartorius Biostat B glass-jacketed vessels were seeded with 3 L of Sf+ cells at 1.00×10\hat{ }6 cells/mL. Three days after planting, each vessel was infected at an MOI of 0.1 and the volume of each vessel was adjusted to 8 L using Ex-cell 420 serum free medium (SAFC cat #14420C-1000 mL). The bioreactor was run at 27° C. with 100 rpm agitation, with the dissolved oxygen set at or above 40%, and a CCA overlay at 1.3 slpm. The vessel was harvested at 7 days post inoculation; fluids were centrifuged at 10,000 g at 4° C. for 20 minutes, and supernatant was 0.8/0.2 μm filtered (GE Healthcare, cat #6715-7582). The clarified supernatant (8 L/vessel) was inactivated with 5 mM BEI for three days at 37° C. in the Sartorius Biostat B glass-jacketed vessels. Following inactivation, residual BEI was neutralized with sodium thiosulfate. Following neutralization, 7000 mL was concentrated approximately 10× using a 10 kDa hollow fiber filter (GE, cat #UFP-10-C-5A) to 700 mL. Concentrated material was diafiltrated with 5 volumes (3500 mL) of 1×PBS. The vaccine was formulated with 12.5% Emulsigen D, 28% concentrated material, and 59.5% 1×PBS (volume:volume).

### Example 9

### Proof of Concept Experiment in Swine:

A total of 20 animals are used in this study. Pigs are randomized into two treatment groups with 10 pigs per group. Pigs are comingled throughout the study. General health observations, prescreen serum samples, and prescreen fecal samples are taken prior to treatment to confirm the health of animals, determine baseline serological response to rotavirus C, and to confirm no active rotavirus C infection prior or at the time of vaccination. On study day zero (DO) and D28, animals are vaccinated intramuscularly with the following materials: T01: IgG-CVP8 vaccine (comprising the polypeptide of SEQ ID NO:15), T02: placebo. Serum samples are taken on study days 0, 7, 14, 21, 28, 36, and 42. All animals are humanely euthanized on study D42 at necropsy. Serum samples are tested by an ELISA to determine the serological response to vaccine prototypes over time. Animals vaccinated with T01 have a higher mean level of antibodies against rotavirus C than the animals vaccinated with T02, which do not have an increase in titer.

### Example 10

### Proof of Concept Experiment in Swine:

The primary purpose of this study was to evaluate whether administration of a prototype vaccine, also termed “IgG:AVP8 P[6,7,13]” herein, which included three AVP8-IgG Fc proteins (SEQ ID NO:12, SEQ ID NO:13, SEQ ID NO:14) and a non-relevant control vaccine, termed “Placebo” herein, to conventional dams conferred passive protection to pigs against a virulent rotavirus A challenge. The components of the prototype vaccine were produced similarly to the production described above in Example 1 and were subsequently mixed, as described below in the section “Production of IgG:AVP8 P[6,7,13]”.

A total of 32 dams were included in the study. Dams were randomized into two treatment groups and one strict control group as described in Table 12 below. Dams in T01 and T03 were commingled between seven rooms. Dams in T04 were housed in a separate room. All dams were vaccinated with the appropriate material by the appropriate route as listed in Table 12. Dams in T04 remained non-vaccinated (strict control). Serum was collected from the dams periodically throughout the vaccination period and assayed for evidence of seroconversion by virus neutralization (VN) and enzyme-linked immunosorbent assay (ELISA). Fecal samples were collected prior to farrowing and screened by RT-qPCR to confirm dams were not actively shedding rotavirus prior to farrowing. Within this study, RT-qPCR was conducted as described above in Example 2 (“Protocol for Rota A qRT-PCR”). General health observations were recorded on each sow daily. Farrowing was allowed to occur naturally until the sow reached gestation day 115. After this time, farrowing was induced. Piglets were enrolled into the trial at the time of farrowing. Only piglets which were healthy at birth were tagged, processed according to facility standard operating procedures, and included in the trial. When pigs were two to five days of age, they were bled, a fecal swab was collected, and pigs were challenged (excluding T04). At the time of challenge, pigs were administered an intragastric, 5 mL dose of sodium bicarbonate, then an intragastric, 1 mL dose of the challenge material. Throughout the challenge period, all animals were monitored daily for the presence of enteric disease (diarrhea, and behavior changes). Fecal samples were collected on one day post challenge (DPC1) and DPC3. At DPC3, all pigs in T01 and T03 were euthanized. Intestinal sections were collected for microscopic and immunohistological evaluation.

TABLE 12
Study Design

| | | Dam vaccination | | Piglet | |
|---|---|---|---|---|---|
| | | (6 & 2 wks pre-farrow) | | challenge | |
| | N | | Route/ | (DPC0; 2-5 | |
| Group | (dams) | Description | dose* | days of age) | Necropsy |
| T01 | 14 | Placebo | 2 mL IM | Rotavirus A | DPC3 |
| T03 | 14 | IgG:AVP8 | 2 mL IM | P[7] tissue | |
| | | P[6, 7, 13] | | homogenate | |
| | | | | 1:2 dilution | |
| | | | | 1 mL dose | |
| | | | | intragastrically | |
| T04 | 4 | Strict | Not | None | Not |
| | | control | applicable | | applicable |

*IM = intramuscular

Throughout the study, serum VN titers in dams from T04 (strict control) increased by 4-fold or less indicating lack of exposure and a valid study (virus neutralization was assessed as described below (“Protocol for virus neutralization assay”), results are shown in Table 13 and FIG. 7). None of the dams in group T01 (Placebo) had a significant increase (<2-fold) in serum VN titer during the vaccination phase. VN titers in serum from dams vaccinated with the prototype vaccine IgG:AVP8 P[6,7,13] (Group T03) were significantly higher (p<0.01) than dams vaccinated with a placebo (Group T01) at D14, D21, D27, and D47. Mean colostrum VN titers from dams in group T03 (IgG:AVP8 P[6,7,13]) were also significantly higher in comparison to dams in group T01 (Placebo).

TABLE 13
VN results

| | | Serum | | | | | Colostrum |
|---|---|---|---|---|---|---|---|
| Group | Dam | D0 | D14 | D21 | D28 | D47 | DOF* |
| T01 | 1054 | 226 | 320 | 226 | 226 | 160 | 320 |
| | 1154 | 80 | 80 | 160 | 160 | 113 | 320 |
| | 1155 | 113 | 113 | 160 | 226 | 160 | 320 |
| | 1160 | 113 | 453 | 226 | 226 | 80 | 226 |
| | 1162 | 226 | 320 | 640 | 320 | 320 | 640 |
| | 1163 | 160 | 226 | 226 | 160 | 320 | 1280 |
| | 8093 | 80 | 80 | 160 | 130 | 160 | 453 |
| | 8101 | 320 | 160 | 320 | 320 | 320 | 320 |
| | 8105 | 320 | 1280 | 453 | 905 | 453 | 640 |
| | 8107 | 160 | 320 | 320 | 320 | 226 | 40 |
| | 8120 | 226 | 320 | 226 | 320 | 320 | 640 |
| | 8121 | 226 | 320 | 160 | 640 | 226 | 320 |
| | 8127 | 160 | 160 | 320 | 226 | 160 | 160 |
| | 8131 | 320 | 320 | 453 | 453 | 640 | 640 |
| T03 | 1045 | 1280 | 1280 | 1280 | 1280 | 2560 | 640 |
| | 1053 | 320 | 2560 | 1280 | 1280 | 1280 | 2560 |
| | 1055 | 320 | 905 | 1280 | 1280 | 640 | 3620 |
| | 1158 | 320 | 453 | 905 | 1280 | 640 | 2560 |
| | 1159 | 160 | 320 | 905 | 640 | 1810 | 5120 |
| | 1161 | 320 | 320 | 640 | 320 | 1280 | 1280 |
| | 8100 | 226 | 2560 | 1810 | 622 | 10240 | 14482 |
| | 8102 | 320 | 640 | 905 | 640 | 5120 | 3620 |
| | 8103 | 320 | 1280 | 640 | 453 | 1280 | 1810 |
| | 8108 | 320 | 1810 | 1280 | 640 | 1280 | 2560 |
| | 8117 | 453 | 320 | 640 | 905 | 640 | 2560 |
| | 8118 | 320 | 905 | 1280 | 640 | 1280 | 905 |
| | 8122 | 160 | 320 | 640 | 320 | 1280 | 640 |
| | 8130 | 113 | 640 | 640 | 905 | 1280 | 10240 |
| T04 | 1046 | 320 | 640 | 640 | 640 | 1280 | 453 |
| | 1164 | 320 | 320 | 226 | 320 | 320 | 1280 |
| | 8123 | 640 | 640 | 1280 | 640 | 905 | 640 |
| | 8126 | 226 | 320 | 640 | 320 | 640 | 2560 |

The VN titers in pig serum pre-challenge were high (>1280) in the majority of pigs in group T03 (IgG:AVP8 P[6,7,13]) indicating passive transfer of immunity from dams to pigs. Conversely, none of the VN titers in pigs in T01 (Placebo) were high (>1280).

Similar to the VN data, an increase in the amount of anti-Rotavirus AVP8 P[6] and anti-Rotavirus AVP8 P[13] antibodies was detected in dams vaccinated with the IgG:AVP8 P[6,7,13] prototype (Group T03) by D47 but not in dams vaccinated with a placebo (Group T01). These assays were conducted as described below (“Protocol for IgG:AVP8 P[13] ELISA” and “Protocol for IgG:AVP8 P[6] ELISA”, respectively). Results are shown in FIG. 8 and FIG. 9.

In Groups T01 (Placebo) and T03 (IgG:AVP8 P[6,7,13]) a pig was defined as affected if rotavirus antigen was detected by immunohistochemistry (IHC) in at least one intestinal section and the animal had an abnormal fecal score for at least one day post-challenge. The frequency distributions are listed in Table 14 below. Based on the use of this case definition, vaccination of dams at 6 and 2 weeks pre-farrow with the prototype vaccine IgG:AVP8 P[6,7,13] (Group T03) prevented rotavirus associated disease in pigs following challenge with heterologous rotavirus A P[7] challenge material; preventative fraction 0.567, 95% confidence interval—0.086, 0.827.

TABLE 14
Frequency distribution of case definition

| Case Definition* | |
|---|---|
| 0 | 1 |

| Group | N | % | N | % | Total |
|---|---|---|---|---|---|
| T01 | 78 | 60.9 | 50 | 39.1 | 128 |
| T03 | 113 | 83.1 | 23 | 16.9 | 136 |

*Case definition: A pig was considered affected if one or more of the ileum or jejunum tissue samples is IHC positive (score >0) for Rotavirus A and has at least one abnormal fecal score on any one day post-challenge. A score of 0 = non-affected; 1 = affected

In conclusion, vaccination of conventional dams at six- and two-weeks pre-farrow with the prototype vaccine IgG:AVP8 P[6,7,13] (comprising the polypeptides of SEQ ID NO:12, SEQ ID NO:13, SEQ ID NO:14) lead to high neutralizing antibody titers in sow serum and colostrum. These neutralizing antibodies were passively transmitted to pigs following birth as evidenced by detection of high titers (>1280) in the serum of pigs from vaccinated dams. The presence of high neutralizing antibody titers in the pigs lead to clinical protection. Specifically, fewer pigs born to vaccinated dams were considered affected as compared to pigs born to placebo controls.

### Production of IgG:AVP8 P[6,7,13]

The prototype vaccine contained three fractions. The production of each fraction was done separately and is described below. The vaccine was formulated with 12.0% Emulsigen D, 7% IgG:AVP8 P[7] fraction, 4.4% IgG:AVP8 P[6] fraction, 1.8% IgG:AVP8 P[13] fraction, and 47.8% 1×PBS (volume:volume).

IgG:AVP8 P[7] fraction: One 10 L Sartorius Biostat B glass-jacketed vessels was seeded with 3 L of Sf+ cells at 0.75×10\hat{ }6 cells/mL. Two days after planting, the vessel was infected at an MOI of 0.1 and the volume of the vessel was adjusted to 8 L using Ex-cell 420 serum free medium (SAFC cat #14420C-1000 mL). The bioreactor was run at 27° C. with 100 rpm agitation, with the dissolved oxygen set at or above 40%, and a CCA overlay at 1.3 slpm. The vessel was harvested at 7 days post inoculation; fluids were centrifuged at 10,000 g at 4° C. for 20 minutes, and supernatant was 0.8/0.2 μm filtered (GE Healthcare, cat #6715-7582). The clarified supernatant (8 L/vessel) was inactivated with 5 mM BEI for three days at 37° C. in the Sartorius Biostat B glass-jacketed vessels. Following inactivation, residual BEI was neutralized with sodium thiosulfate. Following neutralization, approximately 7000 mL was concentrated approximately 10× using a 10 kDa hollow fiber filter (GE, cat #UFP-10-C-5A) to 700 mL. Concentrated material was diafiltrated with 5 volumes (3500 mL) of 1×PBS.

IgG:AVP8 P[6] fraction: One 10 L Sartorius Biostat B glass-jacketed vessels was seeded with 3 L of Sf+ cells at 0.75×10\hat{ }6 cells/mL. Two days after planting, the vessel was infected at an MOI of 0.1 and the volume of the vessel was adjusted to 8 L using Ex-cell 420 serum free medium (SAFC cat #14420C-1000 mL). The bioreactor was run at 27° C. with 100 rpm agitation, with the dissolved oxygen set at or above 40%, and a CCA overlay at 1.3 slpm. The vessel was harvested at 8 days post inoculation; fluids were centrifuged at 10,000 g at 4° C. for 20 minutes, and supernatant was 0.8/0.2 μm filtered (GE Healthcare, cat #6715-7582). The clarified supernatant (8 L/vessel) was inactivated with 10 mM BEI for 24 hours at 37° C. in the Sartorius Biostat B glass-jacketed vessels. Following inactivation, residual BEI was neutralized with sodium thiosulfate. Following neutralization, approximately 7000 mL was concentrated approximately 10× using a 10 kDa hollow fiber filter (GE, cat #UFP-10-C-5A) to 600 mL. Concentrated material was diafiltrated with 5 volumes (3500 mL) of 1×PBS.

IgG:AVP8 P[13] fraction: One 10 L Sartorius Biostat B glass-jacketed vessels was seeded with 3 L of Sf+ cells at 0.75×10\hat{ }6 cells/mL. Two days after planting, the vessel was infected at an MOI of 0.1 and the volume of the vessel was adjusted to 8 L using Ex-cell 420 serum free medium (SAFC cat #14420C-1000 mL). The bioreactor was run at 27° C. with 100 rpm agitation, with the dissolved oxygen set at or above 40%, and a CCA overlay at 1.3 slpm. The vessel was harvested at 7 days post inoculation; fluids were centrifuged at 10,000 g at 4° C. for 20 minutes, and supernatant was 0.8/0.2 μm filtered (GE Healthcare, cat #6715-7582). The clarified supernatant (8 L/vessel) was inactivated with 10 mM BEI for 24 hours at 37° C. in the Sartorius Biostat B glass-jacketed vessels. Following inactivation, residual BEI was neutralized with sodium thiosulfate. Following neutralization, approximately 7000 mL was concentrated approximately 10× using a 10 kDa hollow fiber filter (GE, cat #UFP-10-C-5A) to 750 mL. Concentrated material was diafiltrated with 5 volumes (3500 mL) of 1×PBS.

### Protocol for IgG:AVP8 P[13] ELISA:

Nuc MaxiSorb 96-well ELISA plates (Thermo) were coated with Rotavirus A VP8 P[13] purified protein diluted in 1×PBS 1:10. Plates were incubated at 37° C. for 1 hour. Following incubation, plates were washed using 1×PBST and then blocked with 3% BSA in PBST for 1 hour at 37° C. Following washing, 100 μL of test sera diluted to a final dilution of 1:512 in 1×PBS were added to plates and incubated for 1 hour at 37° C. Following washing, wells were coated with 100 μl of a 1:25,000 dilution of horse-radish peroxidase (HRP)-conjugated-goat-anti-swine-IgG antibody (Jackson ImmunoResearch) and incubated for one hour at 37° C. Following washing, the plate was developed with 3,5,3′,5′-tetramethylbenzidine (1-Step Ultra TMB-ELISA, Thermo) for 10 minutes at room temperature and the reaction was stopped with stop solution (Sigma) before optical density (OD) measurement at 450 nm. Samples, including a positive and negative control, were run in duplicate wells and results are reported as the average of (sample−negative control)-to-(positive−negative control) ratio (S−N)/(P−N).

### Protocol for IgG:AVP8 P[6] ELISA:

Nuc MaxiSorb 96-well ELISA plates (Thermo) were coated with Rotavirus A VP8 P[6] purified protein diluted in 1×PBS 1:10. Plates were incubated at 37° C. for 1 hour. Following incubation, plates were washed using 1×PBST and then blocked with 3% BSA in PBST for 1 hour at 37° C. Following washing, 100 μL of test sera diluted to a final dilution of 1:512 in 1×PBS were added to plates and incubated for 1 hour at 37° C. Following washing, wells were coated with 100 μl of a 1:25,000 dilution of horse-radish peroxidase (HRP)-conjugated-goat-anti-swine-IgG antibody (Jackson ImmunoResearch) and incubated for one hour at 37° C. Following washing, the plate was developed with 3,5,3′,5′-tetramethylbenzidine (1-Step Ultra TMB-ELISA, Thermo) for 10 minutes at room temperature and the reaction was stopped with stop solution (Sigma) before optical density (OD) measurement at 450 nm. Samples, including a positive and negative control, were run in duplicate wells and results are reported as the average of (sample−negative control)-to-(positive−negative control) ratio (S−N)/(P−N).

### Protocol for Virus Neutralization Assay

All serum and milk samples were heat inactivated at 56° C. for 30 minutes. Samples were serially diluted from 1:40 through 1:40,960 in rotavirus growth media (MEM+2.5% HEPES+0.3% Tryptose phosphate broth+0.02% yeast+10 μg/mL trypsin). Antibiotics were added to the media for milk sample testing. Rotavirus A isolate (titer approximately 6.5 log TCID₅₀/mL) was diluted 1:78,000 into rotavirus growth media. A total of 250 μl of the diluted sample was added to 250 μl of the diluted virus; the mixture was incubated at 37° C.±5% CO₂ for one hour. Growth media was aseptically removed from three-four day old 96-well plates planted with MA104 cells. Following incubation, 200 μl of the virus-serum mixture was transferred to the cell culture plates. Cells were incubated at 37° C.±5% CO₂ for 3-4 days. The stock and diluted virus were titrated on the day of use to confirm the dilution used in the assay. Following incubation, the supernatant was discarded. For fixation, 100 μL/well of 50%/50% acetone/methanol was added. Plates were incubated at room temperature for 15 minutes, air-dried, then rehydrated with 100 μL/well 1×PBS. The primary antibody (Rabbit anti-Rotavirus A polyclonal serum, internally generated) was diluted 1:1000 in 1×PBS. 100 μL/well of the diluted primary antibody was added and plates were incubated at 37° C.±5% CO₂ for one hour. Following incubation, plates were washed twice with 100 μL/well of 1×PBS. The secondary antibody (Jackson ImmunoResearch FITC labeled goat-anti-rabbit IgG cat #111-095-003) was diluted 1:100 in 1×PBS. 100 μL/well of the diluted secondary antibody was added and plates were incubated at 37° C.±5% CO₂ for one hour. Following incubation, plates were washed twice with 100 μL/well of 1×PBS. Plates were read for the presence of fluorescence using an ultraviolet microscope. The assay was considered valid if the titer (generated using the Reed-Muench method) of the diluted virus was found to be 2.8±0.5 log TCID₅₀/mL. In addition, known positive and negative samples were included in each assay as a control. Serum titers were reported as the highest dilution in which no staining was observed.

### Example 11

### Proof of Concept Experiment in Swine:

The primary purpose of this study is to evaluate whether administration of a prototype vaccine, also termed “IgG:AVP8 P[6,7,13]+IgG:CVP8” herein, which includes three AVP8-IgG Fc proteins (SEQ ID NO:12, SEQ ID NO:13, SEQ ID NO:14) and one CVP8-IgG Fc protein (SEQ ID NO:15) and a non-relevant control vaccine, termed “Placebo” herein, to conventional dams confers passive protection to pigs against a virulent rotavirus A challenge. A total of 24 dams are included in the study. Dams are randomized into two treatment groups and one strict control group as described in Table 15 below. Dams in T01 and T02 are comingled between three rooms. Dams in T03 are housed in a separate room. All dams are vaccinated with the appropriate material by the appropriate route as listed in Table 15. Dams in T03 remain non-vaccinated (strict control). Serum is collected from the dams periodically throughout the vaccination period and assayed for evidence of seroconversion. Fecal samples are collected prior to farrowing and screened by RT-qPCR to confirm dams are not actively shedding rotavirus prior to farrowing. General health observations are recorded on each sow daily. Farrowing is allowed to occur naturally until the sow reaches gestation day 114. After this time, farrowing is induced. Piglets are enrolled into the trial at the time of farrowing. Only piglets which are healthy at birth are tagged, processed according to facility standard operating procedures, and included in the trial. When pigs ware one to five days of age, they are bled, a fecal swab is collected, and pigs are challenged (excluding T03). At the time of challenge, pigs are administered an intragastric, 5 mL dose of sodium bicarbonate, then an intragastric, 1 mL dose of the challenge material. Throughout the challenge period, all animals are monitored daily for the presence of enteric disease (diarrhea, and behavior changes). Fecal samples are collected on one day post challenge (DPC1). At DPC3, all pigs in T01 and T02 are euthanized. Intestinal sections are collected for microscopic and immunohistological evaluation. Dams in group T01 (IgG:AVP8 P[6,7,13]+IgG:CVP8) have a higher mean level of antibodies against rotavirus A genotype P[6], P[7], and P[13] and rotavirus C than the animals vaccinated with T02 (Placebo), which do not have an increase in titer. Dams in group T01 (IgG:AVP8 P[6,7,13]+IgG:CVP8) have fewer affected piglets (as determined by clinical signs and immunohistological evaluation) as compared to dams in group T02 (Placebo).

TABLE 15
Study Design

| | | Dam vaccination | | Piglet | |
|---|---|---|---|---|---|
| | | (6 & 2 wks pre-farrow) | | challenge | |
| | N | | Route/ | (DPC0; 1-5 | |
| Group | (dams) | Description | dose* | days of age) | Necropsy |
| T01 | 10 | IgG:AVP8 | 2 mL IM | Rotavirus A | DPC2 |
| | | P[6, 7, 13] + | | P[7] tissue | |
| | | IgG:CVP8 | | homogenate | |
| T02 | 10 | Placebo | 2 mL IM | 1:2 dilution | |
| | | | | 1 mL dose | |
| | | | | intragastrically | |
| T03 | 4 | Strict | Not | None | Not |
| | | control | applicable | | applicable |

*IM = intramuscular

### LIST OF FIGURES

FIG. 1: Serum IgG response of pigs, either vaccinated with AVP8-IgG Fc protein formulated with Emulsigen D (termed “AVP8-IgG” in the labelling) or with Placebo (“Non-relevant control”), directed against porcine rotavirus A.

FIG. 2: Results of a VN (virus neutralization) assay conducted for detecting and quantifying antibodies being capable to neutralize porcine rotavirus A virus, in samples of pigs vaccinated with AVP8-IgG Fc protein formulated with Emulsigen D (termed “AVP8-IgG” in the labelling) or with Placebo (“Non-relevant control”).

FIG. 3: Mean VN titers against rotavirus in sow serum by group and study day, wherein study days DO and D28 represent the time points “six weeks and two weeks pre-farrow” (i.e. when investigational products were administered to study group T02 and T04, respectively) and study days D7, D28 and D35 represent the time points “five weeks, two weeks and one week pre-farrow” (i.e. when Commercial vaccine was administered to T06).

FIG. 4: Group median log rotavirus A RNA genomic copies (gc)/mL in feces by study day.

FIG. 5: A) SDS-PAGE of protein A purified AVP8-IgG Fc protein (SEQ ID NO:12) product samples being either reduced with Dithiothreitol (“+DTT”) or non-reduced (“−DTT”); B) Western Blot of AVP8-IgG Fc protein (SEQ ID NO:12) bioreactor product, wherein a sample was centrifuged to separate a cell pellet fraction (“Pellet”) and a supernatant fraction (“Supernatant”), which after a freeze-thaw process were run out on SDS-PAGE under reducing conditions (+DTT), transferred to a PVDF membrane and probed with HRP conjugated goat anti-swine to detect swine IgG Fc fragment.

FIG. 6: Mean VN titers against rotavirus in sow serum by group and study day, wherein study days DO and D28 represent the time points “six weeks and two weeks pre-farrow” (i.e. when investigational products were administered to study group T01 and T03, respectively).

FIG. 7: Mean VN titers against rotavirus in sow serum by group and study day, wherein study day DO represents the time point of the first administration of the investigational products to study group T01 and T03, respectively.

FIG. 8: Results of “IgG:AVP8 P[6] ELISA”, wherein ELISA plates coated with Rotavirus A VP8 P[6] protein were incubated with diluted test sera, and wherein anti-Rotavirus AVP8 P[6] antibodies were then detected by using HRP-conjugated-goat-anti-swine-IgG antibody.

FIG. 9: Results of “IgG:AVP8 P[13] ELISA”, wherein ELISA plates coated with Rotavirus A VP8 P[13] protein were incubated with diluted test sera, and wherein anti-Rotavirus AVP8 P[13] antibodies were then detected by using HRP-conjugated-goat-anti-swine-IgG antibody.

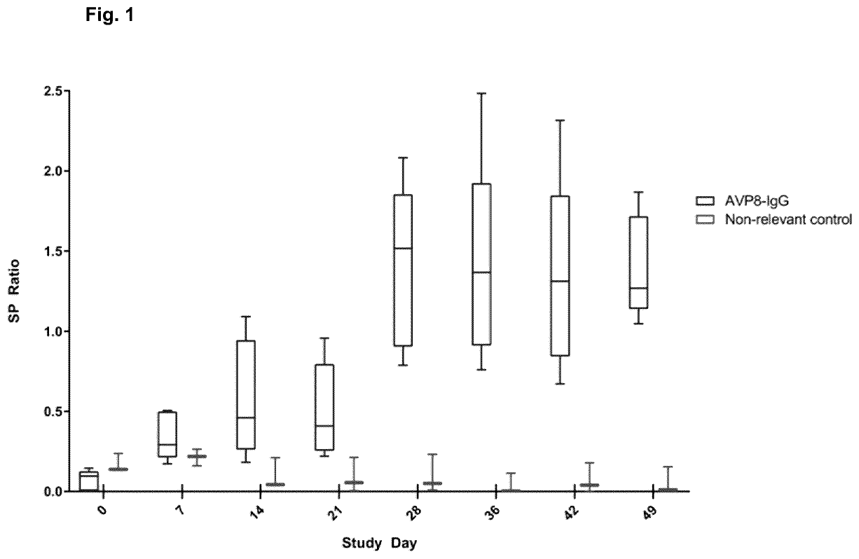

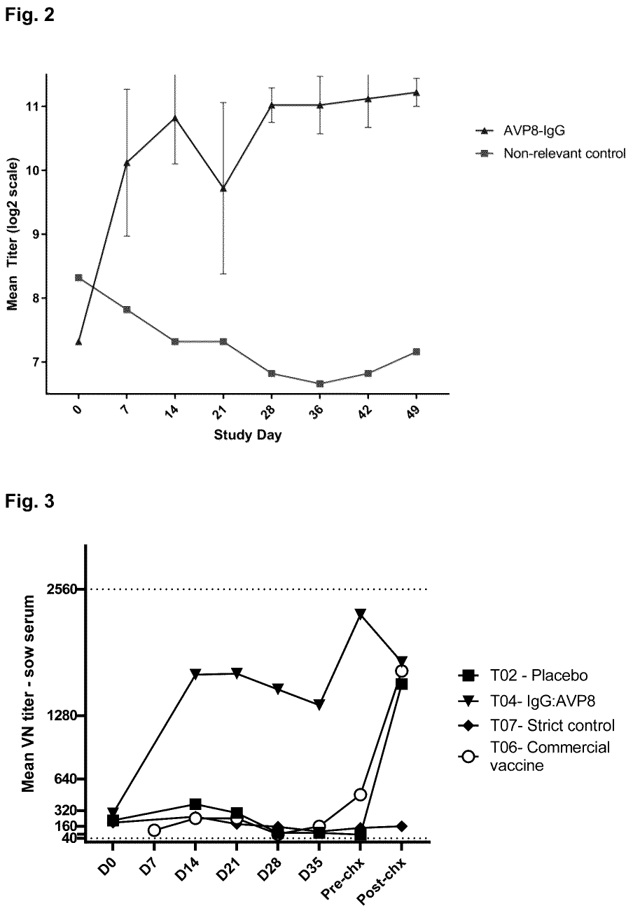

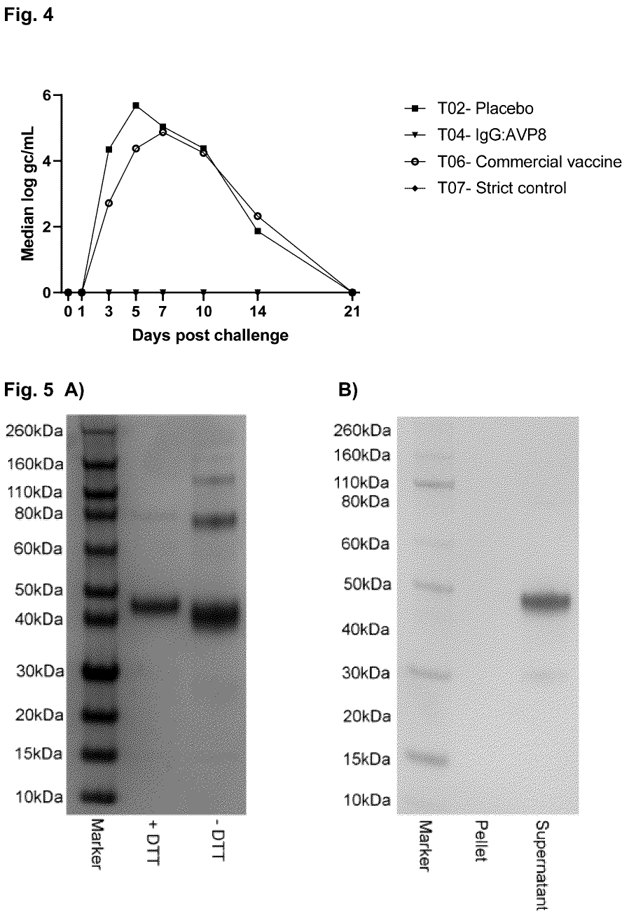

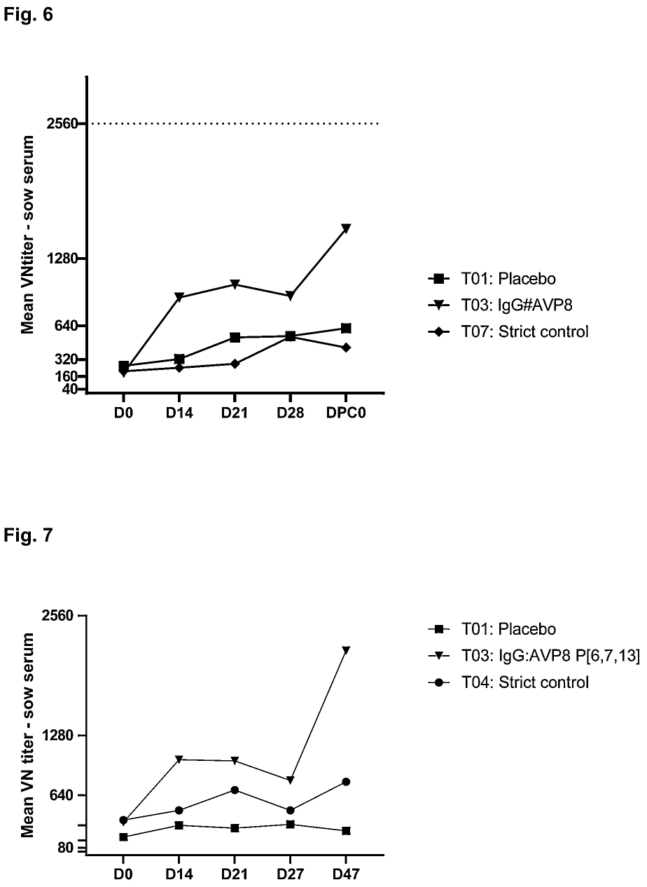

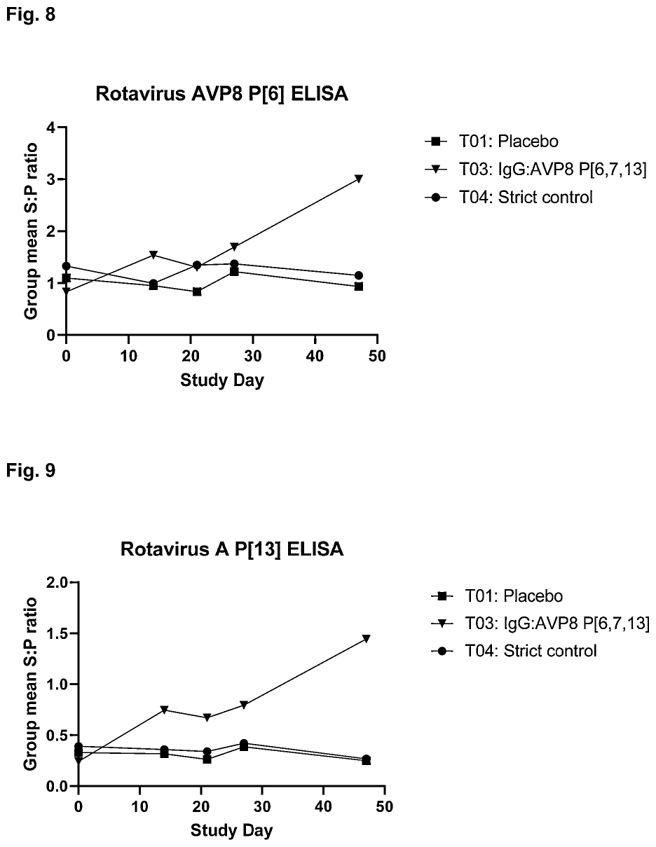

### IN THE SEQUENCE LISTING/SOURCE AND GEOGRAPHICAL ORIGIN (WHERE APPLICABLE)

SEQ ID NO:1 corresponds to the sequence of a (genotype P[7]) rotavirus VP8 protein, sourced from a farm in North Carolina, USA,
SEQ ID NO:2 corresponds to the sequence of a lectin-like domain of a (genotype P[7]) rotavirus VP8 protein, sourced from a farm in North Carolina, USA,
SEQ ID NO:3 corresponds to the sequence of an immunogenic fragment of a (genotype P[7]) rotavirus VP8 protein, sourced from a farm in North Carolina, USA,
SEQ ID NO:4 corresponds to the sequence of an immunogenic fragment of a rotavirus VP8 protein, i.e. a consensus sequence of a portion of rotavirus VP8 protein (based on genotype P[6])),
SEQ ID NO:5 corresponds to the sequence of an immunogenic fragment of a rotavirus VP8 protein, i.e. a consensus sequence of a portion of an immunogenic fragment of rotavirus VP8 protein (based on genotype P[13]),
SEQ ID NO:6 corresponds to the sequence of an immunogenic fragment of a rotavirus C VP8 protein,
SEQ ID NO:7 corresponds to the sequence of a swine IgG Fc fragment,
SEQ ID NO:8 corresponds to the sequence of a guinea pig IgG Fc fragment,
SEQ ID NO:9 (Gly-Gly-Ser) corresponds to the sequence of a linker moiety,
SEQ ID NO:10 corresponds to the sequence of a linker moiety,
SEQ ID NO:11 corresponds to the sequence of a linker moiety,
SEQ ID NO:12 corresponds to the sequence of a polypeptide (fusion protein) which comprises the sequences of SEQ ID NO:3, SEQ ID NO:9 (Gly-Gly-Ser), and SEQ ID NO:7,
SEQ ID NO:13 corresponds to the sequence of a polypeptide (fusion protein) which comprises the sequences of SEQ ID NO:4, SEQ ID NO:9 (Gly-Gly-Ser), and SEQ ID NO:7,
SEQ ID NO:14 corresponds to the sequence of a polypeptide (fusion protein) which comprises the sequences of SEQ ID NO:5, SEQ ID NO:9 (Gly-Gly-Ser), and SEQ ID NO:7,
SEQ ID NO:15 corresponds to the sequence of a polypeptide (fusion protein) which comprises the sequences of SEQ ID NO:6, SEQ ID NO:9 (Gly-Gly-Ser), and SEQ ID NO:7,
SEQ ID NO:16 corresponds to the sequence of a polypeptide (fusion protein) which comprises the sequences of SEQ ID NO:3, SEQ ID NO:9 (Gly-Gly-Ser), SEQ ID NO:7, SEQ ID NO:10, and SEQ ID NO:5,
SEQ ID NO:17 corresponds to the sequence of a polynucleotide encoding the polypeptide (fusion protein) of SEQ ID NO:12,
SEQ ID NO:18 corresponds to the sequence of a polynucleotide encoding the polypeptide (fusion protein) of SEQ ID NO:13,
SEQ ID NO:19 corresponds to the sequence of a polynucleotide encoding the polypeptide (fusion protein) of SEQ ID NO:14,
SEQ ID NO:20 corresponds to the sequence of a polynucleotide encoding the polypeptide (fusion protein) of SEQ ID NO:15,
SEQ ID NO:21 corresponds to the sequence of a polynucleotide encoding the polypeptide (fusion protein) of SEQ ID NO:16,
SEQ ID NOs:22-25: primer and probe sequences (Table 5).

The following clauses are also disclosed herein. Thus, the present disclosure further includes aspects as featured by the following clauses:

1. An immunogenic composition which comprises
(i) a polypeptide comprising
   an immunogenic fragment of a rotavirus VP8 protein, and
   an immunoglobulin Fc fragment;
and
(ii) at least one immunogenic substance, different from said polypeptide.

2. The immunogenic composition of clause 1, wherein said immunoglobulin Fc fragment is linked to the C-terminus of said immunogenic fragment of a rotavirus VP8 protein, or wherein said immunoglobulin Fc fragment is linked to the N-terminus of said immunogenic fragment of a rotavirus VP8 protein.

3. The immunogenic composition of clause 1 or 2, wherein
said immunoglobulin Fc fragment is linked to the C-terminus of said immunogenic fragment of a rotavirus VP8 protein via a linker moiety,
or wherein said immunoglobulin Fc fragment is linked to the N-terminus of said immunogenic fragment of a rotavirus VP8 protein via a linker moiety.

4. The immunogenic composition of any one of clauses 1 to 3, wherein said immunoglobulin Fc fragment is linked to the C-terminus of said immunogenic fragment of a rotavirus VP8 protein via a peptide bond between the N-terminal amino acid residue of said immunoglobulin Fc fragment and the C-terminal amino acid residue of said immunogenic fragment of a rotavirus VP8 protein,
or wherein said immunoglobulin Fc fragment is linked to the N-terminus of said immunogenic fragment of a rotavirus VP8 protein via a peptide bond between the C-terminal amino acid residue of said immunoglobulin Fc fragment and the N-terminal amino acid residue of said immunogenic fragment of a rotavirus VP8 protein.

5. The immunogenic composition of any one of clauses 1 to 4, wherein said immunoglobulin Fc fragment is linked to the C-terminus of said immunogenic fragment of a rotavirus VP8 protein.

6. The immunogenic composition of any one of clauses 1 to 5, wherein said immunoglobulin Fc fragment is linked to the C-terminus of said immunogenic fragment of a rotavirus VP8 protein via a linker moiety,
or wherein said immunoglobulin Fc fragment is linked to the C-terminus of said immunogenic fragment of a rotavirus VP8 protein via a peptide bond between the N-terminal amino acid residue of said immunoglobulin Fc fragment and the C-terminal amino acid residue of said immunogenic fragment of a rotavirus VP8 protein.

7. The immunogenic composition of any one of clauses 1 to 6, wherein said polypeptide is a fusion protein.

8. The immunogenic composition of any one of clauses 1 to 7, wherein said polypeptide is a fusion protein of the formula x-y-z, wherein
x consists of an immunogenic fragment of a rotavirus VP8 protein;
y is a linker moiety; and
z is an immunoglobulin Fc fragment.

9. The immunogenic composition of any one of clauses 1 to 8, wherein said immunogenic fragment of a rotavirus VP8 protein is capable of inducing an immune response against rotavirus in a subject to whom said immunogenic fragment of a rotavirus VP8 protein is administered.

10. The immunogenic composition of any one of clauses 1 to 9, wherein said immunogenic fragment of a rotavirus VP8 protein is 50 to 200, preferably 140 to 190 amino acid residues, in length.

11. The immunogenic composition of any one of clauses 1 to 10, wherein said rotavirus is porcine rotavirus.

12. The immunogenic composition of any one of clauses 1 to 11, wherein said rotavirus is selected from the group consisting of rotavirus A and rotavirus C.

13. The immunogenic composition of any one of clauses 1 to 12, wherein said rotavirus is rotavirus A.

14. The immunogenic composition of any one of clauses 1 to 13, wherein said immunogenic fragment of a rotavirus VP8 protein comprises the lectin-like domain of a rotavirus VP8 protein.

15. The immunogenic composition of any one of clauses 1 to 14, wherein said immunogenic fragment of a rotavirus VP8 protein is an N-terminally extended lectin-like domain of a rotavirus VP8 protein, wherein the N-terminal extension is 1 to 20 amino acid residues, preferably 5 to 15 amino acid residues, in length.

16. The immunogenic composition of clause 14 or 15, wherein the lectin-like domain of a rotavirus VP8 protein consists of the amino acid sequence of the amino acid residues 65-224 of a rotavirus VP8 protein.

17. The immunogenic composition of clause 15 or 16, wherein the amino acid sequence of said N-terminal extension is the amino acid sequence of the respective length flanking the N-terminal amino acid residue of the lectin-like domain in the amino acid sequence of the rotavirus VP8 protein.

18. The immunogenic composition of any one of clauses 1 to 17, wherein said immunogenic fragment of a rotavirus VP8 protein consists of the amino acid sequence of
the amino acid residues 60-224, the amino acid residues 59-224, the amino acid residues 58-224, the amino acid residues 57-224, the amino acid residues 56-224, the amino acid residues 55-224, the amino acid residues 54-224, the amino acid residues 53-224, the amino acid residues 52-224, the amino acid residues 51-224, the amino acid residues 50-224, or the amino residues 49-224,
of a rotavirus VP8 protein.

19. The immunogenic composition of any one of clauses 1 to 18, wherein said immunogenic fragment of a rotavirus VP8 protein consists of the amino acid sequence of the amino acid residues 57-224 of a rotavirus VP8 protein.

20. The immunogenic composition of any one of clauses 16 to 19, wherein the numbering of said amino acid residues refers to the amino acid sequence of a wild-type rotavirus VP8 protein, in particular of a wild-type rotavirus A VP8 protein, and wherein said wild-type rotavirus VP8 is preferably the protein set forth in SEQ ID NO:1.

21. The immunogenic composition of any one of clauses 1 to 20, wherein said rotavirus is selected from the group consisting of genotype P[7] rotavirus, genotype P[6] rotavirus and genotype P[13] rotavirus.

22. The immunogenic composition of any one of clauses 1 to 21, wherein the rotavirus VP8 protein comprises or consists of an amino acid sequence having at least 90%, preferably at least 95%, more preferably at least 98% or still more preferably at least 99% sequence identity with the sequence of SEQ ID NO:1.

23. The immunogenic composition of any one of clauses 14 to 22, wherein the lectin-like domain of a rotavirus VP8 protein consists of an amino acid sequence having at least 90%, preferably at least 95%, more preferably at least 98% or still more preferably at least 99% sequence identity with the sequence of SEQ ID NO:2.

24. The immunogenic composition of any one of clauses 1 to 23, wherein the immunogenic fragment of a rotavirus VP8 protein consists of an amino acid sequence having at least 90%, preferably at least 95%, more preferably at least 98% or still more preferably at least 99% sequence identity with the sequence of SEQ ID NO:3.

25. The immunogenic composition of any one of clauses 1 to 24, wherein the immunogenic fragment of a rotavirus VP8 protein consists of or is a consensus sequence of a portion of a rotavirus VP8 protein, in particular of a portion of a rotavirus A VP8 protein, and wherein said consensus sequence of a portion of a rotavirus VP8 protein is preferably obtainable by a method comprising the steps of:
translating a plurality of nucleotide sequences encoding a portion of a rotavirus VP8 protein into amino acid sequences,
aligning said amino acid sequences to known rotavirus VP8 proteins, preferably by using MUSCLE sequence alignment software UPGMB clustering and default gap penalty parameters,
subjecting said aligned sequences to a phylogenetic analysis and generating a neighbor joining phylogeny reconstruction based on rotavirus VP8 protein sequence, in particular importing said aligned amino acid sequences into MEGA7 software for phylogenetic analysis and generating a neighbor joining phylogeny reconstruction based on rotavirus VP8 protein sequence,
computing the optimal tree using the Poisson correction method with bootstrap test of phylogeny (n=100),
drawing the optimal tree to scale with branch lengths equal to evolutionary distances in units of amino acid substitutions per site over 170 total positions,
considering nodes where bootstrap cluster association is greater than 70% as significant,
designating nodes with approximately 10% distance and bootstrap cluster associations greater than 70% as clusters, and
selecting a cluster and generating the consensus sequences by identifying the greatest frequency per aligned position within the cluster,
and optionally, in cases where equivalent proportions of amino acids are observed in an aligned position, selecting the amino acid residue based on reported epidemiological data in conjunction with a predefined product protection profile.

26. The immunogenic composition of any one of clauses 1 to 25, wherein the immunogenic fragment of a rotavirus VP8 protein consists of an amino acid sequence having at least 90%, preferably at least 95%, more preferably at least 98% or still more preferably at least 99% sequence identity with a sequence selected from the group consisting of SEQ ID NO:4 and SEQ ID NO:5.

27. The immunogenic composition of any one of clauses 1 to 26, wherein said rotavirus is rotavirus C.

28. The immunogenic composition of clause 1 to 27, wherein the immunogenic fragment of a rotavirus VP8 protein consists of an amino acid sequence having at least 90%, preferably at least 95%, more preferably at least 98% or still more preferably at least 99% sequence identity with the sequence of SEQ ID NO:6.

29. The immunogenic composition of any one of clauses 1 to 28, wherein said immunogenic fragment of a rotavirus VP8 protein consists of or is
an immunogenic fragment of a rotavirus A VP8 protein, as specified in any one or more of clauses 9 to 24, or
a consensus sequence of a portion of a rotavirus VP8 protein, in particular of a portion of a rotavirus A VP8 protein, as specified in any one of clauses 9 to 13, 25 and 26, or
an immunogenic fragment of a rotavirus C VP8 protein, as specified in any one of clauses 9 to 12, 27 and 28.

30. The immunogenic composition of any one of clauses 1 to 29, wherein the immunogenic fragment of a rotavirus VP8 protein consists of an amino acid sequence having at least 90%, preferably at least 95%, more preferably at least 98% or still more preferably at least 99% sequence identity with a sequence selected from the group consisting of SEQ ID NO:3, SEQ ID NO:4, SEQ ID NO:5 and SEQ ID NO:6.

31. The immunogenic composition of any one of clauses 1 to 30,
wherein said immunoglobulin Fc fragment is at least 220 amino acid residues in length, preferably 220 to 250 amino acid residues in length,
and/or wherein the immunoglobulin Fc fragment is non-glycosylated.

32. The immunogenic composition of any one of clauses 1 to 31, wherein said immunoglobulin Fc fragment comprises or consists of the heavy-chain constant region 2 (CH2) and the heavy-chain constant region 3 (CH3), and optionally the hinge region or a part of the hinge region, of an immunoglobulin.

33. The immunogenic composition of any one of clauses 1 to 32, wherein said immunoglobulin is selected from the group consisting of IgG, IgA, IgD, IgE and IgM.

34. The immunogenic composition of any one of clauses 1 to 33, wherein said immunoglobulin Fc fragment is an immunoglobulin Fc fragment encoded by the genome of a species whose intestinal cells are susceptible to an infection by the rotavirus from which the immunogenic fragment of a rotavirus VP8 protein is derived.

35. The immunogenic composition of any one of clauses 1 to 34, wherein said immunoglobulin Fc fragment is a swine IgG Fc fragment.

36. The immunogenic composition of any one of clauses 1 to 35, wherein said immunoglobulin Fc fragment comprises or consists of an amino acid sequence having at least 70%, preferably at least 80%, more preferably at least 90%, still more preferably at least 95% or in particular 100% sequence identity with a sequence selected from the group consisting of SEQ ID NO:7 and SEQ ID NO:8.

37. The immunogenic composition of any one of clauses 3 to 36, wherein said linker moiety is an amino acid sequence being 1 to 50 amino acid residues in length.

38. The immunogenic composition of any one of clauses 3 to 37, wherein said linker moiety comprises or consists of an amino acid sequence having at least 66%, preferably at least 80%, more preferably at least 90%, still more preferably at least 95% or in particular 100% sequence identity with a sequence selected from the group consisting of SEQ ID NO:9 (Gly-Gly-Ser), SEQ ID NO:10 and SEQ ID NO:11.

39. The immunogenic composition of any one of clauses 5 to 38, wherein said polypeptide has an N-terminal methionine residue flanking the N-terminal amino acid residue of said immunogenic fragment of a rotavirus VP8 protein.

40. The immunogenic composition of any one of clauses 5 to 39, wherein said polypeptide comprises a further immunogenic fragment of a rotavirus VP8 protein linked to the C-terminus of said immunoglobulin Fc fragment.

41. The immunogenic composition of any one of clauses 1 to 40, wherein said polypeptide comprises
an immunogenic fragment (1) of a rotavirus VP8 protein,
an immunoglobulin Fc fragment, and
a further immunogenic fragment (2) of a rotavirus VP8 protein,
wherein said immunoglobulin Fc fragment is linked to the C-terminus of said immunogenic fragment (1),
and wherein said further immunogenic fragment (2) of a rotavirus VP8 protein is linked to the C-terminus of said immunoglobulin Fc fragment.

42. The immunogenic composition of clause 40 or 41, wherein said further immunogenic fragment of a rotavirus VP8 protein consists of or is
an immunogenic fragment of a rotavirus A VP8 protein, as specified in any one or more of clauses 9 to 24; or
a consensus sequence of a portion of a rotavirus VP8 protein, in particular of a portion of a rotavirus A VP8 protein, as specified in any one or more of clauses 9 to 13, 25 and 26; or
an immunogenic fragment of a rotavirus C VP8 protein, as specified in any one or more of clauses 9 to 12, 27 and 28.

43. The immunogenic composition of any one of clauses 40 to 42, wherein said further immunogenic fragment of a rotavirus VP8 protein comprises or consists of an amino acid sequence having at least 90%, preferably at least 95%, more preferably at least 98% or still more preferably at least 99% sequence identity with a sequence selected from the group consisting of SEQ ID NOs: 2 to 6,
and/or wherein said further immunogenic fragment of a rotavirus VP8 protein is different from the immunogenic fragment of a rotavirus VP8 protein of which the C-terminus is linked to said immunoglobulin Fc fragment.

44. The immunogenic composition of any one of clauses 40 to 43,
wherein said further immunogenic fragment of a rotavirus VP8 protein is linked to the C-terminus of said immunoglobulin Fc fragment via a linker moiety, wherein said linker moiety is preferably a linker moiety as specified in clause 37 or 38,
or wherein said further immunogenic fragment of a rotavirus VP8 protein is linked to the C-terminus of said immunoglobulin Fc fragment via a peptide bond between the N-terminal amino acid residue of said further immunogenic fragment of a rotavirus VP8 protein and the C-terminal amino acid residue of said immunoglobulin Fc fragment.

45. The immunogenic composition of any one of clauses 1 to 44, wherein said polypeptide consists of:
an immunogenic fragment of a rotavirus VP8 protein, in particular an immunogenic fragment of a rotavirus VP8 protein as specified in any one or more of clauses 9 to 30,
an N-terminal methionine residue flanking the N-terminal amino acid residue of said immunogenic fragment of a rotavirus VP8 protein, and
an immunoglobulin Fc fragment, in particular an immunoglobulin Fc fragment as specified in any one or more of clauses 31 to 36,
wherein said immunoglobulin Fc fragment is linked to the C-terminus of said immunogenic fragment of a rotavirus VP8 protein, in particular via a linker moiety, wherein said linker moiety is preferably a linker moiety as specified in clause 37 or 38,
and optionally a further immunogenic fragment of a rotavirus VP8 protein linked to the C-terminus of said immunoglobulin Fc fragment, in particular via a linker moiety, wherein said further immunogenic fragment of a rotavirus VP8 protein is preferably the further immunogenic fragment as specified in any one or more of clauses 41 to 44, and wherein said linker moiety is preferably a linker moiety as specified in clause 37 or 38.

46. The immunogenic composition of any one of clauses 1 to 45, wherein said polypeptide is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12, SEQ ID NO:13, SEQ ID NO:14, SEQ ID NO:15 and SEQ ID NO:16.

47. The immunogenic composition of any one of clauses 1 to 46, wherein said polypeptide is a recombinant protein, in particular a recombinant baculovirus expressed protein.

48. The immunogenic composition of any one of clauses 1 to 47, wherein said polypeptide forms a homodimer with a further identical polypeptide.

49. The immunogenic composition of any one of clauses 1 to 48, wherein component (i) is present in a multimer comprising or composed of a plurality of said polypeptide, and wherein said multimer is preferably a homodimer formed by said polypeptide with a further identical polypeptide.

50. The immunogenic composition of any one of clauses 1 to 49, wherein the at least one immunogenic substance, different from said polypeptide, consists of two or more immunogenic substances, wherein all of said immunogenic substances are different from said polypeptide and different from each other.

51. The immunogenic composition of any one of clauses 1 to 50, wherein the at least one immunogenic substance, different from said polypeptide, is at least one immunogenic substance which comprises a reovirus antigen, different from said immunogenic fragment.

52. The immunogenic composition of any one of clauses 1 to 51, wherein the at least one immunogenic substance, different from said polypeptide, consists of two or more immunogenic substances, wherein each of said substances comprises a reovirus antigen, and wherein all of said reovirus antigens are different from said immunogenic fragment and different from each other.

53. The immunogenic composition of any one of clauses 1 to 52, wherein the at least one immunogenic substance, different from said polypeptide, is at least a protein which comprises a reovirus antigen, different from said immunogenic fragment.

54. The immunogenic composition of any one of clauses 1 to 53, wherein the at least one immunogenic substance, different from said polypeptide, consists of two or more proteins, wherein each of said proteins comprises a reovirus antigen, and wherein all of said reovirus antigens are different from said immunogenic fragment and different from each other.

55. The immunogenic composition of any one of clauses 1 to 54, wherein component (ii) consists of at least one immunogenic substance which comprises a rotavirus antigen, different from said immunogenic fragment of component (i).

56. The immunogenic composition of any one of clauses 1 to 55, wherein component (ii) consists of two or more immunogenic substances, wherein each of said substances comprises a rotavirus antigen, and wherein all of said rotavirus antigens are different from said immunogenic fragment of component (i) and different from each other.

57. The immunogenic composition of any one of clauses 1 to 56, wherein component (ii) consists of at least one protein which comprises a rotavirus antigen, different from said immunogenic fragment of component (i).

58. The immunogenic composition of any one of clauses 1 to 57, wherein component (ii) consists of two or more proteins, wherein each of said proteins comprises a rotavirus antigen, and wherein all of said rotavirus antigens are different from said immunogenic fragment of component (i) and different from each other.

59. The immunogenic composition of one or more of clauses 1 to 58, wherein
said polypeptide is a first polypeptide;
and
the at least one immunogenic substance, different from said polypeptide, is at least one additional polypeptide, different from said first polypeptide.

60. The immunogenic composition of clause 59, wherein the at least one immunogenic substance, different from said polypeptide, comprises or is
a second polypeptide different from said first polypeptide,
and preferably comprises a third polypeptide, different from both said first and second polypeptide, and
optionally comprises a fourth polypeptide, different from all of said first to third polypeptides.

61. The immunogenic composition of clause 59 or 60, wherein the at least one immunogenic substance, different from said polypeptide, comprises or is
a second polypeptide which comprises a reovirus antigen, different from said immunogenic fragment,
and preferably comprises a third polypeptide, different from both said first and second polypeptide, and
optionally comprises a fourth polypeptide, different from all of said first to third polypeptides.

62. The immunogenic composition of clause 61, wherein said third polypeptide comprises a reovirus antigen, different from both said immunogenic fragment and reovirus antigen of the second polypeptide,
and wherein optionally said fourth polypeptide comprises a reovirus antigen, different from all of
   said immunogenic fragment,
   the reovirus antigen of the second polypeptide and
   the reovirus antigen of the third polypeptide.

63. The immunogenic composition of one or more of clauses 51 to 54, and 61 to 62, wherein said reovirus antigen is a rotavirus antigen and, respectively, said reovirus antigens are rotavirus antigens.

64. The immunogenic composition of one or more of clauses 60 to 63,
wherein said second polypeptide is a polypeptide as specified in any one of clauses 1 to 49, and wherein said second peptide is different from said first polypeptide,
and wherein preferably said third polypeptide is a polypeptide as specified in any one of clauses 1 to 49, and wherein said third polypeptide is different from both said first and second polypeptide,
and wherein optionally said fourth polypeptide is a polypeptide as specified in any one of clauses 1 to 49, and wherein said fourth polypeptide is different from all of said first to third polypeptides.

65. The immunogenic composition of one or more of clauses 1 to 64, wherein
said polypeptide is a first polypeptide comprising
   a first immunogenic fragment of a rotavirus VP8 protein, and
   an immunoglobulin Fc fragment;
and
the at least one immunogenic substance, different from said polypeptide, comprises or consists of
a second polypeptide comprising a second immunogenic fragment of a rotavirus VP8 protein, wherein said second immunogenic fragment is different from said first immunogenic fragment,
and preferably a third polypeptide comprising a third immunogenic fragment of a rotavirus VP8 protein, wherein said third immunogenic fragment is different from both said first and second immunogenic fragment,
and optionally a fourth polypeptide comprising a fourth immunogenic fragment of a rotavirus VP8 protein, wherein said fourth immunogenic fragment is different from all of said first to third immunogenic fragments.

66. The immunogenic composition of clause 65, wherein each of said second to fourth immunogenic fragments is individually selected from the group consisting of
an immunogenic fragment as specified in any one or more of clauses 9 to 24,
a consensus sequence of a portion of a rotavirus VP8 protein, in particular of a portion of a rotavirus A VP8 protein, as specified in any one or more of clauses 9 to 13, 25 and 26,
and
an immunogenic fragment of a rotavirus C VP8 protein, as specified in any one or more of clauses 9 to 12, 27 and 28,
in particular provided that all of said second to fourth immunogenic fragments are different from said first immunogenic fragment and different from each other.

67. The immunogenic composition of clause 65 or 66, wherein
said first immunogenic fragment is selected from the group consisting of X7, X13, X6 and XC, and/or
said second immunogenic fragment is selected from the group consisting of X7, X13, X6 and XC, and/or
said third immunogenic fragment is selected from the group consisting of X7, X13, X6 and XC, and/or
said fourth immunogenic fragments is selected from the group consisting of X7, X13, X6 and XC;
wherein
X7 represents an immunogenic fragment of a genotype P[7] rotavirus VP8 protein,
X13 represents an immunogenic fragment of a genotype P[13] rotavirus VP8 protein,
X6 represents an immunogenic fragment of a genotype P[6] rotavirus VP8 protein, and
XC represents an immunogenic fragment of a rotavirus C VP8 protein.

68. The immunogenic composition of clause 67, wherein the at least one immunogenic substance, different from said polypeptide, comprises or is
a second polypeptide comprising a second immunogenic fragment of a rotavirus VP8 protein,
and wherein each of said first and second immunogenic fragment is individually selected from the group consisting of X7, X13, X6 and XC,
provided that when said first immunogenic fragment is X7, then said second immunogenic fragment is selected from the group consisting of X13, X6 and XC,
provided that when said first immunogenic fragment is X6, then said second immunogenic fragment is selected from the group consisting of X7, X13 and XC,
provided that when said first immunogenic fragment is X13, then said second immunogenic fragment is selected from the group consisting of X7, X6 and XC,
and provided that when said first immunogenic fragment is XC, then said second immunogenic fragment is selected from the group consisting of X7, X13 and X6.

69. The immunogenic composition of clause 67 or 68, wherein the at least one immunogenic substance, different from said polypeptide, comprises or consists of
a second polypeptide comprising a second immunogenic fragment of a rotavirus VP8 protein, and
a third polypeptide comprising a third immunogenic fragment of a rotavirus VP8 protein, wherein each of said first to third immunogenic fragments is individually selected from the group consisting of X7, X13, X6 and XC,
provided that when said first immunogenic fragment is X7, then said second immunogenic fragment is X13 and said third immunogenic fragment is selected from the group consisting of X6 and XC,
provided that when said first immunogenic fragment is X6, then said second immunogenic fragment is X7 and said third immunogenic fragment is selected from the group consisting of X13 and XC,
provided that when said first immunogenic fragment is X13, then said second immunogenic fragment is X7 and said third immunogenic fragment is selected from the group consisting of X6 and XC,
and provided that when said first immunogenic fragment is XC, then said second immunogenic fragment is X7 and said third immunogenic fragment is selected from the group consisting of X13 and X6.

70. The immunogenic composition of one or more of clauses 67 to 69, wherein the at least one immunogenic substance, different from said polypeptide, comprises or consists of
a second polypeptide comprising a second immunogenic fragment of a rotavirus VP8 protein, and
a third polypeptide comprising a third immunogenic fragment of a rotavirus VP8 protein, and
a fourth polypeptide comprising a fourth immunogenic fragment of a rotavirus VP8 protein,
and wherein each of said first to fourth immunogenic fragments is individually selected from the group consisting of X7, X13, X6 and XC,
provided that when said first immunogenic fragment is X7, then said second immunogenic fragment is X13, said third immunogenic fragment is X6 and said fourth immunogenic fragment is XC,
provided that when said first immunogenic fragment is X6, then said second immunogenic fragment is X7, said third immunogenic fragment is X13 and said fourth immunogenic fragment is XC,
provided that when said first immunogenic fragment is X13, then said second immunogenic fragment is X7, said third immunogenic fragment is X6 and said fourth immunogenic fragment is XC,
and provided that when said first immunogenic fragment is XC, then said second immunogenic fragment is X7, said third immunogenic fragment is X13 and said fourth immunogenic fragment is X6.

71. The immunogenic composition of one or more clauses 67 to 70, wherein
X7 consists of an amino acid sequence having at least 90%, preferably at least 95%, more preferably at least 98% or still more preferably at least 99% sequence identity with the sequence of SEQ ID NO:3, and/or
X13 consists of an amino acid sequence having at least 90%, preferably at least 95%, more preferably at least 98% or still more preferably at least 99% sequence identity with the sequence of SEQ ID NO:5, and/or
X6 consists of an amino acid sequence having at least 90%, preferably at least 95%, more preferably at least 98% or still more preferably at least 99% sequence identity with the sequence of SEQ ID NO:4, and/or
XC consists of an amino acid sequence having at least 90%, preferably at least 95%, more preferably at least 98% or still more preferably at least 99% sequence identity with the sequence of SEQ ID NO:6.

72. The immunogenic composition of one or more of clauses 65 to 71, wherein said first immunogenic fragment is an immunogenic fragment of a genotype P[7] rotavirus VP8 protein, and
said second immunogenic fragment is an immunogenic fragment of a genotype P[13] rotavirus VP8 protein,
and preferably said third immunogenic fragment is an immunogenic fragment of a genotype P[6] rotavirus VP8 protein, and
and optionally said fourth immunogenic fragment is an immunogenic fragment of a rotavirus C VP8 protein.

73. The immunogenic composition of one or more of clauses 65 to 72, wherein
said first immunogenic fragment consists of an amino acid sequence having at least 90%, preferably at least 95%, more preferably at least 98% or still more preferably at least 99% sequence identity with the sequence of SEQ ID NO:3, and
said second immunogenic fragment consists of an amino acid sequence having at least 90%, preferably at least 95%, more preferably at least 98% or still more preferably at least 99% sequence identity with the sequence of SEQ ID NO:5,
and preferably said third immunogenic fragment consists of an amino acid sequence having at least 90%, preferably at least 95%, more preferably at least 98% or still more preferably at least 99% sequence identity with the sequence of SEQ ID NO:4, and
and optionally said fourth immunogenic fragment consists of an amino acid sequence having at least 90%, preferably at least 95%, more preferably at least 98% or still more preferably at least 99% sequence identity with the sequence of SEQ ID NO:6.

74. The immunogenic composition of one or more of clauses 59 to 73, wherein
said first polypeptide is selected from the group consisting of R7, R13, R6 and RC, and/or
said second polypeptide is selected from the group consisting R7, R13, R6 and RC, and/or
said third polypeptide is selected from the group consisting of R7, R13, R6 and RC, and/or
said fourth polypeptides is selected from the group consisting of R7, R13, R6 and RC;
wherein
R7 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12,
R13 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14,
R6 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:13, and
RC is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:15.

75. The immunogenic composition of one or more of clauses 59 to 74, wherein
the at least one immunogenic substance, different from said polypeptide, comprises or is a second polypeptide of a rotavirus VP8 protein, and wherein
each of said first and second polypeptide is individually selected from the group consisting of R7, R13, R6 and RC,
provided that when said first polypeptide is R7, then said second polypeptide is selected from the group consisting of R13, R6 and RC,
provided that when said first polypeptide is R6, then said second polypeptide is selected from the group consisting of R7, R13 and RC,
provided that when said first polypeptide is R13, then said second polypeptide is selected from the group consisting of R7, R6 and RC,
and provided that when said first polypeptide is RC, then said second polypeptide is selected from the group consisting of R7, R13 and R6.

76. The immunogenic composition of clause 74 or 75, wherein
the at least one immunogenic substance, different from said polypeptide, comprises or consists of a second polypeptide of a rotavirus VP8 protein, and wherein
each of said first and second polypeptide is individually selected from the group consisting of R7, R13 and R6,
provided that when said first polypeptide is R7, then said second polypeptide is selected from the group consisting of R13 and R6,
provided that when said first polypeptide is R6, then said second polypeptide is selected from the group consisting of R7 and R13,
and provided that when said first polypeptide is R13, then said second polypeptide is selected from the group consisting of R7 and R6.

77. The immunogenic composition of one or more of clauses 74 to 76, wherein
said first polypeptide is R7 and said second polypeptide is selected from the group consisting of R13 and R6.

78. The immunogenic composition of one or more of clauses 74 to 77, wherein
the at least one immunogenic substance, different from said polypeptide, comprises or consists of
a second polypeptide, and
a third polypeptide,
and wherein each of said first to third polypeptides is individually selected from the group consisting of R7, R13, R6 and RC,
provided that when said first polypeptide is R7, then said second polypeptide is R13 and said third polypeptide is selected from the group consisting of R6 and RC,
provided that when said first polypeptide is R6, then said second polypeptide is R7 and said third polypeptide is selected from the group consisting of R13 and RC,
provided that when said first polypeptide is R13, then said second polypeptide is R7 and said third polypeptide is selected from the group consisting of R6 and RC,
and provided that when said first polypeptide is RC, then said second polypeptide is R7 and said third polypeptide is selected from the group consisting of R13 and R6.

79. The immunogenic composition of one or more of clauses 74 to 78, wherein
said first polypeptide is R7, and
said second polypeptide is R13, and
said third polypeptide is selected from the group consisting of R6 and RC.

80. The immunogenic composition of one or more of clauses 74 to 79, wherein
the at least one immunogenic substance, different from said polypeptide, comprises or consists of
a second polypeptide, and
a third polypeptide,
and wherein each of said first to third polypeptides is individually selected from the group consisting of R7, R13 and R6,
provided that when said first polypeptide is R7, then said second polypeptide is R13 and said third polypeptide is R6,
provided that when said first polypeptide is R6, then said second polypeptide is R7 and said third polypeptide is R13,
provided that when said first polypeptide is R13, then said second polypeptide is R7 and said third polypeptide is R6.

81. The immunogenic composition of one or more of clauses 74 to 80, wherein
the at least one immunogenic substance, different from said polypeptide, comprises or consists of
a second polypeptide, and
a third polypeptide, and
a fourth polypeptide,
and wherein each of said first to fourth polypeptides is individually selected from the group consisting of R7, R6, R13 and RC,
provided that when said first polypeptide is R7, then said second polypeptide is R13, said third polypeptide is R6 and said fourth polypeptide is RC,
provided that when said first polypeptide is R6, then said second polypeptide is R7, said third polypeptide is R13 and said fourth polypeptide is RC,
provided that when said first polypeptide is R13, then said second polypeptide is R7, said third polypeptide is R6 and said fourth polypeptide is RC,
and provided that when said first polypeptide is RC, then said second polypeptide is R7, said third polypeptide is R13 and said fourth polypeptide is R6

82. The immunogenic composition of one or more of clauses 74 to 81, wherein
said first polypeptide is R7, and
said second polypeptide is R13, and
said third polypeptide is R6, and
said fourth polypeptide is RC.

83. An immunogenic composition, in particular the immunogenic composition of any one of clauses 1 to 82, which comprises
a protein comprising or consisting of an amino acid sequence having at least 70%, preferably at least 80%, more preferably at least 90%, still more preferably at least 95% or in particular 100% sequence identity with the sequence of SEQ ID NO:12, and
a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, SEQ ID NO:13 and SEQ ID NO:15.

84. An immunogenic composition, in particular the immunogenic composition of any one of clauses 1 to 83, which comprises
a protein comprising or consisting of an amino acid sequence having at least 70%, preferably at least 80%, more preferably at least 90%, still more preferably at least 95% or in particular 100% sequence identity with the sequence of SEQ ID NO:12, and
a protein comprising or consisting of an amino acid sequence having at least 70%, preferably at least 80%, more preferably at least 90%, still more preferably at least 95% or in particular 100% sequence identity with the sequence of SEQ ID NO:14.

85. The immunogenic composition of clause 84, which comprises
a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:13.

86. The immunogenic composition of clause 84 or 85, which comprises
a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:15.

87. The immunogenic composition of any one of clauses 1 to 86, wherein the immunogenic composition further comprises a pharmaceutical- or veterinary-acceptable carrier or excipient.

88. The immunogenic composition of any one of clauses 1 to 87, wherein the immunogenic composition further comprises an adjuvant.

89. The immunogenic composition of any one of clauses 1 to 88 comprising
a pharmaceutical- or veterinary-acceptable carrier or excipient,
and optionally an adjuvant.

90. The immunogenic composition of clause 88 or 89, wherein the adjuvant is an emulsified oil-in-water adjuvant.

91. The immunogenic composition of clause 88 or 89, wherein the adjuvant is a carbomer.

92. Use of the immunogenic composition of any one of clauses 1 to 91 for the preparation of a medicament, preferably of a vaccine.

93. The immunogenic composition of any one of clauses 1 to 91 for use as a medicament.

94. The immunogenic composition of any one of clauses 1 to 91 for use as a vaccine.

95. The immunogenic composition of any one of clauses 1 to 91 for use in a method for inducing an immune response against rotavirus in a subject.

96. The immunogenic composition of any one of clauses 1 to 91 for use in a method of reducing or preventing one or more clinical signs, mortality or fecal shedding caused by a rotavirus infection in a subject or for use in a method of treating or preventing an infection with rotavirus in a subject.

97. The immunogenic composition according to clause 95 or 96, wherein the subject is a mammal or a bird, and wherein the bird is preferably a chicken.

98. The immunogenic composition according to any one of clauses 95 to 97, wherein the subject is a mammal, and wherein the mammal is preferably a swine or a bovine.

99. The immunogenic composition according to any one of clauses 95 to 98, wherein the subject is a pig, and wherein the pig is preferably a piglet or a sow.

100. The immunogenic composition according to clause 95, wherein the subject is a pregnant sow.

101. The immunogenic composition according to clause 96, wherein the subject is a piglet.

102. The immunogenic composition of any one of clauses 1 to 91 for use in a method of reducing or preventing one or more clinical signs, mortality or fecal shedding caused by a rotavirus infection in a piglet, wherein the piglet is to be suckled by a sow to which the immunogenic composition has been administered.

103. The immunogenic composition according to clause 102, wherein said sow to which the immunogenic composition has been administered is a sow to which the immunogenic composition has been administered while said sow has been pregnant, in particular with said piglet.

104. A method for the treatment or prevention of a rotavirus infection, the reduction, prevention or treatment of one or more clinical signs, mortality or fecal shedding caused by a rotavirus infection, or the prevention or treatment of a disease caused by a rotavirus infection, comprising administering the immunogenic composition of any one of clauses 1 to 91 to a subject.

105. A method for inducing the production of antibodies specific for rotavirus in a sow, wherein said method comprises administering the immunogenic composition of any one of clauses 1 to 91 to said sow.

106. A method of reducing or preventing one or more clinical signs, mortality or fecal shedding caused by a rotavirus infection in a piglet, wherein said method comprises
administering the immunogenic composition of any one of clauses 1 to 91 to a sow, and
allowing said piglet to be suckled by said sow.

107. The method of clause 106, wherein said sow is a sow being pregnant, in particular with said piglet.

108. The method of clause 106 or 107, comprising the steps of
administering the immunogenic composition of any one of clauses 1 to 91 to a sow being pregnant with said piglet,
allowing said sow to give birth to said piglet, and
allowing said piglet to be suckled by said sow.

109. A method of reducing one or more clinical signs, mortality or fecal shedding caused by a rotavirus infection in a piglet, wherein the piglet is to be suckled by a sow to which the immunogenic composition of any one of clauses 1 to 91 has been administered.

110. The immunogenic composition according to any one of clauses 96 to 103 or the method of any one of clauses 104 to 109, wherein said one or more clinical signs are selected from the group consisting of
diarrhea,
rotavirus colonization,
lesions, in particular macroscopic lesions,
decreased average daily weight gain, and
gastroenteritis.

111. The immunogenic composition according to clause 110 or the method of clause 110, wherein said rotavirus colonization is a rotavirus colonization of the intestine and/or wherein said lesions are enteric lesions.

112. The immunogenic composition according to any one of clauses 95 to 103, 110 and 111, or the method of any one of clauses 104 to 111, wherein
said rotavirus infection is an infection with genotype P[23] rotavirus and/or genotype P[7] rotavirus,
said infection with rotavirus is an infection with a genotype P[23] rotavirus and/or genotype P[7] rotavirus,
said immune response against rotavirus is an immune response against genotype P[23] rotavirus and/or genotype P[7] rotavirus, or
said antibodies specific for rotavirus are antibodies specific for genotype P[23] rotavirus and/or genotype P[7] rotavirus.

113. The immunogenic composition or the method according to clause 112, wherein the immunogenic composition comprises a polypeptide as specified in any one of clauses 21 to 26 and 29 to 48, wherein said immunogenic fragment of a rotavirus VP8 protein is an immunogenic fragment of a genotype P[7] rotavirus VP8 protein.

114. The immunogenic composition according to clause 113, wherein said immunogenic fragment of a genotype P[7] rotavirus VP8 protein consists of an amino acid sequence having at least 90%, preferably at least 95%, more preferably at least 98% or still more preferably at least 99% sequence identity with the sequence of SEQ ID NO:3.

115. A method of producing the immunogenic composition of any one of clauses 1 to 91, wherein the method comprises the steps of:
(a) permitting infection of susceptible cells in culture with a vector comprising a nucleic acid sequence encoding a polypeptide as specified in any one of clauses 1 to 48, wherein said polypeptide is expressed by said vector;
(b) thereafter recovering said polypeptide, in particular in the cell culture supernatant, wherein preferably cell debris is separated from said polypeptide via a separation step, preferably including a micro filtration through at least one filter, preferably two filters, wherein the at least one filter preferably has a pore size of about 1 to about 20 μm and/or about 0.1 μm to about 4 μm;
(c) inactivating the vector by adding binary ethylenimine (BEI) to the mixture of step (b);
(d) neutralizing the BEI by adding sodium thiosulfate to the mixture resulting from step (c); and
(e) concentrating the polypeptide in the mixture resulting from step (d) by removing a portion of the liquid from the mixture by a filtration step utilizing a filter with a filter membrane having a molecular weight cut off of between about 5 kDa and about 100 kDa, preferably between about 10 kDa and about 50 kDa;
and wherein said method comprises the steps of
(f) admixing the mixture remaining after step (e) with at least one immunogenic substance, different from said polypeptide;
(g) and optionally admixing the mixture remaining after step (f) with a further component selected from the group consisting of pharmaceutically acceptable carriers, adjuvants, diluents, excipients, and combinations thereof, or wherein said method comprises the steps of
(f) admixing the mixture remaining after step (e) with a further component selected from the group consisting of pharmaceutically acceptable carriers, adjuvants, diluents, excipients, and combinations thereof, and
(g) admixing the mixture remaining after step (f) with at least one immunogenic substance, different from said polypeptide.

116. The method of clause 115, wherein said at least one immunogenic substance is the at least one immunogenic substance as specified in any one of clauses 50 to 76.

117. The method of clause 115 or 116, wherein in step (a) the polypeptide as specified in any one of clauses 1 to 48 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12.

118. The method of any one of clauses 115 to 117, wherein said at least one immunogenic substance, different from said polypeptide, is at least one protein selected from the group consisting of
a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14,
a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:13, and
a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:15.

119. The immunogenic composition according to any one of clauses 1 to 91, 93 to 103 and 110 to 114, the use of clause 92, or the method of any one of clauses 104 to 112, 113 and 114, wherein the immunogenic composition is obtainable by the method of any one of clauses 115 to 118.

1. An immunogenic composition which comprises
(i) a polypeptide comprising
an immunogenic fragment of a rotavirus VP8 protein, and
an immunoglobulin Fc fragment,
and
(ii) at least one immunogenic substance, different from said polypeptide.

2. The immunogenic composition of claim 1, wherein said immunoglobulin Fc fragment is linked to the C-terminus of said immunogenic fragment of a rotavirus VP8 protein via a linker moiety,
or wherein said immunoglobulin Fc fragment is linked to the C-terminus of said immunogenic fragment of a rotavirus VP8 protein via a peptide bond between the N-terminal amino acid residue of said immunoglobulin Fc fragment and the C-terminal amino acid residue of said immunogenic fragment of a rotavirus VP8 protein.

3. The immunogenic composition of claim 1 or 2, wherein said polypeptide is a fusion protein of the formula x-y-z, wherein
x consists of an immunogenic fragment of a rotavirus VP8 protein;
y is a linker moiety; and
z is an immunoglobulin Fc fragment.

4. The immunogenic composition of any one of claims 1 to 3, wherein said rotavirus is porcine rotavirus,
and/or wherein said rotavirus is selected from the group consisting of rotavirus A and rotavirus C.

5. The immunogenic composition of any one of claims 1 to 4, wherein said immunogenic fragment of a rotavirus VP8 protein is an N-terminally extended lectin-like domain of a rotavirus VP8 protein, wherein the N-terminal extension is 1 to 20 amino acid residues, preferably 5 to 15 amino acid residues, in length.

6. The immunogenic composition of any one of claims 1 to 5, wherein said rotavirus is selected from the group consisting of genotype P[7] rotavirus, genotype P[6] rotavirus and genotype P[13] rotavirus.

7. The immunogenic composition of any one of claims 1 to 6, wherein the immunogenic fragment of a rotavirus VP8 protein consists of or is a consensus sequence of a portion of a rotavirus VP8 protein, in particular of a portion of a rotavirus A VP8 protein, and wherein said consensus sequence of a portion of a rotavirus VP8 protein is preferably obtainable by a method comprising the steps of:
translating a plurality of nucleotide sequences encoding a portion of a rotavirus VP8 protein into amino acid sequences,
aligning said amino acid sequences to known rotavirus VP8 proteins, preferably by using MUSCLE sequence alignment software UPGMB clustering and default gap penalty parameters,
subjecting said aligned sequences to a phylogenetic analysis and generating a neighbor joining phylogeny reconstruction based on rotavirus VP8 protein sequence, in particular importing said aligned amino acid sequences into MEGA7 software for phylogenetic analysis and generating a neighbor joining phylogeny reconstruction based on rotavirus VP8 protein sequence,
computing the optimal tree using the Poisson correction method with bootstrap test of phylogeny (n=100),
drawing the optimal tree to scale with branch lengths equal to evolutionary distances in units of amino acid substitutions per site over 170 total positions,
considering nodes where bootstrap cluster association is greater than 70% as significant,
designating nodes with approximately 10% distance and bootstrap cluster associations greater than 70% as clusters, and
selecting a cluster and generating the consensus sequences by identifying the greatest frequency per aligned position within the cluster,
and optionally, in cases where equivalent proportions of amino acids are observed in an aligned position, selecting the amino acid residue based on reported epidemiological data in conjunction with a predefined product protection profile.

8. The immunogenic composition of any one of claims 1 to 7, wherein the immunogenic fragment of a rotavirus VP8 protein consists of an amino acid sequence having at least 90%, preferably at least 95%, more preferably at least 98% or still more preferably at least 99% sequence identity with a sequence selected from the group consisting of SEQ ID NO:3, SEQ ID NO:4, SEQ ID NO:5 and SEQ ID NO:6.

9. The immunogenic composition of any one of claims 1 to 8, wherein said immunoglobulin Fc fragment is an immunoglobulin Fc fragment encoded by the genome of a species whose intestinal cells are susceptible to an infection by the rotavirus from which the immunogenic fragment of a rotavirus VP8 protein is derived,
and/or wherein said immunoglobulin Fc fragment is preferably a swine IgG Fc fragment,
and/or wherein said immunoglobulin Fc fragment comprises or consists of an amino acid sequence having at least 70%, preferably at least 80%, more preferably at least 90%, still more preferably at least 95% or in particular 100% sequence identity with a sequence selected from the group consisting of SEQ ID NO:7 and SEQ ID NO:8.

10. The immunogenic composition of any one of claims 1 to 9, wherein said linker moiety is an amino acid sequence being 1 to 50 amino acid residues in length,
and/or wherein said linker moiety comprises or consists of an amino acid sequence having at least 66%, preferably at least 80%, more preferably at least 90%, still more preferably at least 95% or in particular 100% sequence identity with a sequence selected from the group consisting of SEQ ID NO:9 (Gly-Gly-Ser), SEQ ID NO:10 and SEQ ID NO:11.

11. The immunogenic composition of any one of claims 1 to 10, wherein said polypeptide is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12, SEQ ID NO:13, SEQ ID NO:14, SEQ ID NO:15 and SEQ ID NO:16.

12. The immunogenic composition of any one of claims 1 to 11, wherein the at least one immunogenic substance, different from said polypeptide, consists of two or more immunogenic substances, wherein all of said immunogenic substances are
different from said polypeptide and
different from each other.

13. The immunogenic composition of any one of claims 1 to 12, wherein component (ii) consists of two or more immunogenic substances, wherein each of said substances comprises a rotavirus antigen, and wherein all of said rotavirus antigens are different from said immunogenic fragment of component (i) and different from each other.

14. The immunogenic composition of any one of claims 1 to 13, wherein
said polypeptide is a first polypeptide comprising
a first immunogenic fragment of a rotavirus VP8 protein, and
an immunoglobulin Fc fragment;
and
the at least one immunogenic substance, different from said polypeptide, comprises or consists of
a second polypeptide comprising a second immunogenic fragment of a rotavirus VP8 protein, wherein said second immunogenic fragment is different from said first immunogenic fragment,
and preferably a third polypeptide comprising a third immunogenic fragment of a rotavirus VP8 protein, wherein said third immunogenic fragment is different from both said first and second immunogenic fragment,
and optionally a fourth polypeptide comprising a fourth immunogenic fragment of a rotavirus VP8 protein, wherein said fourth immunogenic fragment is different from all of said first to third immunogenic fragments.

15. The immunogenic composition of claim 14, wherein the at least one immunogenic substance, different from said polypeptide, comprises or consists of
a second polypeptide comprising a second immunogenic fragment of a rotavirus VP8 protein, and
a third polypeptide comprising a third immunogenic fragment of a rotavirus VP8 protein,
and wherein each of said first to third immunogenic fragments is individually selected from the group consisting of X7, X6, X13 and XC,
wherein
X7 represents an immunogenic fragment of a genotype P[7] rotavirus VP8 protein,
X6 represents an immunogenic fragment of a genotype P[6] rotavirus VP8 protein,
X13 represents an immunogenic fragment of a genotype P[13] rotavirus VP8 protein, and
XC represents an immunogenic fragment of a rotavirus C VP8 protein;
provided that when said first immunogenic fragment is X7, then said second immunogenic fragment is X6 and said third immunogenic fragment is selected from the group consisting of X13 and XC,
provided that when said first immunogenic fragment is X6, then said second immunogenic fragment is X7 and said third immunogenic fragment is selected from the group consisting of X13 and XC,
provided that when said first immunogenic fragment is X13, then said second immunogenic fragment is X7 and said third immunogenic fragment is selected from the group consisting of X6 and XC,
and provided that when said first immunogenic fragment is XC, then said second immunogenic fragment is X7 and said third immunogenic fragment is selected from the group consisting of X6 and X13.

16. The immunogenic composition of claim 14 or 15, wherein the at least one immunogenic substance, different from said polypeptide, comprises or consists of
a second polypeptide, and
a third polypeptide,
and wherein each of said first to third polypeptides is individually selected from the group consisting of R7, R13, R6 and RC,
wherein
R7 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12,
R13 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14,
R6 is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:13, and
RC is a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:15;
provided that when said first polypeptide is R7, then said second polypeptide is R13 and said third polypeptide is selected from the group consisting of R6 and RC,
provided that when said first polypeptide is R6, then said second polypeptide is R7 and said third polypeptide is selected from the group consisting of R13 and RC,
provided that when said first polypeptide is R13, then said second polypeptide is R7 and said third polypeptide is selected from the group consisting of R6 and RC,
and provided that when said first polypeptide is RC, then said second polypeptide is R7 and said third polypeptide is selected from the group consisting of R13 and R6.

17. The immunogenic composition of any one of claims 1 to 16, which comprises
(i) a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:12, and
(ii) a protein comprising or consisting of an amino acid sequence having at least 70%, preferably at least 80%, more preferably at least 90%, still more preferably at least 95% or in particular 100% sequence identity with a sequence selected from the group consisting of SEQ ID NO:14, SEQ ID NO:13 and SEQ ID NO:15.

18. The immunogenic composition of any one of claims 1 to 17 for use as a medicament, preferably for use as a vaccine.

19. The immunogenic composition of any one of claims 1 to 17 for use in a method of reducing or preventing one or more clinical signs, mortality or fecal shedding caused by a rotavirus infection in a subject or for use in a method of treating or preventing an infection with rotavirus in a subject,
and/or for use in a method for inducing an immune response against rotavirus in a subject.

20. A method of reducing or preventing one or more clinical signs, mortality or fecal shedding caused by a rotavirus infection in a piglet, wherein said method comprises
administering the immunogenic composition of any one of claims 1 to 17 to a sow, and
allowing said piglet to be suckled by said sow.

21. The immunogenic composition according to claim 19, or the method of claim 20, wherein said one or more clinical signs are selected from the group consisting of
diarrhea,
rotavirus colonization, in particular rotavirus colonization of the intestine,
lesions, in particular macroscopic lesions,
decreased average daily weight gain, and
gastroenteritis.

22. The immunogenic composition according to claim 19 or 21, or the method of claim 20 or 21, wherein
said rotavirus infection is an infection with genotype P[23] rotavirus and/or genotype P[7] rotavirus,
said infection with rotavirus is an infection with a genotype P[23] rotavirus and/or genotype P[7] rotavirus, or
said immune response against rotavirus is an immune response against genotype P[23] rotavirus and/or genotype P[7] rotavirus.

23. A method of producing the immunogenic composition of any one of claims 1 to 17, wherein the method comprises the steps of:
(a) permitting infection of susceptible cells in culture with a vector comprising a nucleic acid sequence encoding a polypeptide as specified in any one of claims 1 to 11, wherein said polypeptide is expressed by said vector;
(b) thereafter recovering said polypeptide, in particular in the cell culture supernatant, wherein preferably cell debris is separated from said polypeptide via a separation step, preferably including a micro filtration through at least one filter, preferably two filters, wherein the at least one filter preferably has a pore size of about 1 to about 20 μm and/or about 0.1 μm to about 4 μm;
(c) inactivating the vector by adding binary ethylenimine (BEI) to the mixture of step (b);
(d) neutralizing the BEI by adding sodium thiosulfate to the mixture resulting from step (c); and
(e) concentrating the polypeptide in the mixture resulting from step (d) by removing a portion of the liquid from the mixture by a filtration step utilizing a filter with a filter membrane having a molecular weight cut off of between about 5 kDa and about 100 kDa, preferably between about 10 kDa and about 50 kDa;
and wherein said method comprises the steps of
(f) admixing the mixture remaining after step (e) with at least one immunogenic substance, different from said polypeptide;
(g) and optionally admixing the mixture remaining after step (f) with a further component selected from the group consisting of pharmaceutically acceptable carriers, adjuvants, diluents, excipients, and combinations thereof,
or wherein said method comprises the steps of
(f) admixing the mixture remaining after step (e) with a further component selected from the group consisting of pharmaceutically acceptable carriers, adjuvants, diluents, excipients, and combinations thereof, and
(g) admixing the mixture remaining after step (f) with at least one immunogenic substance, different from said polypeptide.


SEQUENCE LISTING (25 sequences)

SEQ ID NO: 1 - protein, 247 aa, Rotavirus sp.
MASLIYRQLLTNSYTVNLSDEIQEIGSAKSQDVTINPGPFAQTGYAPVNWGAGETNDSTTVEPLLDGPYQPTTFNPPTSYWVLLAPTVEGVIIQGTNNTDRWLATILIEPNVQTTNRIYNLFGQQVTLSVENTSQTQWKFIDVSTTTPTGSYTQHGPLFSTPKLYAVMKFSGRIYTYSGTTPNATTGYYSTTNYDTVNMTSFCDFYIIPRNQEEKCTEYINHGLPPIQNTRNVVPVSLSAREIVHTR

SEQ ID NO: 2 - protein, 160 aa, Rotavirus sp.
LDGPYQPTTFNPPTSYWVLLAPTVEGVIIQGTNNTDRWLATILIEPNVQTTNRIYNLFGQQVTLSVENTSQTQWKFIDVSTTTPTGSYTQHGPLFSTPKLYAVMKFSGRIYTYSGTTPNATTGYYSTTNYDTVNMTSFCDFYIIPRNQEEKCTEYINHGL

SEQ ID NO: 3 - protein, 168 aa, Rotavirus sp.
DSTTVEPLLDGPYQPTTFNPPTSYWVLLAPTVEGVIIQGTNNTDRWLATILIEPNVQTTNRIYNLFGQQVTLSVENTSQTQWKFIDVSTTTPTGSYTQHGPLFSTPKLYAVMKFSGRIYTYSGTTPNATTGYYSTTNYDTVNMTSFCDFYIIPRNQEEKCTEYINHGL

SEQ ID NO: 4 - protein, 167 aa, synthetic construct
  source 1..167: note = Rotavirus consensus sequence
DSTTIEPVLDGPYQPTSFKPPNDYWILLNPTNQQIVLEGTNRTDVWVALLLIEPNVTNQSRQYTLFGETKQITVENNTNKWKFFEMFRNSANAEFQHKRTLTSDTKLAGFLKHGGRVWTFHGETPNATTDYSSTSNLSEIETVIHTEFYIIPRSQESKCNEYINTGL

SEQ ID NO: 5 - protein, 170 aa, synthetic construct
  source 1..170: note = Rotavirus consensus sequence
DSTTVEPVLDGPYQPTTFNPPIEYWTLFAPNDKGVVAELTNNTDIWLAIILIEPNVPQELRTYTIFGQQVNLVIENTSQTKWKFADFRRRSQNDTYVLNDTLLSDTKLQAAMKYGARLFTFTGDTPNAAPQEYGYETNNYSAIEIRSFCDFYIIPRMPREVCRNYINHGL

SEQ ID NO: 6 - protein, 181 aa, Rotavirus sp.
ESTFKSSNITGPHNNTVIEWSNLMNSDIWLLYQKPLDITAPIRLLKHGPENHADVAAFELWYGKAGHTVTSIYYSAISNPNNTVTLTSDSLVLFWNEGQTILDTKTVNFNWNMGGILVRPSRGTRVDICMSDMDNTDGTNFNWIQWKHEFPRSSSNANVSMYVEYYLASSDPYHELKELQR

SEQ ID NO: 7 - protein, 229 aa, Sus scrofa
TKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 8 - protein, 233 aa, Cavia porcellus
RTPQPNPCTCPKCPPPENLGGPSVFIFPPKPKDTLMISLTPRVTCVVVDVSQDEPEVQFTWFVDNKPVGNAETKPRVEQYNTTFRVESVLPIQHQDWLRGKEFKCKVYNKALPAPIEKTISKTKGAPRMPDVYTLPPSRDELSKSKVSVTCLIINFFPADIHVEWASNRVPVSEKEYKNTPPIEDADGSYFLYSKLTVDKSAWDQGTVYTCSVMHEALHNHVTQKAISRSPGK

SEQ ID NO: 9 - skipped

SEQ ID NO: 10 - protein, 8 aa, synthetic construct
  source 1..8: note = linker
GGSGGSGG

SEQ ID NO: 11 - protein, 10 aa, synthetic construct
  source 1..10: note = linker
ASGGGGGGGG

SEQ ID NO: 12 - protein, 401 aa, synthetic construct
  source 1..401: note = fusion protein
MDSTTVEPLLDGPYQPTTFNPPTSYWVLLAPTVEGVIIQGTNNTDRWLATILIEPNVQTTNRIYNLFGQQVTLSVENTSQTQWKFIDVSTTTPTGSYTQHGPLFSTPKLYAVMKFSGRIYTYSGTTPNATTGYYSTTNYDTVNMTSFCDFYIIPRNQEEKCTEYINHGLGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 13 - protein, 400 aa, synthetic construct
  source 1..400: note = fusion protein
MDSTTIEPVLDGPYQPTSFKPPNDYWILLNPTNQQIVLEGTNRTDVWVALLLIEPNVTNQSRQYTLFGETKQITVENNTNKWKFFEMFRNSANAEFQHKRTLTSDTKLAGFLKHGGRVWTFHGETPNATTDYSSTSNLSEIETVIHTEFYIIPRSQESKCNEYINTGLGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 14 - protein, 403 aa, synthetic construct
  source 1..403: note = fusion protein
MDSTTVEPVLDGPYQPTTFNPPIEYWTLFAPNDKGVVAELTNNTDIWLAIILIEPNVPQELRTYTIFGQQVNLVIENTSQTKWKFADFRRRSQNDTYVLNDTLLSDTKLQAAMKYGARLFTFTGDTPNAAPQEYGYETNNYSAIEIRSFCDFYIIPRMPREVCRNYINHGLGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 15 - protein, 414 aa, synthetic construct
  source 1..414: note = fusion protein
MESTFKSSNITGPHNNTVIEWSNLMNSDIWLLYQKPLDITAPIRLLKHGPENHADVAAFELWYGKAGHTVTSIYYSAISNPNNTVTLTSDSLVLFWNEGQTILDTKTVNFNWNMGGILVRPSRGTRVDICMSDMDNTDGTNFNWIQWKHEFPRSSSNANVSMYVEYYLASSDPYHELKELQRGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGK

SEQ ID NO: 16 - protein, 579 aa, synthetic construct
  source 1..579: note = fusion protein
MDSTTVEPLLDGPYQPTTFNPPTSYWVLLAPTVEGVIIQGTNNTDRWLATILIEPNVQTTNRIYNLFGQQVTLSVENTSQTQWKFIDVSTTTPTGSYTQHGPLFSTPKLYAVMKFSGRIYTYSGTTPNATTGYYSTTNYDTVNMTSFCDFYIIPRNQEEKCTEYINHGLGGSTKTKPPCPICPACESPGPSVFIFPPKPKDTLMISRTPQVTCVVVDVSQENPEVQFSWYVDGVEVHTAQTRPKEEQFNSTYRVVSVLPIQHQDWLNGKEFKCKVNNKDLPAPITRIISKAKGQTREPQVYTLPPHAEELSRSKVSITCLVIGFYPPDIDVEWQRNGQPEPEGNYRTTPPQQDVDGTYFLYSKFSVDKASWQGGGIFQCAVMHEALHNHYTQKSISKTPGKGGSGGSGGDSTTVEPVLDGPYQPTTFNPPIEYWTLFAPNDKGVVAELTNNTDIWLAIILIEPNVPQELRTYTIFGQQVNLVIENTSQTKWKFADFRRRSQNDTYVLNDTLLSDTKLQAAMKYGARLFTFTGDTPNAAPQEYGYETNNYSAIEIRSFCDFYIIPRMPREVCRNYINHGL

SEQ ID NO: 17 - DNA, 1206 bp, synthetic construct
  source 1..1206: mol_type = other DNA; note = encodes a fusion protein
atggatagtaccacagtagaaccactgttggacggcccctaccaacctactacgtttaaccccccaacctcatactgggtgttgctcgcgcctacagtcgagggtgtcataatccaaggtacgaataacaccgatcgctggttggctactatccttattgaaccgaatgttcagaccacaaatcgcatttacaacttgttcggacagcaggtgaccctttcagttgagaacacgagtcaaacccaatggaaatttatcgacgtttctaccacaacacctaccggcagctatacgcaacacggcccactctttagcacccctaagctgtatgcagtaatgaagtttagcggacgcatttacacatactcaggtacaactcctaatgctactacgggctactatagcactactaattatgatacggtgaatatgacaagtttctgtgatttctacatcatccccagaaaccaggaggagaaatgtactgaatatataaatcatggactgggcggctcgaccaagaccaagcccccctgtcctatttgcccggcatgcgagagtccgggtccgtccgtctttattttccccccaaagccaaaagatacgctgatgatcagccgcaccccccaggtcacatgcgtggttgtagatgtaagccaagaaaatcccgaagtgcagttttcctggtatgtcgatggcgtggaggttcacacagcacagacgcggccgaaggaagagcagtttaattccacgtaccgagtggtgagtgtgctgcctattcagcatcaggattggttgaacggaaaggagttcaagtgcaaggtgaacaacaaagacctgcccgcaccaatcactcgcataatcagcaaggctaagggacagacacgcgagccacaggtgtacaccctgccaccgcatgcagaagagctgagccgatccaaggtatccataacctgcctggtgatcggattctacccccccgatatcgacgtcgagtggcagaggaatggccagccggagccagagggcaactaccggaccacgcctccgcagcaggatgtcgatggtacatattttctgtacagcaaatttagcgtggacaaggccagttggcagggcggcggtatcttccagtgtgctgtcatgcacgaggcactccacaatcattacacccagaaatccatttcgaagacgccgggcaagtag

SEQ ID NO: 18 - DNA, 1203 bp, synthetic construct
  source 1..1203: mol_type = other DNA; note = encodes a fusion protein
atggatagtactactatcgaaccagttttggatgggccataccagccaacatcgtttaaaccaccaaatgactattggattttattgaaccctactaatcaacagattgtgctggaaggcacaaacagaacagatgtttgggtcgcgctcctgctaatagagccgaacgtaaccaaccagtcccgccaatacaccctattcggtgaaacaaagcaaataactgttgaaaataacacgaataagtggaagttttttgaaatgtttagaaactcagcaaatgccgagtttcagcataaacggacgttgacttctgacaccaagctagcgggtttccttaaacatggcggccgcgtctggacgttccacggagagacccccaatgccaccaccgactactctagcacaagcaatctctcagagatagaaaccgtaatacatactgagttctatatcataccccgaagtcaagagtccaagtgcaacgagtatatcaatacggggttgggtggatctaccaaaaccaagcccccctgccctatttgtccagcgtgcgagtcgccgggtcccagtgtatttatttttccaccgaagcctaaagacacacttatgatcagccgcactccccaagtcacttgtgtggtagttgacgtgagccaagaaaatccggaagtccagttttcatggtatgtcgatggggtagaggtgcacacggctcaaacgaggcctaaagaagagcaattcaattcaacatatcgtgtggtctccgtccttcctatacagcaccaagattggttaaacggaaaagaatttaaatgtaaagtgaataacaaggatctgccggctcccattacgcggatcattagcaaagcaaagggacagacgcgtgagcctcaggtttacactctaccgccacatgcagaggaactctcgcgaagtaaggtgtctataacgtgtctggtaattgggttctacccgcctgacatcgacgttgagtggcagagaaacggtcagccggaaccggagggcaactacaggacaaccccccctcagcaagatgtagatggcacgtacttcctctattctaagttctcggttgacaaggcctcatggcagggcggagggatcttccaatgcgctgtgatgcatgaagcgttacacaatcactatacacaaaagtccatatccaaaactccaggaaaatga

SEQ ID NO: 19 - DNA, 1221 bp, synthetic construct
  source 1..1221: mol_type = other DNA; note = encodes fusion protein
atgacttatccgcgccgacgatatcgccgaagaaggcatagaccgcggtctcatctaggacaaatattgcggcgccgcccatggttggtacatcctcgtcatcgataccgctggcggaggaagaacggcatctttaatactaggctctcccgaacgttcgggtatacagtcaaggctactacagtcaccacaccgagttgggcagtggacatgatgcgttttaatatcgacgatttcgtacctcccggtgggggtactaataaaatatccattcccttcgagtactacaggatacgcaaagttaaggtggaattttggccttgttcgccaattacgcaaggcgatagaggggtcggctcgaccgccgtgattctcgacgacaacttcgttacaaaagctactgcgttaacatatgatccatacgtcaactactcttcaagacacacgatacctcaaccattttcctaccacagccgttatttcactcctaagcccgtactagactccacaatagactacttccagcctaataataagagaaaccagttatggcttagacttcaaacctcacggaacgttgaccacgttggcctaggcacggcctttgagaatagtaaatatgatcaggactataacatccgtgtaacgatgtacgtacagtttcgcgagtttaaccttaaagacccccccctcgagcccggaggatcagatagcacgacagttgaaccggtgctggatgggccatatcagcctaccacgttcaatccaccaattgaatactggaccctcttcgcgccgaacgacaaaggtgtggtagctgagttaactaataatactgacatctggttggctatcatcctgattgaaccaaatgttccgcaggaattgcgtacctatactattttcgggcagcaagtaaaccttgtcatagagaatacaagtcaaaccaaatggaagtttgccgattttagacgtcggtctcagaatgatacgtatgtcctgaatgatacgttactatctgatacaaaactgcaagccgcaatgaagtatggagcaaggctatttacttttactggagataccccgaatgcggcgccgcaagaatatggttacgaaacgaataactatagcgcaatagagattaggtcgttttgtgacttctacatcattcccaggatgcctcgggaagtgtgccgaaactacataaaccacggtctttga

SEQ ID NO: 20 - DNA, 1245 bp, synthetic construct
  source 1..1245: mol_type = other DNA; note = encodes fusion protein
atggaatctacattcaaatcatcaaatataactggtccacacaataacacagtcattgaatggagtaatttaatgaattctgatatttggttattgtatcaaaaaccattggatataactgcaccaatcagattattaaaacatggaccggaaaatcatgctgatgtagcagcttttgaattatggtatggtaaagctggtcataccgtgacatcaatatattattcagcaatatctaatcctaataatactgttacgttaacgtcggattcattagttctattttggaacgaaggtcaaacgatactggatacaaagacagtcaattttaattggaatatgggtggtatattagttagaccgtcaagaggtacacgtgtggacatttgtatgtctgatatggacaatacagatggtactaattttaattggattcaatggaagcatgagttcccccgtagtagtagtaatgctaatgttagtatgtatgttgaatattatctagcaagtagtgatccataccatgaactcaaagagttgcaaagaggcggctcgaccaagaccaagcccccctgtcctatttgcccggcatgcgagagtccgggtccgtccgtctttattttccccccaaagccaaaagatacgctgatgatcagccgcaccccccaggtcacatgcgtggttgtagatgtaagccaagaaaatcccgaagtgcagttttcctggtatgtcgatggcgtggaggttcacacagcacagacgcggccgaaggaagagcagtttaattccacgtaccgagtggtgagtgtgctgcctattcagcatcaggattggttgaacggaaaggagttcaagtgcaaggtgaacaacaaagacctgcccgcaccaatcactcgcataatcagcaaggctaagggacagacacgcgagccacaggtgtacaccctgccaccgcatgcagaagagctgagccgatccaaggtatccataacctgcctggtgatcggattctacccccccgatatcgacgtcgagtggcagaggaatggccagccggagccagagggcaactaccggaccacgcctccgcagcaggatgtcgatggtacatattttctgtacagcaaatttagcgtggacaaggccagttggcagggcggcggtatcttccagtgtgctgtcatgcacgaggcactccacaatcattacacccagaaatccatttcgaagacgccgggcaagtag

SEQ ID NO: 21 - DNA, 1740 bp, synthetic construct
  source 1..1740: mol_type = other DNA; note = encodes fusion protein
atggacagcacgacggttgaaccgctactggacggaccataccaacctactacatttaatcctccaacaagttattgggttctgcttgctcctacagtcgagggagtgataatacaggggacaaataatacggaccggtggttagctacaattttgatcgaaccgaatgtgcaaacgactaatcgcatatacaatttattcggccaacaggtaaccttaagtgtggagaacacctcgcaaactcaatggaaatttattgatgtctccacaacaacccccactggatcgtacactcaacacgggccgctcttctctacccctaaactatacgcggttatgaaatttagtgggagaatctacacttattctggtactacacccaatgcgaccactggttactattcaaccacaaactacgacactgtcaatatgacgtccttttgtgatttttatataatccctaggaatcaagaggagaagtgtacggaatacattaaccatggtctggggggcagcacgaaaacaaaaccgccatgccccatctgccctgcctgcgaaagtcccggcccttccgttttcatttttcccccaaagcccaaggacacgctaatgatttccaggacaccacaggtcacgtgtgtggttgtggatgttagccaggagaatccggaagttcagttttcgtggtatgtagatggggtagaagtgcatacagcccagacgcgaccaaaagaagagcagttcaacagcacctatcgtgttgtaagtgtattaccgatacaacaccaagactggcttaatggtaaagagttcaaatgcaaggtaaacaataaggatctaccggcgcctataacgagaatcatttcaaaggctaagggacaaacaagggagccgcaagtgtacaccttgcccccccacgccgaggaattgagtaggtcaaaagtctcgataacttgtttggttatagggttctatcctccagacatcgatgtggaatggcaacggaacgggcaacccgaacctgaaggcaactatcgcactaccccaccgcaacaggatgtcgacggtacttattttttgtactccaagttttctgtagacaaggcatcatggcagggcggaggtatttttcaatgtgctgtcatgcatgaagcactccacaaccattacacccagaaatctatttcaaagacacccggaaaaggcggatcagggggatcaggaggcgatagcactacggtcgagccggttctggacggaccttatcagcctaccacttttaatcccccgatagaatattggaccctctttgcaccaaacgacaagggcgtcgtagcagagctaacgaacaacaccgatatctggttagcaattatcctcatcgagccgaacgtaccacaggaattacggacgtacaccatcttcgggcagcaagtcaacctcgtgattgaaaacacgtcccagacgaagtggaaattcgcggacttccgtagacgttcgcaaaacgatacgtatgtgctgaatgatactcttctatcggacactaagcttcaggccgctatgaaatacggcgcccgactcttcacattcactggtgatacacccaacgccgcgccacaggagtatggttacgagaccaataactatagcgcaatcgagattcgctctttttgtgatttctatataataccacgaatgccccgtgaggtatgccgaaattatatcaatcatggtctttga

SEQ ID NO: 22 - DNA, 25 bp, synthetic construct
  source 1..25: mol_type = other DNA; note = primer sequence
gctagggayaaaattgttgaaggta

SEQ ID NO: 23 - DNA, 23 bp, synthetic construct
  source 1..23: mol_type = other DNA; note = primer sequence
attggcaaatttcctattcctcc

SEQ ID NO: 24 - DNA, 23 bp, synthetic construct
  source 1..23: mol_type = other DNA; note = probe sequence
atgaatggaaatgaytttcaaac

SEQ ID NO: 25 - DNA, 23 bp, synthetic construct
  source 1..23: mol_type = other DNA; note = probe sequence
atgaatggaaataattttcaaac